SEQUENCE LISTING (96 sequences)

SEQ ID NO: 1 - protein, 7 aa, synthetic construct
  REGION 1..7: note = Description of Artificial Sequence: Synthetic peptide
GYNFKTY

SEQ ID NO: 2 - protein, 7 aa, synthetic construct
  REGION 1..7: note = Description of Artificial Sequence: Synthetic peptide
GYTFTTY

SEQ ID NO: 3 - DNA, 21 bp, synthetic construct
  misc_feature 1..21: note = Description of Artificial Sequence: Synthetic oligonucleotide
  source 1..21: mol_type = other DNA
gggtataccttcacaacctat

SEQ ID NO: 4 - protein, 6 aa, synthetic construct
  REGION 1..6: note = Description of Artificial Sequence: Synthetic peptide
NTYSGM

SEQ ID NO: 5 - DNA, 18 bp, synthetic construct
  misc_feature 1..18: note = Description of Artificial Sequence: Synthetic oligonucleotide
  source 1..18: mol_type = other DNA
aacacctactctggaatg

SEQ ID NO: 6 - protein, 7 aa, synthetic construct
  REGION 1..7: note = Description of Artificial Sequence: Synthetic peptide
PLLGLDY

SEQ ID NO: 7 - DNA, 21 bp, synthetic construct
  misc_feature 1..21: note = Description of Artificial Sequence: Synthetic oligonucleotide
  source 1..21: mol_type = other DNA
cctctcctgggacttgactat

SEQ ID NO: 8 - protein, 12 aa, synthetic construct
  REGION 1..12: note = Description of Artificial Sequence: Synthetic peptide
KSSQSLLHSDGK

SEQ ID NO: 9 - DNA, 36 bp, synthetic construct
  misc_feature 1..36: note = Description of Artificial Sequence: Synthetic oligonucleotide
  source 1..36: mol_type = other DNA
aagtcaagtcagagcctcttacatagtgatggaaag

SEQ ID NO: 10 - protein, 7 aa, synthetic construct
  REGION 1..7: note = Description of Artificial Sequence: Synthetic peptide
LVSKLES

SEQ ID NO: 11 - DNA, 21 bp, synthetic construct
  misc_feature 1..21: note = Description of Artificial Sequence: Synthetic oligonucleotide
  source 1..21: mol_type = other DNA
ctggtgtctaaactggaatct

SEQ ID NO: 12 - protein, 9 aa, synthetic construct
  REGION 1..9: note = Description of Artificial Sequence: Synthetic peptide
LQHTHFPLT

SEQ ID NO: 13 - DNA, 27 bp, synthetic construct
  misc_feature 1..27: note = Description of Artificial Sequence: Synthetic oligonucleotide
  source 1..27: mol_type = other DNA
ttgcaacatacacattttccgctcacg

SEQ ID NO: 14 - protein, 116 aa, synthetic construct
  REGION 1..116: note = Description of Artificial Sequence: Synthetic polypeptide
QIQLVQSGPELKKPGETVKISCKASGYTFTTYGMSWVKQAPGKGLKWMGWINTYSGMPTYADDFKGRFAFSLETSATTAYLQINNLKNEDTATYFCARPLLGLDYWGQGTTLTVSS

SEQ ID NO: 15 - DNA, 348 bp, synthetic construct
  misc_feature 1..348: note = Description of Artificial Sequence: Synthetic polynucleotide
  source 1..348: mol_type = other DNA
cagatccagttggtacagtctggacctgagctgaagaagcctggagagacagtcaagatctcctgcaaggcctccgggtataccttcacaacctatggaatgagctgggtgaaacaggctccaggaaagggtttaaagtggatgggctggataaacacctactctggaatgccaacatatgctgatgacttcaagggacggtttgccttctctttggaaacctctgccaccactgcctatttgcagatcaacaacctcaaaaatgaggacacggctacatatttctgtgcaagacctctcctgggacttgactattggggccaaggcaccactctcacagtctcctca

SEQ ID NO: 16 - protein, 112 aa, synthetic construct
  REGION 1..112: note = Description of Artificial Sequence: Synthetic polypeptide
DIVMTQTPLSLSVTIGQPASISCKSSQSLLHSDGKTFLNWFLQRPGQSPKLLIYLVSKLESGIPDRFSGSGSGTDFTLKISRVEVEDLGVYYCLQHTHFPLTFGAGTKLEIK

SEQ ID NO: 17 - DNA, 336 bp, synthetic construct
  misc_feature 1..336: note = Description of Artificial Sequence: Synthetic polynucleotide
  source 1..336: mol_type = other DNA
gatattgtgatgacccagactccactgtctttgtcggttaccattggacaaccagcctccatctcttgcaagtcaagtcagagcctcttacatagtgatggaaagacatttttgaattggtttttacagaggccaggccagtctccaaagctcctaatctatctggtgtctaaactggaatctggcatccctgacaggttcagtggcagtggatcagggacagatttcacactgaaaatcagcagagtggaggttgaggatttgggagtttattactgcttgcaacatacacattttccgctcacgttcggtgctgggaccaaactggaaataaaa

SEQ ID NO: 18 - protein, 446 aa, synthetic construct
  REGION 1..446: note = Description of Artificial Sequence: Synthetic polypeptide
EIQLVESGGGLVQPGGSVRLSCAASGYNFKTYGMSWVRQAPGKGLEWMGWINTYSGMPTYADDFKGRFTFSLDTSKNTAYLQINSLRAEDTAVYFCARPLLGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1407 bp, synthetic construct
  misc_feature 1..1407: note = Description of Artificial Sequence: Synthetic polynucleotide
  source 1..1407: mol_type = other DNA
gccgccaccatgggctggtccctgattctgctgttcctggtggctgtggctaccagggtgctgagtgagatccagctggtggagagcggaggaggactggtgcagccaggaggatctgtgaggctgagctgcgcagcatccggatacaacttcaagacctatggcatgtcctgggtgcgccaggcaccaggcaagggactggagtggatgggctggatcaatacctactctggcatgcctacatatgccgacgatttcaagggccggttcaccttttctctggacaccagcaagaacacagcctacctgcagatcaattccctgcgggccgaggacacagccgtgtacttttgtgccagacccctgctgggcctggattattggggccagggcaccctggtgacagtgagctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 219 aa, synthetic construct
  REGION 1..219: note = Description of Artificial Sequence: Synthetic polypeptide
DIQMTQSPSSLSASVGDRVTITCKSSQSLLHSDGKTFLNWFQQKPGKSPKLLIYLVSKLESGVPSRFSGSGSGTDFTLTISSLQPEDFATYYCLQHTHFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 726 bp, synthetic construct
  misc_feature 1..726: note = Description of Artificial Sequence: Synthetic polynucleotide
  source 1..726: mol_type = other DNA
gccgccaccatgggctggtcctgtatcatcctgttcctggtggctacagccacaggagtgcatagtgacatccagatgacacagtcccctagctccctgagcgcctccgtgggcgatagggtgaccatcacatgcaagtctagccagtctctgctgcacagcgacggcaagaccttcctgaactggtttcagcagaagcccggcaagtctcctaagctgctgatctacctggtgtccaagctggagtctggcgtgcccagcagattctctggcagcggctccggcacagactttaccctgacaatctcctctctgcagccagaggatttcgccacctactattgtctgcagcacacccacttccccctgacatttggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 22 - protein, 446 aa, synthetic construct
  REGION 1..446: note = Description of Artificial Sequence: Synthetic polypeptide
QIQLVQSGPELKKPGETVKISCKASGYTFTTYGMSWVKQAPGKGLKWMGWINTYSGMPTYADDFKGRFAFSLETSATTAYLQINNLKNEDTATYFCARPLLGLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1407 bp, synthetic construct
  misc_feature 1..1407: note = Description of Artificial Sequence: Synthetic polynucleotide
  source 1..1407: mol_type = other DNA
gccgccaccatgggctggtccctgattctgctgttcctggtggctgtggctaccagggtgctgagtcagatccagctggtgcagtctggccccgagctgaagaagcctggcgagaccgtgaagatctcttgcaaggccagcggctacacattcaccacatatggcatgagctgggtgaagcaggcaccaggcaagggcctgaagtggatgggctggatcaacacctactccggcatgcccacatatgccgacgatttcaagggccggttcgccttttccctggagacctctgccaccacagcctacctgcagatcaacaatctgaagaatgaggacaccgccacatacttttgtgccagacccctgctgggcctggattattggggccagggcaccacactgacagtgagctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 24 - protein, 219 aa, synthetic construct
  REGION 1..219: note = Description of Artificial Sequence: Synthetic polypeptide
DIVMTQTPLSLSVTIGQPASISCKSSQSLLHSDGKTFLNWFLQRPGQSPKLLIYLVSKLESGIPDRFSGSGSGTDFTLKISRVEVEDLGVYYCLQHTHFPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 726 bp, synthetic construct
  misc_feature 1..726: note = Description of Artificial Sequence: Synthetic polynucleotide
  source 1..726: mol_type = other DNA
gccgccaccatgggctggtcctgtatcatcctgttcctggtggctacagccacaggagtgcatagtgacatcgtgatgacccagacaccactgtctctgagcgtgacaatcggccagcccgcctccatctcttgcaagagctcccagagcctgctgcactccgatggcaagaccttcctgaactggtttctgcagaggccaggacagtcccctaagctgctgatctacctggtgtctaagctggagagcggaatccctgaccggttcagcggatccggatctggaaccgacttcaccctgaagatctctagagtggaggtggaggacctgggcgtgtactattgtctgcagcacacccacttccccctgacatttggcgccggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 26 - protein, 5 aa, synthetic construct
  REGION 1..5: note = Description of Artificial Sequence: Synthetic peptide
TYGMS

SEQ ID NO: 27 - protein, 17 aa, synthetic construct
  REGION 1..17: note = Description of Artificial Sequence: Synthetic peptide
WINTYSGMPTYADDFKG

SEQ ID NO: 28 - protein, 7 aa, synthetic construct
  REGION 1..7: note = Description of Artificial Sequence: Synthetic peptide
PLLGLDY

SEQ ID NO: 29 - protein, 16 aa, synthetic construct
  REGION 1..16: note = Description of Artificial Sequence: Synthetic peptide
KSSQSLLHSDGKTFLN

SEQ ID NO: 30 - DNA, 1380 bp, synthetic construct
  misc_feature 1..1380: note = Description of Artificial Sequence: Synthetic polynucleotide
  source 1..1380: mol_type = other DNA
caggtgcagctgcaggagtccgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagccgaggacacggctgtgtattactgtgcgagagatctcggcgccgttatagcagtggctggtacttccaccccgtacaactggttcgacccctggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 31 - protein, 459 aa, synthetic construct
  REGION 1..459: note = Description of Artificial Sequence: Synthetic polypeptide
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCARDLGAVIAVAGTSTPYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 69 bp, synthetic construct
  misc_feature 1..69: note = Description of Artificial Sequence: Synthetic oligonucleotide
  source 1..69: mol_type = other DNA
atggagacagacacactcctgctatgggtactgctgctctgggttccaggttccactggcgccggatca

SEQ ID NO: 33 - DNA, 1449 bp, synthetic construct
  misc_feature 1..1449: note = Description of Artificial Sequence: Synthetic polynucleotide
  source 1..1449: mol_type = other DNA
atggagacagacacactcctgctatgggtactgctgctctgggttccaggttccactggcgccggatcacaggtgcagctgcaggagtccgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagccgaggacacggctgtgtattactgtgcgagagatctcggcgccgttatagcagtggctggtacttccaccccgtacaactggttcgacccctggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - protein, 23 aa, synthetic construct
  REGION 1..23: note = Description of Artificial Sequence: Synthetic peptide
METDTLLLWVLLLWVPGSTGAGS

SEQ ID NO: 35 - protein, 482 aa, synthetic construct
  REGION 1..482: note = Description of Artificial Sequence: Synthetic polypeptide
METDTLLLWVLLLWVPGSTGAGSQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCARDLGAVIAVAGTSTPYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 660 bp, synthetic construct
  misc_feature 1..660: note = Description of Artificial Sequence: Synthetic polynucleotide
  source 1..660: mol_type = other DNA
gatgttgtgatgactcagtctccactctccctgcccgtcacccctggagagccggcctccatctcctgcaggtctcgtcagagcctcctgcatagcaatggacacaactatttgggttggtacctgcagaagccagggcagtctccacagctcctgatctatctggcttctattcgggcctccgggatccctgacaggttcagtggcagtggatcaggcacagattttacactgaaaatcagcagagtggaggctgaggatgttggcgtttattactgcatgcaagctctacaaacaccctacacttttggccaggggaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 37 - protein, 219 aa, synthetic construct
  REGION 1..219: note = Description of Artificial Sequence: Synthetic polypeptide
DVVMTQSPLSLPVTPGEPASISCRSRQSLLHSNGHNYLGWYLQKPGQSPQLLIYLASIRASGIPDRFSGSGSGTDFTLKISRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 729 bp, synthetic construct
  misc_feature 1..729: note = Description of Artificial Sequence: Synthetic polynucleotide
  source 1..729: mol_type = other DNA
atggagacagacacactcctgctatgggtactgctgctctgggttccaggttccactggcgccggatcagatgttgtgatgactcagtctccactctccctgcccgtcacccctggagagccggcctccatctcctgcaggtctcgtcagagcctcctgcatagcaatggacacaactatttgggttggtacctgcagaagccagggcagtctccacagctcctgatctatctggcttctattcgggcctccgggatccctgacaggttcagtggcagtggatcaggcacagattttacactgaaaatcagcagagtggaggctgaggatgttggcgtttattactgcatgcaagctctacaaacaccctacacttttggccaggggaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 39 - protein, 242 aa, synthetic construct
  REGION 1..242: note = Description of Artificial Sequence: Synthetic polypeptide
METDTLLLWVLLLWVPGSTGAGSDVVMTQSPLSLPVTPGEPASISCRSRQSLLHSNGHNYLGWYLQKPGQSPQLLIYLASIRASGIPDRFSGSGSGTDFTLKISRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 1389 bp, synthetic construct
  misc_feature 1..1389: note = Description of Artificial Sequence: Synthetic polynucleotide
  source 1..1389: mol_type = other DNA
cagatgcagctggtgcagtcggggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtattactgtgcgagagaaagctcgatgactacagtaactacgtatctcctacgggaagtaggggtagggttggactttgactactggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 41 - protein, 462 aa, synthetic construct
  REGION 1..462: note = Description of Artificial Sequence: Synthetic polypeptide
QMQLVQSGGGVVRPGGSLRLSCAASGFTFDDYGMSWVRQAPGKGLEWVSGINWNGGSTGYADSVKGRFTISRDNAKNSLYLQMNSLRAEDTALYYCARESSMTTVTTYLLREVGVGLDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 1458 bp, synthetic construct
  misc_feature 1..1458: note = Description of Artificial Sequence: Synthetic polynucleotide
  source 1..1458: mol_type = other DNA
atggagacagacacactcctgctatgggtactgctgctctgggttccaggttccactggcgccggatcacagatgcagctggtgcagtcggggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtattactgtgcgagagaaagctcgatgactacagtaactacgtatctcctacgggaagtaggggtagggttggactttgactactggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 43 - protein, 485 aa, synthetic construct
  REGION 1..485: note = Description of Artificial Sequence: Synthetic polypeptide
METDTLLLWVLLLWVPGSTGAGSQMQLVQSGGGVVRPGGSLRLSCAASGFTFDDYGMSWVRQAPGKGLEWVSGINWNGGSTGYADSVKGRFTISRDNAKNSLYLQMNSLRAEDTALYYCARESSMTTVTTYLLREVGVGLDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 645 bp, synthetic construct
  misc_feature 1..645: note = Description of Artificial Sequence: Synthetic polynucleotide
  source 1..645: mol_type = other DNA
gaaacgacactcacgcagtctccaggcaccctgtccttgtctccaggggaaagagccaccctctcctgcagggccagtcagactgttagcggcagctacttagcctggtaccagcagaaacctggccagcctcccaggctcctcatctatggtgcatccagcagggccactggcatcccagacaggttcagtggcagtgggtccgggacagacttcactctcaccatcagcagactggagcctgaagattttgcagtgtattactgtcaggactatggtagctcacggacgttcggccaagggaccaaggtggaactcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgttag

SEQ ID NO: 45 - protein, 214 aa, synthetic construct
  REGION 1..214: note = Description of Artificial Sequence: Synthetic polypeptide
ETTLTQSPGTLSLSPGERATLSCRASQTVSGSYLAWYQQKPGQPPRLLIYGASSRATGIPDRFSGSGSGTDFTLTISRLEPEDFAVYYCQDYGSSRTFGQGTKVELKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 46 - DNA, 714 bp, synthetic construct
  misc_feature 1..714: note = Description of Artificial Sequence: Synthetic polynucleotide
  source 1..714: mol_type = other DNA
atggagacagacacactcctgctatgggtactgctgctctgggttccaggttccactggcgccggatcagaaacgacactcacgcagtctccaggcaccctgtccttgtctccaggggaaagagccaccctctcctgcagggccagtcagactgttagcggcagctacttagcctggtaccagcagaaacctggccagcctcccaggctcctcatctatggtgcatccagcagggccactggcatcccagacaggttcagtggcagtgggtccgggacagacttcactctcaccatcagcagactggagcctgaagattttgcagtgtattactgtcaggactatggtagctcacggacgttcggccaagggaccaaggtggaactcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgttag

SEQ ID NO: 47 - protein, 237 aa, synthetic construct
  REGION 1..237: note = Description of Artificial Sequence: Synthetic polypeptide
METDTLLLWVLLLWVPGSTGAGSETTLTQSPGTLSLSPGERATLSCRASQTVSGSYLAWYQQKPGQPPRLLIYGASSRATGIPDRFSGSGSGTDFTLTISRLEPEDFAVYYCQDYGSSRTFGQGTKVELKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 48 - DNA, 1374 bp, synthetic construct
  misc_feature 1..1374: note = Description of Artificial Sequence: Synthetic polynucleotide
  source 1..1374: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagacgatattgtagtggtggtagctgctacttagttggaacggggtctgaattggactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 49 - protein, 457 aa, synthetic construct
  REGION 1..457: note = Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARRYCSGGSCYLVGTGSELDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 1443 bp, synthetic construct
  misc_feature 1..1443: note = Description of Artificial Sequence: Synthetic polynucleotide
  source 1..1443: mol_type = other DNA
atggagacagacacactcctgctatgggtactgctgctctgggttccaggttccactggcgccggatcacaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagacgatattgtagtggtggtagctgctacttagttggaacggggtctgaattggactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 51 - protein, 480 aa, synthetic construct
  REGION 1..480: note = Description of Artificial Sequence: Synthetic polypeptide
METDTLLLWVLLLWVPGSTGAGSQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARRYCSGGSCYLVGTGSELDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - DNA, 642 bp, synthetic construct
  misc_feature 1..642: note = Description of Artificial Sequence: Synthetic polynucleotide
  source 1..642: mol_type = other DNA
gacatccagatgacccagtctccttccaccctgtctgcatctgtaggagacagagtcaccataacttgccgggccagtcagagcattagtagctggttggcctggtatcagcagaaaccagggaaagcccctaaactcctaatctataaggcgtctagtttagaaattggggtcccatcaaggttcagcggcagtggatatgggacagaattcactctcaccatcagcagcctgcagcctgatgatttcgcaacttattattgcctacagtatagtactcattcgacgttcggccaagggaccagggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgttag

SEQ ID NO: 53 - protein, 213 aa, synthetic construct
  REGION 1..213: note = Description of Artificial Sequence: Synthetic polypeptide
DIQMTQSPSTLSASVGDRVTITCRASQSISSWLAWYQQKPGKAPKLLIYKASSLEIGVPSRFSGSGYGTEFTLTISSLQPDDFATYYCLQYSTHSTFGQGTRVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 54 - DNA, 711 bp, synthetic construct
  misc_feature 1..711: note = Description of Artificial Sequence: Synthetic polynucleotide
  source 1..711: mol_type = other DNA
atggagacagacacactcctgctatgggtactgctgctctgggttccaggttccactggcgccggatcagacatccagatgacccagtctccttccaccctgtctgcatctgtaggagacagagtcaccataacttgccgggccagtcagagcattagtagctggttggcctggtatcagcagaaaccagggaaagcccctaaactcctaatctataaggcgtctagtttagaaattggggtcccatcaaggttcagcggcagtggatatgggacagaattcactctcaccatcagcagcctgcagcctgatgatttcgcaacttattattgcctacagtatagtactcattcgacgttcggccaagggaccagggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgttag

SEQ ID NO: 55 - protein, 236 aa, synthetic construct
  REGION 1..236: note = Description of Artificial Sequence: Synthetic polypeptide
METDTLLLWVLLLWVPGSTGAGSDIQMTQSPSTLSASVGDRVTITCRASQSISSWLAWYQQKPGKAPKLLIYKASSLEIGVPSRFSGSGYGTEFTLTISSLQPDDFATYYCLQYSTHSTFGQGTRVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 56 - DNA, 383 bp, synthetic construct
  misc_feature 1..383: note = Description of Artificial Sequence: Synthetic polynucleotide
  source 1..383: mol_type = other DNA
caggtgcagctgcaggagtccgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagccgaggacacggctgtgtattactgtgcgagagatctcggcgccgttatagcagtggctggtacttccaccccgtacaactggttcgacccctggggccagggaaccctggtcaccgtctc

SEQ ID NO: 57 - protein, 128 aa, synthetic construct
  REGION 1..128: note = Description of Artificial Sequence: Synthetic polypeptide
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCARDLGAVIAVAGTSTPYNWFDPWGQGTLVTVS

SEQ ID NO: 58 - DNA, 335 bp, synthetic construct
  misc_feature 1..335: note = Description of Artificial Sequence: Synthetic polynucleotide
  source 1..335: mol_type = other DNA
gatgttgtgatgactcagtctccactctccctgcccgtcacccctggagagccggcctccatctcctgcaggtctcgtcagagcctcctgcatagcaatggacacaactatttgggttggtacctgcagaagccagggcagtctccacagctcctgatctatctggcttctattcgggcctccgggatccctgacaggttcagtggcagtggatcaggcacagattttacactgaaaatcagcagagtggaggctgaggatgttggcgtttattactgcatgcaagctctacaaacaccctacacttttggccaggggaccaagctggagatcaa

SEQ ID NO: 59 - protein, 112 aa, synthetic construct
  REGION 1..112: note = Description of Artificial Sequence: Synthetic polypeptide
DVVMTQSPLSLPVTPGEPASISCRSRQSLLHSNGHNYLGWYLQKPGQSPQLLIYLASIRASGIPDRFSGSGSGTDFTLKISRVEAEDVGVYYCMQALQTPYTFGQGTKLEIK

SEQ ID NO: 60 - protein, 8 aa, synthetic construct
  REGION 1..8: note = Description of Artificial Sequence: Synthetic peptide
GFTFSSYG

SEQ ID NO: 61 - protein, 8 aa, synthetic construct
  REGION 1..8: note = Description of Artificial Sequence: Synthetic peptide
ISYDGSNK

SEQ ID NO: 62 - protein, 22 aa, synthetic construct
  REGION 1..22: note = Description of Artificial Sequence: Synthetic peptide
ARDLGAVIAVAGTSTPYNWFDP

SEQ ID NO: 63 - protein, 11 aa, synthetic construct
  REGION 1..11: note = Description of Artificial Sequence: Synthetic peptide
QSLLHSNGHNY

SEQ ID NO: 64 - skipped

SEQ ID NO: 65 - protein, 9 aa, synthetic construct
  REGION 1..9: note = Description of Artificial Sequence: Synthetic peptide
MQALQTPYT

SEQ ID NO: 66 - DNA, 392 bp, synthetic construct
  misc_feature 1..392: note = Description of Artificial Sequence: Synthetic polynucleotide
  source 1..392: mol_type = other DNA
cagatgcagctggtgcagtcggggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtattactgtgcgagagaaagctcgatgactacagtaactacgtatctcctacgggaagtaggggtagggttggactttgactactggggccagggcaccctggtcaccgtctc

SEQ ID NO: 67 - protein, 131 aa, synthetic construct
  REGION 1..131: note = Description of Artificial Sequence: Synthetic polypeptide
QMQLVQSGGGVVRPGGSLRLSCAASGFTFDDYGMSWVRQAPGKGLEWVSGINWNGGSTGYADSVKGRFTISRDNAKNSLYLQMNSLRAEDTALYYCARESSMTTVTTYLLREVGVGLDFDYWGQGTLVTVS

SEQ ID NO: 68 - DNA, 322 bp, synthetic construct
  misc_feature 1..322: note = Description of Artificial Sequence: Synthetic polynucleotide
  source 1..322: mol_type = other DNA
gaaacgacactcacgcagtctccaggcaccctgtccttgtctccaggggaaagagccaccctctcctgcagggccagtcagactgttagcggcagctacttagcctggtaccagcagaaacctggccagcctcccaggctcctcatctatggtgcatccagcagggccactggcatcccagacaggttcagtggcagtgggtccgggacagacttcactctcaccatcagcagactggagcctgaagattttgcagtgtattactgtcaggactatggtagctcacggacgttcggccaagggaccaaggtggaactcaaac

SEQ ID NO: 69 - protein, 107 aa, synthetic construct
  REGION 1..107: note = Description of Artificial Sequence: Synthetic polypeptide
ETTLTQSPGTLSLSPGERATLSCRASQTVSGSYLAWYQQKPGQPPRLLIYGASSRATGIPDRFSGSGSGTDFTLTISRLEPEDFAVYYCQDYGSSRTFGQGTKVELK

SEQ ID NO: 70 - protein, 8 aa, synthetic construct
  REGION 1..8: note = Description of Artificial Sequence: Synthetic peptide
GFTFDDYG

SEQ ID NO: 71 - protein, 8 aa, synthetic construct
  REGION 1..8: note = Description of Artificial Sequence: Synthetic peptide
INWNGGST

SEQ ID NO: 72 - protein, 25 aa, synthetic construct
  REGION 1..25: note = Description of Artificial Sequence: Synthetic peptide
ARESSMTTVTTYLLREVGVGLDFDY

SEQ ID NO: 73 - protein, 7 aa, synthetic construct
  REGION 1..7: note = Description of Artificial Sequence: Synthetic peptide
QTVSGSY

SEQ ID NO: 74 - skipped

SEQ ID NO: 75 - protein, 8 aa, synthetic construct
  REGION 1..8: note = Description of Artificial Sequence: Synthetic peptide
QDYGSSRT

SEQ ID NO: 76 - DNA, 377 bp, synthetic construct
  misc_feature 1..377: note = Description of Artificial Sequence: Synthetic polynucleotide
  source 1..377: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagacgatattgtagtggtggtagctgctacttagttggaacggggtctgaattggactactggggccagggaaccctggtcaccgtctc

SEQ ID NO: 77 - protein, 126 aa, synthetic construct
  REGION 1..126: note = Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARRYCSGGSCYLVGTGSELDYWGQGTLVTVS

SEQ ID NO: 78 - DNA, 319 bp, synthetic construct
  misc_feature 1..319: note = Description of Artificial Sequence: Synthetic polynucleotide
  source 1..319: mol_type = other DNA
gacatccagatgacccagtctccttccaccctgtctgcatctgtaggagacagagtcaccataacttgccgggccagtcagagcattagtagctggttggcctggtatcagcagaaaccagggaaagcccctaaactcctaatctataaggcgtctagtttagaaattggggtcccatcaaggttcagcggcagtggatatgggacagaattcactctcaccatcagcagcctgcagcctgatgatttcgcaacttattattgcctacagtatagtactcattcgacgttcggccaagggaccagggtggaaatcaaac

SEQ ID NO: 79 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLEIGVPSRFSGSGYGTEFTLTISSLQPDDFATYYCLQYSTHSTFGQGTRVEIK

SEQ ID NO: 80 - protein, 8 aa, synthetic construct
  REGION 1..8: note = Description of Artificial Sequence: Synthetic peptide
GGSFSGYY

SEQ ID NO: 81 - protein, 7 aa, synthetic construct
  REGION 1..7: note = Description of Artificial Sequence: Synthetic peptide
INHSGST

SEQ ID NO: 82 - protein, 21 aa, synthetic construct
  REGION 1..21: note = Description of Artificial Sequence: Synthetic peptide
ARRYCSGGSCYLVGTGSELDY

SEQ ID NO: 83 - protein, 6 aa, synthetic construct
  REGION 1..6: note = Description of Artificial Sequence: Synthetic peptide
QSISSW

SEQ ID NO: 84 - skipped

SEQ ID NO: 85 - protein, 8 aa, synthetic construct
  REGION 1..8: note = Description of Artificial Sequence: Synthetic peptide
LQYSTHST

SEQ ID NO: 86 - protein, 20 aa, Mus sp.
SEEEEIEKREEIHHQLQMLL

SEQ ID NO: 87 - DNA, 20 bp, synthetic construct
  misc_feature 1..20: note = Description of Artificial Sequence: Synthetic primer
  source 1..20: mol_type = other DNA
ctacaggggacaccacccac

SEQ ID NO: 88 - DNA, 22 bp, synthetic construct
  misc_feature 1..22: note = Description of Artificial Sequence: Synthetic primer
  source 1..22: mol_type = other DNA
tacagtactgacccgtccactc

SEQ ID NO: 89 - DNA, 24 bp, synthetic construct
  misc_feature 1..24: note = Description of Artificial Sequence: Synthetic primer
  source 1..24: mol_type = other DNA
ctctggctcctagcaccatgaaga

SEQ ID NO: 90 - DNA, 26 bp, synthetic construct
  misc_feature 1..26: note = Description of Artificial Sequence: Synthetic primer
  source 1..26: mol_type = other DNA
gtaaaacgcagctcagtaacagtccg

SEQ ID NO: 91 - DNA, 6414 bp, synthetic construct
  misc_feature 1..6414: note = Description of Artificial Sequence: Synthetic polynucleotide
  source 1..6414: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgaccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagaactcgttgctgccgcgcgcgccaccagacataatagctgacagactaacagactgttcctttccatgggtcttttctgcagtcaccgtcctgacacgaagcttgccgccaccatgggctggtccctgattctgctgttcctggtggctgtggctaccagggtgctgagtgagatccagctggtggagagcggaggaggactggtgcagccaggaggatctgtgaggctgagctgcgcagcatccggatacaacttcaagacctatggcatgtcctgggtgcgccaggcaccaggcaagggactggagtggatgggctggatcaatacctactctggcatgcctacatatgccgacgatttcaagggccggttcaccttttctctggacaccagcaagaacacagcctacctgcagatcaattccctgcgggccgaggacacagccgtgtacttttgtgccagacccctgctgggcctggattattggggccagggcaccctggtgacagtgagctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttcgctcgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcagccggccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtattcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 92 - DNA, 5733 bp, synthetic construct
  misc_feature 1..5733: note = Description of Artificial Sequence: Synthetic polynucleotide
  source 1..5733: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgaccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagaactcgttgctgccgcgcgcgccaccagacataatagctgacagactaacagactgttcctttccatgggtcttttctgcagtcaccgtcctgacacgaagcttgccgccaccatgggctggtcctgtatcatcctgttcctggtggctacagccacaggagtgcatagtgacatccagatgacacagtcccctagctccctgagcgcctccgtgggcgatagggtgaccatcacatgcaagtctagccagtctctgctgcacagcgacggcaagaccttcctgaactggtttcagcagaagcccggcaagtctcctaagctgctgatctacctggtgtccaagctggagtctggcgtgcccagcagattctctggcagcggctccggcacagactttaccctgacaatctcctctctgcagccagaggatttcgccacctactattgtctgcagcacacccacttccccctgacatttggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttcgctagcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcagctagccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtattcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 93 - DNA, 6414 bp, synthetic construct
  misc_feature 1..6414: note = Description of Artificial Sequence: Synthetic polynucleotide
  source 1..6414: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgaccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagaactcgttgctgccgcgcgcgccaccagacataatagctgacagactaacagactgttcctttccatgggtcttttctgcagtcaccgtcctgacacgaagcttgccgccaccatgggctggtccctgattctgctgttcctggtggctgtggctaccagggtgctgagtcagatccagctggtgcagtctggccccgagctgaagaagcctggcgagaccgtgaagatctcttgcaaggccagcggctacacattcaccacatatggcatgagctgggtgaagcaggcaccaggcaagggcctgaagtggatgggctggatcaacacctactccggcatgcccacatatgccgacgatttcaagggccggttcgccttttccctggagacctctgccaccacagcctacctgcagatcaacaatctgaagaatgaggacaccgccacatacttttgtgccagacccctgctgggcctggattattggggccagggcaccacactgacagtgagctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttcgctcgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcagccggccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtattcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 94 - DNA, 5733 bp, synthetic construct
  misc_feature 1..5733: note = Description of Artificial Sequence: Synthetic polynucleotide
  source 1..5733: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgaccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagaactcgttgctgccgcgcgcgccaccagacataatagctgacagactaacagactgttcctttccatgggtcttttctgcagtcaccgtcctgacacgaagcttgccgccaccatgggctggtcctgtatcatcctgttcctggtggctacagccacaggagtgcatagtgacatcgtgatgacccagacaccactgtctctgagcgtgacaatcggccagcccgcctccatctcttgcaagagctcccagagcctgctgcactccgatggcaagaccttcctgaactggtttctgcagaggccaggacagtcccctaagctgctgatctacctggtgtctaagctggagagcggaatccctgaccggttcagcggatccggatctggaaccgacttcaccctgaagatctctagagtggaggtggaggacctgggcgtgtactattgtctgcagcacacccacttccccctgacatttggcgccggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttcgctagcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcagctagccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtattcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 95 - protein, 116 aa, synthetic construct
  REGION 1..116: note = Description of Artificial Sequence: Synthetic polypeptide
EIQLVESGGGLVQPGGSVRLSCAASGYNFKTYGMSWVRQAPGKGLEWMGWINTYSGMPTYADDFKGRFTFSLDTSKNTAYLQINSLRAEDTAVYFCARPLLGLDYWGQGTLVTVSS

SEQ ID NO: 96 - protein, 112 aa, synthetic construct
  REGION 1..112: note = Description of Artificial Sequence: Synthetic polypeptide
DIQMTQSPSSLSASVGDRVTITCKSSQSLLHSDGKTFLNWFQQKPGKSPKLLIYLVSKLESGVPSRFSGSGSGTDFTLTISSLQPEDFATYYCLQHTHFPLTFGQGTKVEIK

(19) United States
(12) Patent Application Publication
(10) Pub. No.: US 2026/0116996 A1
(43) Pub. Date: Apr. 30, 2026

(54) COMPOSITIONS AND METHODS FOR TREATING SERPIN B13 DISORDERS

(71) Applicant: REGENTS OF THE UNIVERSITY OF MINNESOTA, Mineapolis, MN (US)

(72) Inventor: Jan CZYZYK, Minneapolis, MN (US)

(73) Assignee: REGENTS OF THE UNIVERSITY OF MINNESOTA, Mineapolis, MN (US)

(21) Appl. No.: 19/284,332

(22) Filed: Jul. 29, 2025

Related U.S. Application Data
(63) Continuation of application No. 17/633,012, now Pat. No. 12,441,811.
(60) Provisional application No. 62/883,443, filed on Aug. 6, 2019.
(60) Provisional application No. 63/040,356, filed on Jun. 17, 2020.

(51) Int. Cl. C07K 16/38

(52) U.S. Cl. CPC C07K 16/38; C07K 2317/21; C07K 2317/24; C07K 2317/565; C07K 2317/76

(57) ABSTRACT

Provided herein are anti-OVA-serine proteinase inhibitor (serpin) B13 monoclonal antibodies and antigen-binding antibody fragments that selectively and specifically bind to an epitope of serpin B13, compositions containing these antibodies and antibody fragments, and methods of using these antibodies and antibody fragments. These antibodies and antigen-binding fragments thereof are useful for inhibiting serpin B13 and for treating serpin B13-related diseases, e.g., type I diabetes.

### CROSS-REFERENCE TO RELATED APPLICATIONS

This application is a continuation of U.S. patent application Ser. No. 17/633,012, filed Feb. 4, 2022, which is a 35 U.S.C. § 371 application of International Application Serial No. PCT/US2020/044729, filed Aug. 3, 2020; which claims the benefit of U.S. Provisional Application Ser. No. 62/883,443 that was filed on Aug. 6, 2019 and U.S. Provisional Application Ser. No. 63/040,356 that was filed on Jun. 17, 2020. The entire content of the applications referenced above are hereby incorporated by reference herein.

### SEQUENCE LISTING

The instant application contains a Sequence Listing which has been submitted electronically in XML format and is hereby incorporated by reference in its entirety. Said XML copy, created on Dec. 17, 2025, is named 09531_469US2_SL.xml and is 171,337 bytes in size.

### BACKGROUND

Intracellular (clade B) OVA-serpin protease inhibitors play an important role in tissue homeostasis by protecting cells from death in response to hypo-osmotic stress, heat shock, and other stimuli. High levels of anti-serpinB13 Abs were accompanied by low levels of anti-insulin autoantibodies, reduced numbers of islet-associated T cells, and delayed onset of diabetes. In mice, exposure to anti-serpinB13 mAb alone also decreased islet inflammation, and coadministration of this reagent and a suboptimal dose of anti-CD3 mAb accelerated recovery from diabetes. Czyzyk et al., Enhanced Anti-Serpin Antibody Activity Inhibits Autoimmune Inflammation in Type 1 Diabetes, Journal of Immunology, 2012, 188:6319-6327. It has also been observed that injecting anti-serpin B13 monoclonal Ab enhanced beta cell proliferation and Reg gene expression, induced the generation of ˜80 pancreatic islets per animal, and ultimately led to increase in the beta cell mass. These findings are relevant to human TID because the analysis of subjects recently diagnosed with TID revealed an association between baseline anti-serpin activity and slower residual beta cell function decline in the first year after the onset of diabetes. Kryvalap et al., Antibody Response to Serpin B13 Induces Adaptive Changes in Mouse Pancreatic Islets and Slows Down the Decline in the Residual Beta Cell Function in Children with Recent Onset of Type 1 Diabetes Mellitus, JBC, 291 (1): 266-278 (Jan. 1, 2016). It has been observed that cellular proliferation in mouse and human pancreatic islets is regulated by serpin B13 inhibition and downstream targeting of E-cadherin by cathepsin L. Lo et al., Diabetologia (2019) 62:822-834.

Thus, there is a continuing need for compositions and methods for the treatment of OVA-serine proteinase inhibitor (serpin) B13-related disorders in humans.

### SUMMARY

The present disclosure is based, at least in part, on the development of new monoclonal antibodies that selectively and specifically bind to OVA-serine proteinase inhibitor (serpin) B13. These antibodies and antigen-binding fragments thereof are useful for inhibiting serpin B13 and for treating serpin B13-related diseases, e.g., type I diabetes. Provided herein are these antibodies and antigen-binding fragments thereof, compositions and kits containing these antibodies and antibody fragments, and various methods of using these antibodies and antigen-binding fragments.

The new antibodies or antigen-binding fragments thereof have anti-serpin B13 effects. In some embodiments, the new antibodies or antigen-binding fragments thereof are chimeric antibodies. In some embodiments, the new antibodies or antigen-binding fragments thereof are humanized. The antigen-binding fragments can be Fab fragments, F(ab′)₂ fragments, scFv fragments, or diabodies.

In another general aspect, the disclosure includes compositions that include at least one isolated monoclonal antibody or antigen-binding fragment disclosed herein.

In yet another aspect, the disclosure includes methods of inhibiting serpin B13 and methods of treating serpin B13-related disorders in a subject, e.g., a human, as well as uses of the compositions described herein to treat such serpin B13-related disorders. The methods of inhibiting serpin B13 in a subject include administering to the subject an effective amount of one or more of the compositions disclosed herein. The methods of treating a serpin B13-related disorder in a subject include first identifying a subject that has an serpin B13-related disorder; and then administering to the subject an effective amount of a monoclonal antibody described herein, e.g., one that binds to serpin B13. The monoclonal antibodies disclosed herein can be administered by various routes, e.g., intravenously, intradermally, subcutaneously, or orally.

In some embodiments, the new monoclonal antibodies disclosed herein are used to treat diabetes, such as type I diabetes, type 2 diabetes and diabetes in patients with chronic pancreatitis who undergo total pancreatectomy with autologous islet transplantation and still remain insulin dependent. In some embodiments, the new monoclonal antibodies disclosed herein are used to treat a serpin B13-related disorder, wherein the disorder is inflammatory or central nervous system disease. In some embodiments, the new monoclonal antibodies disclosed herein are used to treat bone fracture, skin wound/ulcer healing including diabetic foot, hair loss, multiple sclerosis, or lupus.

In some embodiments, the isolated monoclonal antibodies or antigen-binding fragments thereof (1) bind to serpin B13, and (2) comprise a heavy chain CDR1, a heavy chain CDR2, and a heavy chain CDR3. The heavy chain CDR1 can comprise the amino acid sequence of SEQ ID NO: 1, 2, 26, 60, 70 or 80 or the amino acid sequence of SEQ ID NO:1, 2, 26, 60, 70 or 80 with a substitution at one, two, or three amino acid positions. The heavy chain CDR2 can comprise the amino acid sequence of SEQ ID NO:4, 27, 61, 71 or 81 or the amino acid sequence of SEQ ID NO: 4, 27 61, 71 or 81 with a substitution at one, two, or three amino acid positions. The heavy chain CDR3 can comprise the amino acid sequence of SEQ ID NO:6, 28, 62, 72 or 82 or the amino acid sequence of SEQ ID NO:6, 28, 62, 72 or 82 with a substitution at one, two, or three amino acid positions. In some embodiments, the isolated monoclonal antibodies or antigen-binding fragments can further include one or more of the following light chain CDRs: (1) a light chain CDR1 comprises the amino acid sequence of SEQ ID NO:8, 29, 63, 73 or 83 or the amino acid sequence of SEQ ID NO:8, 29, 63, 73 or 83 with a substitution at one, two, or three amino acid positions; (2) a light chain CDR2 comprises the amino acid sequence of SEQ ID NO:10, 64, 74 or 84, or the amino acid sequence of SEQ ID NO:10, 64, 74 or 84 with a substitution at one, two, or three amino acid positions; and (3) a light chain CDR3 comprises the amino acid sequence of SEQ ID NO: 12, 65, 75 or 85 or the amino acid sequence of SEQ ID NO: 12, 65, 75 or 85 with a substitution at one, two, or three amino acid positions.

In some embodiments, the heavy chain CDR1 comprises the amino acid sequence of SEQ ID NO:1.

In some embodiments, the heavy chain CDR1 comprises the amino acid sequence of SEQ ID NO:2.

In some embodiments, the heavy chain CDR1 comprises the amino acid sequence of SEQ ID NO:26.

In some embodiments, the heavy chain CDR1 comprises the amino acid sequence of SEQ ID NO:60.

In some embodiments, the heavy chain CDR1 comprises the amino acid sequence of SEQ ID NO:70.

In some embodiments, the heavy chain CDR1 comprises the amino acid sequence of SEQ ID NO:80.

In certain embodiments, the heavy chain CDR2 comprises the amino acid sequence of SEQ ID NO:4.

In certain embodiments, the heavy chain CDR2 comprises the amino acid sequence of SEQ ID NO:27.

In some embodiments, the heavy chain CDR2 comprises the amino acid sequence of SEQ ID NO:61.

In some embodiments, the heavy chain CDR2 comprises the amino acid sequence of SEQ ID NO:71.

In some embodiments, the heavy chain CDR2 comprises the amino acid sequence of SEQ ID NO:81.

In certain embodiments, the heavy chain CDR3 comprises the amino acid sequence of SEQ ID NO:6.

In certain embodiments, the heavy chain CDR3 comprises the amino acid sequence of SEQ ID NO:28.

In certain embodiments, the heavy chain CDR3 comprises the amino acid sequence of SEQ ID NO:62.

In certain embodiments, the heavy chain CDR3 comprises the amino acid sequence of SEQ ID NO:72.

In certain embodiments, the heavy chain CDR3 comprises the amino acid sequence of SEQ ID NO:82.

In certain embodiments, the light chain CDR1 comprises the amino acid sequence of SEQ ID NO:8.

In certain embodiments, the light chain CDR1 comprises the amino acid sequence of SEQ ID NO:29.

In certain embodiments, the light chain CDR1 comprises the amino acid sequence of SEQ ID NO:63.

In certain embodiments, the light chain CDR1 comprises the amino acid sequence of SEQ ID NO:73.

In certain embodiments, the light chain CDR1 comprises the amino acid sequence of SEQ ID NO:83.

In certain embodiments, the light chain CDR2 comprises the amino acid sequence of SEQ ID NO:64.

In certain embodiments, the light chain CDR2 comprises the amino acid sequence of SEQ ID NO:74.

In certain embodiments, the light chain CDR2 comprises the amino acid sequence of SEQ ID NO:84.

In certain embodiments, the light chain CDR3 comprises the amino acid sequence of SEQ ID NO:65.

In certain embodiments, the light chain CDR3 comprises the amino acid sequence of SEQ ID NO:75.

In certain embodiments, the light chain CDR1 comprises the amino acid sequence of SEQ ID NO:85.

In some embodiments, the one, two, or three amino acid substitutions are conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Conservative amino acid substitutions typically include substitutions within the same family.

In some embodiments, the isolated monoclonal antibodies or antigen-binding fragments thereof are a humanized antibody. In certain embodiments, the heavy chain comprises the amino acid sequence of SEQ ID NO:18. In certain embodiments, the light chain comprises the amino acid sequence of SEQ ID NO:20.

In some embodiments, the isolated monoclonal antibodies or antigen-binding fragments thereof are a human recombinant antibody. In certain embodiments, the heavy chain comprises the amino acid sequence of SEQ ID NO: 31, SEQ ID NO:41 or SEQ ID NO:49. In certain embodiments, the light chain comprises the amino acid sequence of SEQ ID NO:37, SEQ ID NO: 45 or SEQ ID NO:53.

In certain embodiments, the heavy chain variable region comprises the amino acid sequence of SEQ ID NO: 57, SEQ ID NO:67 or SEQ ID NO:77. In certain embodiments, the light chain variable region comprises the amino acid sequence of SEQ ID NO:59, SEQ ID NO: 69 or SEQ ID NO:79.

In some embodiments, the antigen-binding fragments can be a Fab fragment, an F(ab′)₂ fragment, a scFv fragment, or a sc(Fv)₂ diabody.

In some embodiments, the monoclonal antibodies and antigen-binding fragments disclosed herein bind to serpin B13 with an affinity of about 1 nM to about 8 nM. In certain embodiments, the monoclonal antibodies and antigen-binding fragments disclosed herein bind to serpin B13 with an affinity of about 1 nM to about 2 nM (e.g., 1.21 nM).

In some embodiments, the isolated monoclonal antibodies or antigen-binding fragments also bind to serpin B13. As used herein, the term “monoclonal antibody” refers to a population of antibody molecules that contain only one species of an antigen binding site capable of immune-reacting with a particular epitope of a polypeptide or protein. A monoclonal antibody thus typically displays a single binding affinity for the protein to which it specifically binds.

As used herein, the term “chimeric antibody” refers to an antibody that has been engineered to comprise at least one human constant region. For example, one or all (e.g., one, two, or three) of the variable regions of the light chain(s) and/or one or all (e.g., one, two, or three) of the variable regions the heavy chain(s) of a mouse antibody (e.g., a mouse monoclonal antibody) can each be joined to a human constant region, such as, without limitation an IgG1 human constant region. In certain embodiments, the isolated monoclonal antibody or antigen-binding fragment is a chimeric antibody wherein the heavy chain comprises the amino acid sequence of SEQ ID NO:22. In certain embodiments, the isolated monoclonal antibody or antigen-binding fragment is a chimeric antibody wherein the light chain comprises the amino acid sequence of SEQ ID NO:24.

“Fragment” or “antibody fragment” as the terms are used herein refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full-length antibody polypeptide, although the term is not limited to such cleaved fragments.

“Humanized antibody,” as the term is used herein, refers to an antibody that has been engineered to comprise one or more human framework regions in the variable region together with non-human (e.g., mouse, rat, or hamster) complementarity-determining regions (CDRs) of the heavy and/or light chain. In some embodiments, a humanized antibody comprises sequences that are entirely human except for the CDR regions. Humanized antibodies are typically less immunogenic to humans, relative to non-humanized antibodies, and thus offer therapeutic benefits in certain situations.

As used herein, the term “percent sequence identity” refers to the degree to which any given query sequence is the same as a subject sequence. Percentage of “sequence identity” is determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. It is noted that a query nucleotide or amino acid sequence that aligns with a subject sequence can result in many different lengths, with each length having its own percent identity.

The term “therapeutic treatment” or “treatment” means the administration of one or more pharmaceutical agents to a subject or the performance of a medical procedure on the body of a subject (e.g., surgery, such as organ transplant or heart surgery). The term therapeutic treatment also includes an adjustment (e.g., increase or decrease) in the dose or frequency of one or more pharmaceutical agents that a subject can be taking, the administration of one or more new pharmaceutical agents to the subject, or the removal of one or more pharmaceutical agents from the subject's treatment plan.

As used herein, a “subject” is an animal, e.g., a mammal, e.g., a human, monkey, dog, cat, horse, cow, pig, goat, rabbit, or mouse.

An “effective amount” is an amount sufficient to effect beneficial or desired results. For example, a therapeutically effective amount is one that achieves the desired therapeutic effect. An effective amount can be administered in one or more administrations, applications or dosages. A therapeutically effective amount of a pharmaceutical composition (i.e., an effective dosage) depends on the pharmaceutical composition selected. The compositions can be administered from one or more times per day to one or more times per week, including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the pharmaceutical compositions described herein can include a single treatment or a series of treatments.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A-1I together provide the nucleic acid sequences of B29_graft heavy chain (SEQ ID NO: 91).

FIGS. 2A-2H together provide the nucleic acid sequences of B29_graft light chain (SEQ ID NO: 92).

FIGS. 3A-3I together provide the nucleic acid sequences of B29_chimeric heavy chain (SEQ ID NO: 93).

FIGS. 4A-4H together provide the nucleic acid sequences of B29_chimeric light chain (SEQ ID NO: 94).

FIGS. 5A and 5B. The recipient of B29_H (SEQ ID NO: 14) is human germline IGHV3_66*01. The sequence after CDR-grafted is named B29_graft_H (SEQ ID NO: 95). The sequence alignment of the germline gene before and after grafting is shown in FIG. 5A. Red color indicates the CDR region. The darker color indicates the more identical sequences. The recipient of B29_L (SEQ ID NO: 16) is human germline IGKV1_39*01. The antibody sequence after CDR-grafted is named B29_graft_L (SEQ ID NO: 96). The sequence alignment of the germline gene before and after grafting is shown in FIG. 5B. Pink color indicates the CDR region. The darker color indicates the more identical sequences.

FIGS. 6A-6C. Impact of mouse monoclonal antibody to serpin B13 (clone B29) on tissue regeneration in different organs. (FIG. 6A) Bone healing. B6 Mice (n=4/group) were subjected to tibia fracture on day zero and intraperitoneally injected 4 times with 50 μg of B29 (or IgG control) over one-week period. Mice were X-rayed on day 9, 14 and 21 to determine the size (volume) of bone callus. A representative image from each group is shown from analysis day 21 (right) as well as the average for all animals/group from the analysis on days 9, 14 and 21 (left). (FIG. 6B) Skin ulcer healing. B6 mice (n=3/group) were injected at the base of tail with 100 μL of CFA and 7 days later injected four times with B29 or IgG control (100 μg per injection). Mice were weekly followed for two months and their tails were photographed by the end of the study. One representative image from each group is shown. (FIG. 6C) Mortality rates in the experimental autoimmune encephalomyelitis (EAE). B6 mice (n=14/group) were immunized with 100 μg of pMOG antigen in Complete Freund's adjuvant (CFA) on day 0, and then again on day 7 with the same antigen in incomplete adjuvant. Immunized mice were intraperitoneally injected 4 times with 100 μg of B29 (or IgG) on days 7, 8, 11 and 13. Mice were observed daily for 31 days for limb paralysis and mortality.

FIGS. 7A and 7B. Impact of baseline serpinB13 AAs on beta cell function in recent onset TID patients. The placebo subjects previously recruited to Type 1 Diabetes TrialNet protocols TN02 MMF/DZB, TN08 GAD-Alum Vaccine, TN09 CTLA-4 or TN14 Anti-IL1b were examined. The impact of baseline serpin response was examined with regard to C-peptide, either fasting (A) or stimulated at 90 min. during mixed-meal tolerance test (MMTT) (B). Linear regression was used for the analysis.

FIGS. 8A-8F. A mAb to serpinB13, clone B29, promotes development of pancreatic Ngn3+ endocrine progenitor cells and helps to prevent severe diabetes. (A) A schematic of the experiment. Healthy Balb/c females were i.p. injected four times with 50 μg of B29 (αB13), or control IgG, during mid pregnancy. The pancreatic tissue in the offspring was examined on embryonic day E14.5 and E16.5, at birth (P0) (B and C), or in adulthood at 12 weeks of age following induction of diabetes with STZ at 8 weeks of age (D-F). (B-C) Quantitative analysis of Ngn3+ cells by I.F microscopy (B; insert in B shows the impact of CatL deficiency), and flowcytometry analysis of active Notch intracellular domain (NICD) using Val1744 polyclonal antibody (C), in embryonic pancreases. The right panel in C shows the average mean fluorescence channel of antibody staining from three independent experiments. (D-F) A follow-up of diabetic mice with gestational exposure to anti-serpinB13 mAb. The quantitative analysis is shown for the residual β-ell mass at 4 weeks after STZ injection (D), random blood glucose at 1, 2, 3, and 4 weeks after STZ injection (E), and serum creatinine at 4 weeks after STZ injection (F). *p<0.05, **p<0.01, and ***p<0.001.

FIGS. 9A-9M. SerpinB13 impedes development of endocrine progenitor cells in the pancreas. (A) Microscopic images of serpinB13 expression in the embryonic pancreas. (B) Quantitative analysis by ELISA of serpinB13 release into the extracellular environment in vitro. The supernatants were collected and examined after 48 hours of culturing E12.5 explants of the pancreas (3 combined embryonic rudiments per well, n=6), heart (n=7) or media alone (n=7), two independent experiments. (C to E) Impact of serpinB13 on Ngn3⁺ cells in vitro. (C) A schematic of the experiment. (D) Number of Ngn3⁺ cells after 72 hours of incubation with recombinant serpinB13 of mouse (rmB13, n=8) or human (rhB13, n=10), chicken ovalbumin (OVA, n=6) (10 μg/mL each), buffer control (buffer, n=8), or control Ab (n=6) and anti-serpinB13 mAb (aB13, n=5) (both antibodies at 1 μg/mL), two independent experiments. (E) Representative microscopic images of data shown in (D). (F to H) Impact of inhibition of serpinB13 on Ngn3⁺ cells in vivo. (F) A schematic of the experiment. (G) The quantitative analysis by IF microscopy of Ngn3⁺ cells (following injection of anti-serpinB13 mAb into pregnant mothers) in embryonic pancreases isolated at day E14.5 (control Ab/αB13, n=5), E16.5 (control Ab/αB13, n=5) and birth (OP) (control Ab, n=9; αB13, n=10), two independent experiments. (H) Representative microscopic images of data shown in (G). (I to K) The genetic lineage tracing of Ngn3⁺ cells in the pancreas following in vivo injections of anti-serpinB13 mAb. (I) A schematic of the experiment. (J) The quantitative analysis of insulin YFP cells (control Ab/αB13, n=4). (K) Representative microscopic IF images of data depicted in (J), two independent experiments. (L and M) Impact of inhibition of serpinB13 on insulin-positive area in E16.5 pancreas in vivo. (L) A quantitative analysis by IF microscopy of the area occupied by insulin-positive cells (control Ab, n=9; αB13, n=8), two independent experiments. (M) The representative microscopic IF images of data shown in (L). In (F) and (I), each pregnant mouse received one daily i.p injection of 50 μg of anti-serpinB13 mAb (or control antibody) on gestational day E10.5 through E13.5 (total dose 200 μg). Data are presented as the mean±SEM. One-way ANOVA with Dunnet's test (D), unpaired two-sided Student/test (G, J and L). Scale bars, 20 μm (A), 50 μm (E, H, K and M). NS, not significant. αB13, anti-serpinB13 mouse mAb.

FIGS. 10A-10J. Notch receptor-mediated repression of Ngn3⁺ cell development is controlled by the inhibitory function of serpinB13 on its protease target. (A to D) The quantitative analysis of Ngn3⁺ cells by IF microscopy. In (A), the wild-type (WT) and CatL-deficient (CatLKO) embryonic pancreases were isolated from pregnant mice at day E12.5, and after 48-hour culture of the explants, either alone or in the presence of anti-serpinB13 mAb or control Ab as indicated, they were harvested and processed for staining with anti-Ngn3 antibody. For (A left), n=6 (WT/CatLKO), six independent experiments. For (A right), n=4 (control Ab/αB13), four independent experiments. In (C), E12.5 WT pancreas explants were cultured for 48 hours with E64 protease inhibitor (10 μM) or DMSO solvent, as indicated (n=9 per group, three independent experiments). (B) and (D) show representative microscopic IF images of data depicted in (A) and (C), respectively. (E to J) Western blot analysis of the Notch 1 receptor ectodomain. The experimental approach to examine extra- and intracellular domains of intact (left) and cleaved by CatL (right) Notch1 is depicted in (E). The embryonic pancreas explants isolated at E12.5, were cultured with anti-serpinB13 mAb or control Ab at 1 μg/mL for 24 hours (F) (control Ab/αB13, n=6; six independent experiments) or 48 hours (G) (control Ab/αB13, n=4; four independent experiments), and examined for Notch 1. The representative Western blot images and the average of independent experiments are shown. Alternatively, E12.5 embryonic pancreas explants were treated for 48 hours (H, data from one of seven independent experiments are shown) or incubated with recombinant mouse CatL (rmCatL) at 2.5 μg/mL for the last 4 hours of the 48-hour incubation period (I, data from one of three independent experiments is shown), and assessed for the degradation fragments of the Notch 1 receptor. In (J), in addition to treatment with antibodies for 48 hours, the embryonic pancreas explants were treated with E64 (1 μM) or DMSO, as indicated (n=3 per group, three independent experiments). The representative Western blot image and the average of independent experiments are shown. For all Western blot analyses, the cells from three cultured embryonic pancreases were combined for each lane and examined using antibody raised against the N-terminal portion (Ala9-Gln526) of Notch1. Data are presented as the mean±SEM. Unpaired two-sided Student/test (A, C, F and G), one-way ANOVA with Sidak test (J). Scale bars, 20 μm. * (H, I, J) indicates cleavage fragments of Notch1. NS, not significant. αB13, anti-serpinB13 mouse mAb.

FIGS. 11A-11N. Inhibition of serpinB13 during embryogenesis results in a greater expansion of β-cell mass and prevents severe diabetes in adulthood. (A to F) A follow up of healthy mice with gestational exposure to anti-serpinB13 mAb. (A) A schematic of the experiment. The quantitative analysis of the islet number (B), endocrine cell number (C), and representative flow cytometry scatter plots (D) (control Ab, n=11; αB13, n=10; three independent experiments). (E) Quantification of β-cell mass. (F) Representative microscopic IF images of data depicted in (E) (control Ab, n=4; αB13, n=4; two independent experiments). (G to N) A follow up of diabetic mice with gestational exposure to anti-serpinB13 mAb. (G) A schematic of the experiment. (H) Quantification of the residual β-cell mass at 4 weeks after STZ injection (150 mg/kg). (I) Representative microscopic IF images of data depicted in (H). (J) Glucose tolerance test (ipGTT) and (K) AUC at 2 weeks after streptozotocin (STZ) injection. (L) Random blood glucose levels at 1, 2, 3, and 4 weeks after STZ injection. (M) Serum creatinine 4 weeks after STZ injection. (N) Body weight at 1, 2, 3 and 4 weeks after STZ injection (for G to N, control Ab, n=11; αB13, n=10; two independent experiments). Data are presented as mean±SEM. Unpaired two-sided Student/test (B, C, E, H, K, M) and two-way ANOVA with Sidak test (J, L, N). Scale bars, 1 mm. NS, not significant. αB13, anti-serpinB13 mouse mAb; AUC, area under curve.

FIGS. 12A-12L. Impact of autoantibodies to serpinB13 on progression to type 1 diabetes in humans and pancreatic Ngn3⁺ cell output during embryogenesis. (A) The frequency of serpinB13 AAs in human subjects with low (n=69), modest (n=69), intermediate (n=70) and high (n=70) risk levels for TID. (B) Incidence of diabetes and (C) time to diabetes onset in high and intermediate risk groups combined (n=140), either negative (n=102) or positive (n=38) for serpinB13 AA. (D) Quantitative analysis by IF microscopy of Ngn3⁺ cells in mouse embryonic pancreas explants (E12.5) incubated in vitro for 48 hours with individual human serum samples from Diabetes Prevention Trial for Type 1 Diabetes (DPT-1) enrolled subjects, either negative (serpinB13 AA″, n=9) or positive (serpinB13 AA, n=8) for serpinB13 AA; three independent experiments. (E) Representative microscopic IF images of data shown in (D). (F) Cleavage of the substrate by CatL in the presence of serpinB13 and human mAb to serpinB13 (hαB13) or control human antibody (h.Control Ab) over time. The average of three independent experiments (left) and quantification of their AUC (right) is shown. (G) Quantitative analysis by IF microscopy of Ngn3⁺ cells in mouse embryonic pancreas explants (E12.5) incubated in vitro for 48 hours with serpinB13 AA-negative human serum samples from DPT-1 subjects, which were reconstituted with hαB13 at 10 μg/mL (n=9) or h.Control Ab (n=9); three independent experiments. (H). Representative microscopic IF images of data shown in (G). (I) Experimental approach to deplete DPT-1 sera of serpinB13 AA. (J) Quantitative analysis by IF microscopy of Ngn3⁺ cells in mouse embryonic pancreas explants (E12.5) incubated in vitro for 48 hours with human serum samples from DPT-1 subjects, which were depleted for serpinB13 AA (n=6) or sham depleted (n=6); two independent experiments. Both positive (left) and negative (right) serum samples examined. (K) Representative microscopic IF images of data shown in (K). (L) A model of spatial relationship between serpinB13, cathepsin L and antibody response, and its influence on progression to TID. Data are presented as the mean±SEM. Log-rank (Mantel-Cox) test, hazard ratio 1.898 (C); unpaired two-sided Student/test (D); unpaired one-sided Student/test (G); one-way Anova with Sidak test (F-right, J) (F). Scale bars, 100 μm. NS, not significant. αB13, anti-serpinB13 mouse mAb; hαB13, anti-serpinB13 human recombinant antibody; rhB13, recombinant human serpinB13; h.Control Ab, recombinant human IgG1 isotype control; #, fluorescent signal reading limit; AUC, area under curve.

FIG. 13. Flow cytometry analysis of serpinB13 expression in the pancreatic epithelium during embryonic development. Representative flow cytometry scatter plots are shown for the pancreas and heart isolated from embryos at gestational day E16.5. CD31⁻CD45⁻ cell suspensions were stained with antibodies against epithelial marker (anti-EpCAM) and serpinB13 (clone B29).

FIGS. 14A-14C. Quantitative analysis of Ngn3⁺ cells (in vitro). (A) Flow cytometry analysis of Ngn3⁺ cells. The Balb/c pancreatic explants at embryonic day E12.5 were cultured in vitro with anti-serpinB13 mAb (or control Ab) at 1.0 μg/mL for 48 hours, and then examined for the number of Ngn3⁺ and CK19⁺ cells. Data are displayed both in absolute numbers (left) and as the percentages of CK19⁺ cells that express Ngn3 (right). (B) Representative flow cytometry scatter plots of data shown in (A). (C) Quantitative analysis by flow cytometry of CK19⁺ cells. For (A) and (C), n=10 (control Ab); n=9 (αB13), three independent experiments. Data are presented as the mean±SEM. Unpaired two-sided Student/test. NS, not significant. αB13, anti-serpinB13 mouse mAb.

FIGS. 15A-15C. Changes in Ngn3⁺ cell number following inhibition of serpinB13 in embryonic pancreas explants cultured in vitro. (A and B) Quantitative analysis by IF microscopy. The pancreas explants at embryonic day E12.5 were cultured in vitro with varying concentrations of anti-serpinB13 mAb (or control Ab) for 48 hours, as indicated. The pancreatic sections were then analyzed for the number of Ngn3⁺ cells (A) and the percentage of the total area occupied by CK19⁺ cells per explant (B). n=7 (control Ab/0.050); n=8 (αB13/0.050); n=5 (control Ab/0.500); n=7 (αB13/0.500); two independent experiments. (C) Representative microscopic IF images of data shown in (A) (upper panel) and (B) (lower panel). Data are presented as the mean±SEM. Two-way ANOVA with Sidak test. Scale bars, 50 μm. NS, not significant. αB13, anti-serpinB13 mouse mAb.

FIGS. 16A-16C. Western blot analysis of Ngn3 expression in vivo. (A) A schematic of the experiment. Pregnant Balb/c females received one daily i.p injection of 50 μg of anti-serpinB13 mAb (or control Ab) on gestational day E10.5 through E13.5 (total dose 200 μg). On day E16.5, embryonic pancreases were isolated and directly used for analysis. (B) Representative Western blot. (C) Densitometry analysis of Western blots. n=11 (control Ab/αB13), five independent experiments. Data are presented as the mean±SEM. Unpaired two-sided Student/test. αB13, anti-serpinB13 mouse mAb.

FIGS. 17A-17C. Expression of serpinB13 and the impact of binding to distinct monoclonal antibodies on the number of Ngn3⁺ pancreatic endocrine progenitor cells at birth. (A) Quantitative analysis by IF microscopy of Ngn3⁺ cells. Pregnant Balb/c female mice were injected i.p. with anti-serpinB13 mAbs (either clone B29 or B34), or control Ab, exactly as described in FIG. 1F. The number of Ngn3⁺ cells in the pancreas of the newborn pups (OP) was assessed. n=9 for each group, two independent experiments. (B) The representative microscopic IF images of data shown in (A). (C) Microscopic images of IF staining of the skin and the pancreas isolated from adult mice with antiserpinB13 mAbs or control Ab, as indicated. Data are presented as the mean±SEM. Ordinary one-way ANOVA with Dunnett test. Scale bars, 50 μm (b), 20 μm (c). NS, not significant. αB13, anti-serpinB13 mouse mAb.

FIGS. 18A-18C. Genetic tracing of Ngn3⁺ cells in the pancreas of healthy and diabetic adult mice treated with anti-serpinB13 mAb. (A) The protocol of mice treatment with streptozotocin (STZ), tamoxifen (TAM), and anti-serpinB13 mAb (or control Ab). Eight-week old Ngn3Cre^{ERT}R26^{YFP} mice were induced into diabetes with STZ (150 mg/kg) and genetically labelled with YFP to mark Ngn3⁺ cells, using TAM one day after injection of STZ, and an additional three times every other day thereafter. The antibodies were injected every day for 7 days at 100 μg/injection during the first week after STZ treatment. (B) Quantitative analysis by IF microscopy of YFP cells expressing insulin at two weeks following induction of diabetes with STZ. Note that a diabetic state was necessary to demonstrate an increase in the number of double-positive cells following inhibition of serpinB13 with mAb. n=5 (groups treated with STZ), n=4 (groups without STZ), the average of two independent experiments. (C) Representative microscopic IF images of YFP insulin cells depicted in (B). Data are presented as the mean±SEM. Ordinary one-way ANOVA with Sidak test. Scale bars, 10 μm. NS, not significant. αB13, anti-serpinB13 mouse mAb.

FIGS. 19A-19B. Examination of the extracellular domain of Notch1 receptor. (A) Flow cytometry analysis. Anti-serpinB13 mAb (or control Ab) was injected daily into pregnant Balb/c females for four days during gestation (day E10.5 through E13.5, 50 μg/injection), and on day E15.5 the embryonic pancreases were isolated and examined. Single cell suspensions were stained with a mAb that recognizes the extracellular portion of Notch1 (clone 22E5) and analyzed by FACS. The bars represent the average MFC of antibody staining. n=13 (control Ab); n=14 (αB13); three independent experiments. (B) A representative flow cytometry histogram of data shown in (A). Data are presented as the mean±SEM. Unpaired two-sided Student/test. αB13, anti-serpinB13 mouse mAb. MFC, mean fluorescent channel.

FIGS. 20A-20C. Impact of cathepsin L on the number of Ngn3⁺ cells. (A) A schematic of the experiment. The embryonic pancreas explants isolated at day E12.5 of gestation, were cultured in media for the first 24 hours, followed by recombinant mouse cathepsin L at 2.5 μg/mL (rmCatL, n=7) or pH-buffer control (pH 6.0) (buffer, n=8) for 4 hours. The pancreas explants were then washed, incubated for an additional 48 hours, and harvested for analysis. (B) Quantitative analysis by IF microscopy of Ngn3⁺ cells. The average of three independent experiments is shown. (C) A representative microscopic IF images of data depicted in (B). Data are presented as the mean±SEM. Unpaired, two-sided Student/test. Scale bars, 100 μm.

FIGS. 21A-21C. Examination of the intracellular domain of the Notch1 receptor. (A) Western blot analysis. The embryonic pancreases isolated at day E12.5 of gestation from Balb/c females were cultured with anti-serpinB13 mAb (n=7) or control Ab (n=7) at 1 μg/mL for 48 hours and harvested. Three culture harvests were combined for Western blotting with mAb recognizing the intracellular portion of Notch1 (clone D1E11). The bars represent the average of seven independent experiments by densitometry analysis. (B and C) Flow cytometry analysis. Anti-serpinB13 mAb (or control Ab) was injected daily into pregnant Balb/c female mice, from day E10.5 through to E13.5 (50 μg/injection). On day E15.5 of gestation the embryonic pancreases were isolated and examined. n=11 (control Ab), n=9 (αB13), two independent experiments (B). Alternatively, the antibodies were directly added to E12.5 embryonic pancreas explants and cultured for 48 hours in vitro. n=3 (control Ab), n=4 (αB13), two independent experiments (C). The single cell suspensions (B and C) were stained with the polyclonal antibody, Val1744, which recognizes the active Notch intracellular domain (aNICD) that is cleaved by the γ-secretase complex. Representative flow cytometry histograms (B left, C left), and the average MFC of antibody staining (B right, C right), are shown. Data are presented as the mean±SEM.

Unpaired, two-sided Student/test. αB13, anti-serpinB13 mouse mAb. MFC, mean fluorescent channel.

FIGS. 22A-22B. Analysis of Notch1 gene expression. (A) Embryonic E12.5 mouse pancreatic explants or (B) pregnant at E10.5 Balb/c mouse females were treated with anti-serpinB13 mAb (or control Ab), exactly as described in the schematics and legend to FIG. 1. The tissues were harvested and examined in biological duplicates or triplicates by quantitative PCR for expression of Notch 1 gene. In (B lower), the analysis was performed on sorted viable (EPCAM⁺ CD45⁺ CD31⁻7AAD⁻) epithelial cells. The bars represent the average of three (A lower) and two (B lower) independent experiments. Unpaired Student's/test was used for the analysis. NS, not significant. αB13, anti-serpinB13 mouse mAb.

FIGS. 23A-23B. The pancreas and body weight in mice with exposure to anti-serpinB13 mAb during pregnancy and embryonic life. (A) The animals were injected with anti-serpinB13 mAb (or control Ab) during mid pregnancy, exactly as depicted in FIG. 3A, and followed for the weight of their body at 64 days of age (P64), and the pancreas at the same time (n=11 (control Ab), n=14 (αB13), two independent experiments), and at birth (P0) (n=5 per group, two independent experiments). (B) The pregnant mothers were injected with anti-serpinB13 mAb (or control Ab) during mid pregnancy, exactly as depicted in FIG. 3A, and followed for their body weight on a delivery day (n=6 (control Ab), n=7 (αB13), two independent experiments, as well as the body weight of their newborn pups (P0) (n=45 (control Ab), n=55 (αB13), two independent experiments). Data are presented as the mean±SEM Unpaired Student/test was used for the analysis. NS, not significant. αB13, anti-serpinB13 mAb.

FIGS. 24A-24B. The severity of diabetes in adult mice following exposure to anti-serpinB13 mAb during embryonic life. Eight-week old Balb/c mice that received control Ab (A) or anti-serpinB13 mAb (B) during gestation were induced into a diabetic state with streptozotocin (STZ) during adulthood, and followed for diabetes according to the protocol depicted in FIG. 3G. The data are shown for mice at 4 weeks after STZ injection and displayed as the percentages of mice with distinct ranges of random blood glucose levels, as indicated. n=11 (control Ab); n=10 (αB13). αB13, anti-serpinB13 mouse mAb.

FIG. 25. The specificity of anti-serpin activity. Three recombinant human anti-serpinB13 antibodies were examined in the Luminex-based assay for binding to other clade B serpins, in order as indicated. The final concentration of each antibody was 1.0 μg/mL. The data are expressed as immunofluorescence intensity measured for binding to individual human serpin antigens after subtracting the background. The latter was determined as the average antibody binding to Luminex beads coated with Gfp and Scgn control antigens. Similar data were obtained for antigen binding of Fab fragments of these antibodies (data not shown).

FIGS. 26A-26B. The dose-dependent control of Cathepsin L activity by recombinant human serpinB13 and antibody influence on substrate cleavage. (A) Inhibition of a substrate cleavage by CatL with different doses of recombinant human serpinB13 over time. The average of three independent experiments (left) and quantification of their areas under the curves (AUC) (right) is shown. (B) The substrate cleavage by CatL in a presence of either human mAb to serpinB13 (hαB13) or control human Ab (h.Control Ab) over time. The average of three independent experiments (left) and quantification of their areas under the curves (AUC) (right) is shown. Data are presented as the mean±SEM. One-way Anova with Sidak test (A right and B right). NS, not significant; hαB13, recombinant human anti-serpinB13 antibody; rhB13, recombinant human serpinB13; h.Control Ab, recombinant human Isotype control antibody; #, fluorescent signal readings limit; AUC, area under the curve.

FIGS. 27A-27C. Recombinant fully human antibody sequences were developed. The CDR Analysis for clone 1 is provided in FIG. 27A. The CDR Analysis for clone 2 is provided in FIG. 27B. The CDR Analysis for clone 3 is provided in FIG. 27C.

### DETAILED DESCRIPTION

Proteases are ubiquitously expressed in the body and they play a critical role such as cell differentiation, proliferation, apoptosis and other processes. Protease activity is tightly controlled by a number of inhibitors, some of which are known as Serpins. One particular serpin molecule is Serpin B13. Serpin B13 is primarily expressed in the epithelial cells, and its main feature is to block Cathepsin L protease. The present disclosure is based, at least in part, on the development of new monoclonal antibodies that selectively and specifically bind to serpin B13. These antibodies and antigen-binding fragments thereof are useful for inhibiting serpin B13 and for treating serpin B13-related diseases, e.g., type I diabetes. Provided herein are these antibodies and antigen-binding fragments thereof, compositions and kits containing these antibodies and antibody fragments, and various methods of using these antibodies and antigen-binding fragments.

The term monoclonal antibody refers to a population of antibody molecules that contain only one species of an antigen binding site capable of immune-reacting with a particular epitope of a polypeptide or protein. A monoclonal antibody thus typically displays a single binding affinity for the protein to which it specifically binds.

In general, a given antibody can include one of five different types of heavy chains: alpha, delta, epsilon, gamma, and mu, which have different amino acid sequences in the constant region. These different types of heavy chains give rise to five classes of antibodies: IgA (including IgA1 and IgA2), IgD, IgE, IgG (IgG1, IgG2, IgG3, and IgG4) and IgM, respectively. An antibody also comprises one of two types of light chains: kappa or lambda, which differ in the amino acid sequence of the light chain constant domains. IgG, IgD, and IgE antibodies generally contain two identical heavy chains and two identical light chains, and contain two antigen combining domains, each composed of a heavy chain variable region (V_{H}) and a light chain variable region (V_{L}).

Antigen-binding fragments include any antibody fragments containing the active binding region of the antibody, such as a Fab fragment, a F(ab′)₂ fragment, or a single-chain Fv (scFv) fragment. Such fragments can be produced from the antibody using techniques well established in the art. For example, the F(ab′)₂ fragments can be produced by pepsin digestion of the antibody molecule, and the Fab fragments can be generated by reducing the disulfide bridges of the F(ab′)₂ fragments.

ScFv antibodies are single-chain polypeptides produced by linking V_{L} and a V_{H} via a linker or such (see, e.g., Bird et al., Science, 242 (4877): 423-426 (1988)). The heavy chain variable region and light chain variable region of an scFv may be derived from any antibody described herein. The peptide linker for linking the variable regions is not particularly limited. For example, an arbitrary single-chain peptide containing about three to 25 residues can be used as the linker. A “diabody” is a noncovalent dimer of single-chain Fv (scFv) fragment that consists of the heavy chain variable (V_{H}) and light chain variable (V_{L}) regions connected by a small peptide linker. Another form of diabody is where two scFv fragments are covalently linked to each other. In general, the linker is short enough such that the V_{L} and a V_{H} cannot bind to each other in the dimer. In certain embodiments, the number of amino acid residues constituting the linker is, for example, about five residues. Thus, the V_{L} and a V_{H} encoded on the same polypeptide cannot form a single-chain variable region fragment and will form a dimer with another single-chain variable region fragment. As a result, the diabody has two antigen binding sites.

### Antibodies and Antibody Fragments

Provided herein are novel monoclonal antibodies and antigen-binding fragments that bind to serpin B13. As known in the art, an antibody's specificity towards a given antigen is mediated by the heavy and light chain variable regions. In particular, the specificity of an antibody towards a given antigen is primarily determined by short sequences within the heavy and light chain variable regions called complementarity determining regions (CDRs). Provided herein are the nucleotide and amino acid sequences of the heavy and light chain variable regions and the heavy and light chain CDRs of the anti-serpin B13 antibodies and antibody fragments.

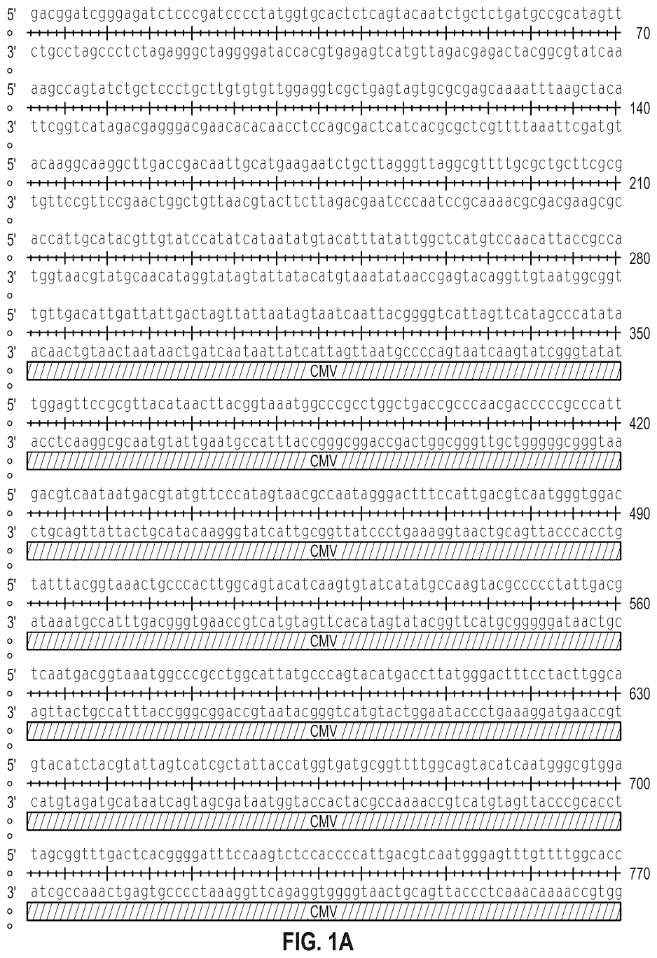

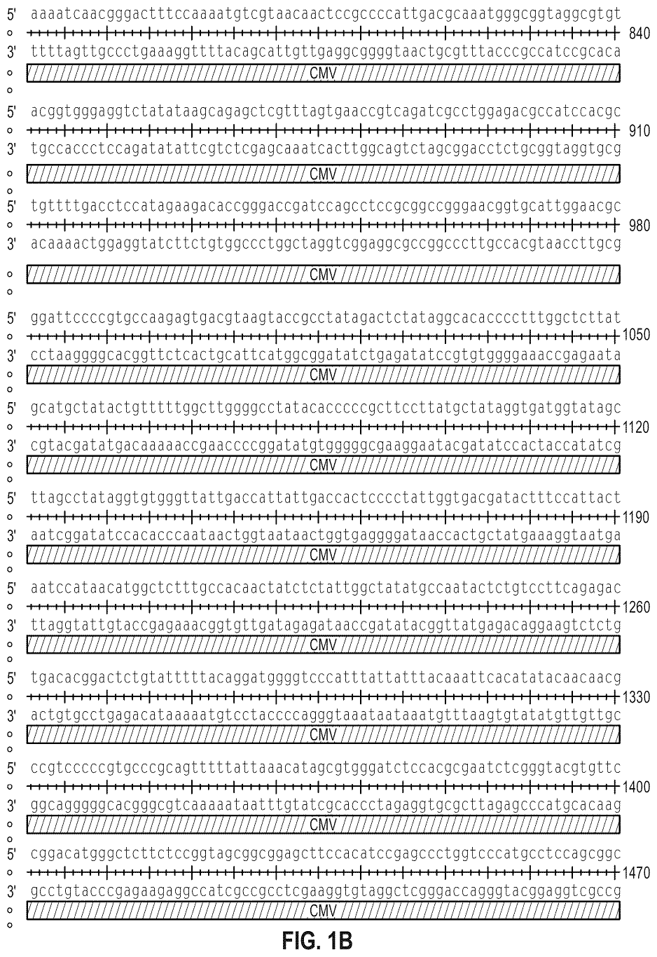

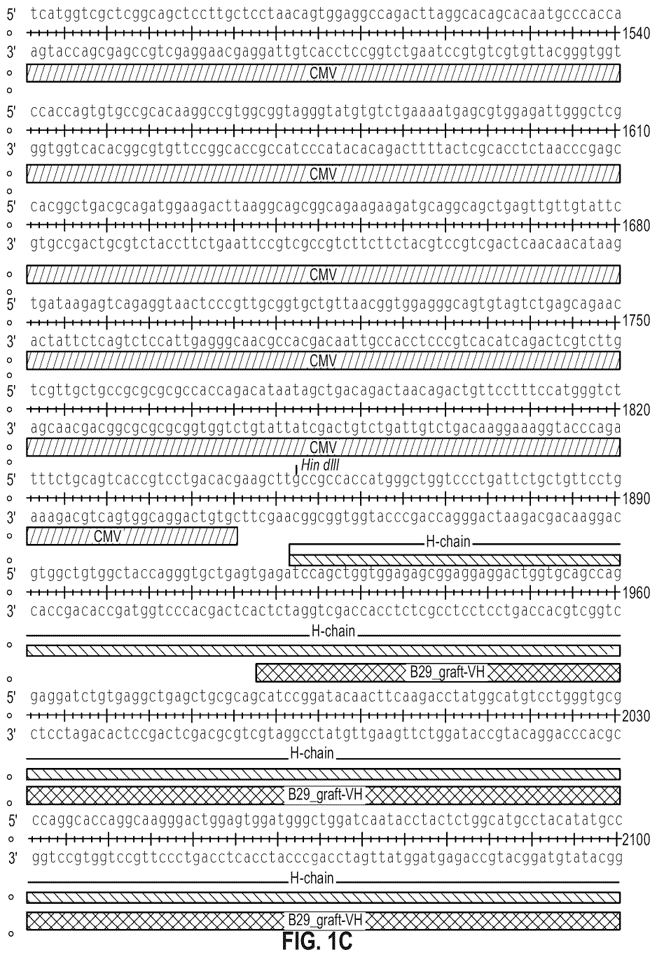

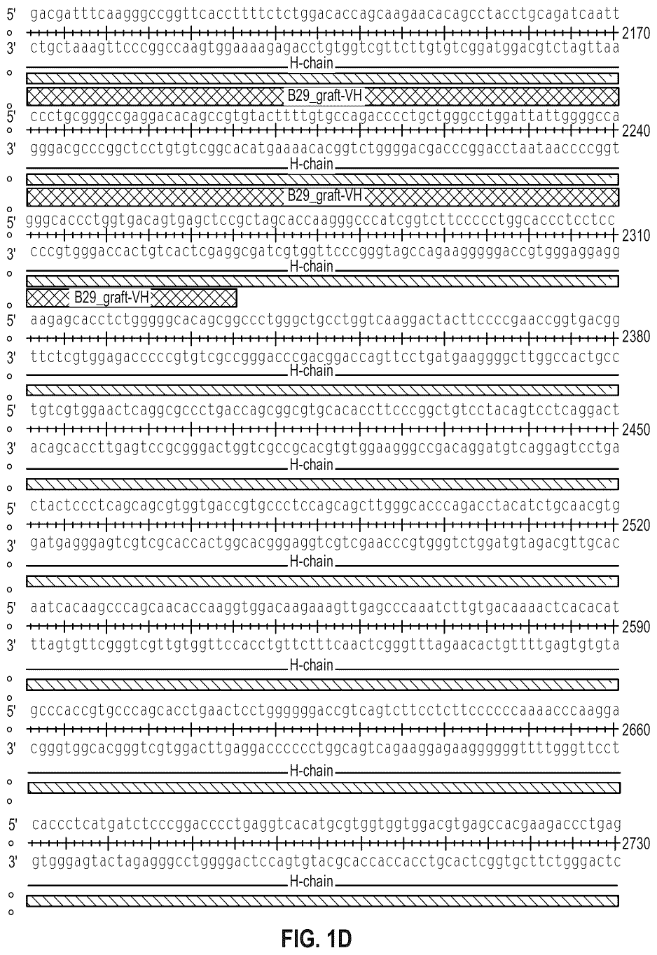

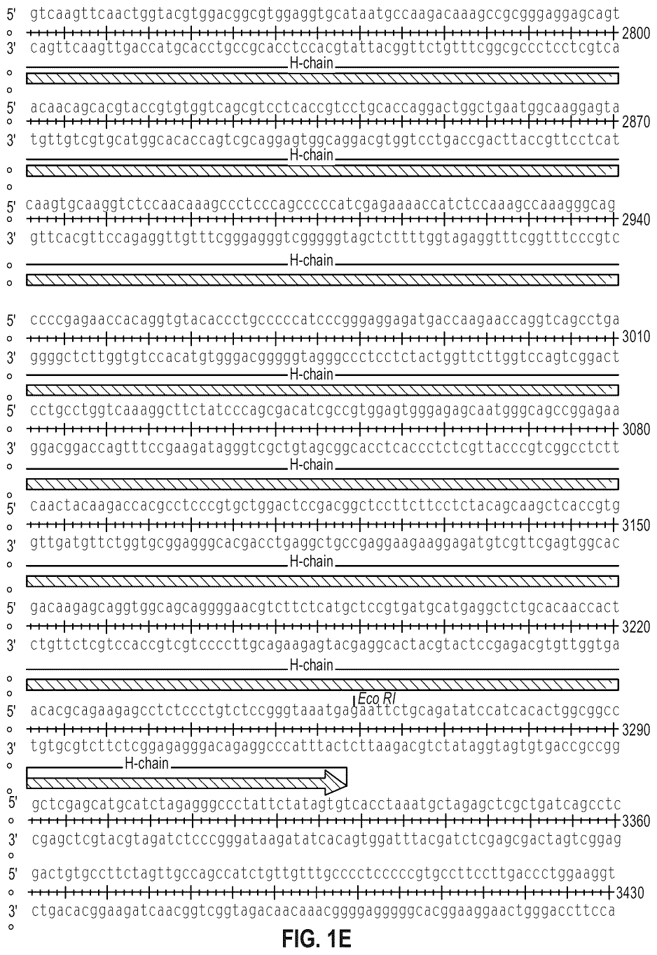

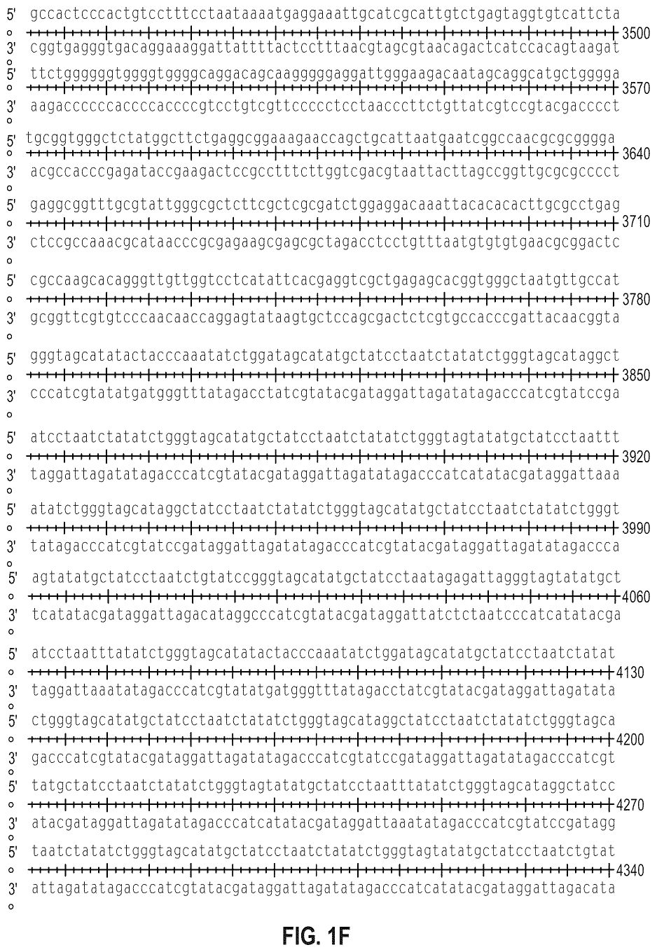

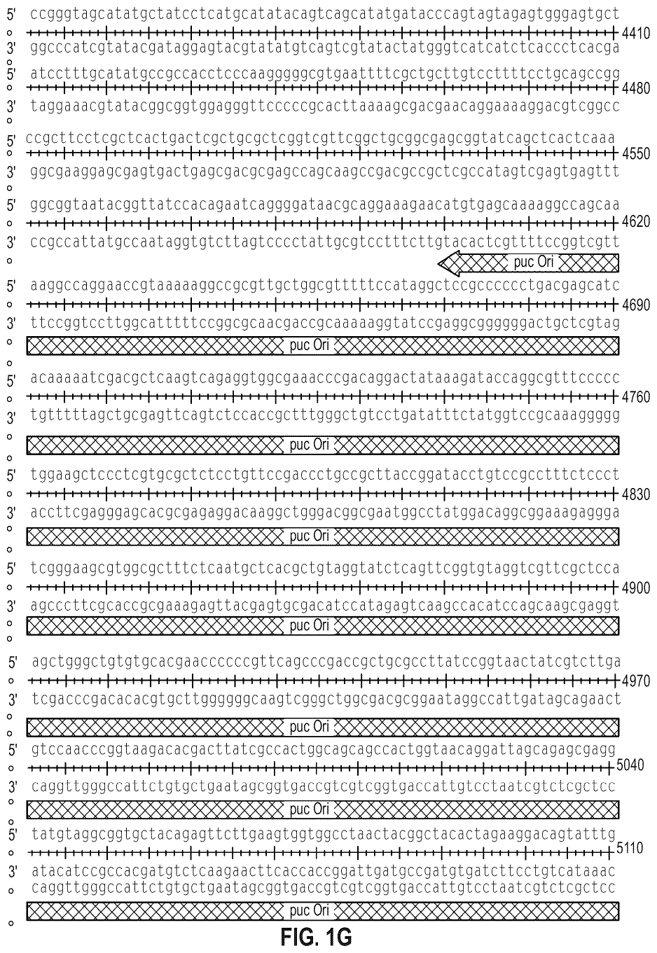

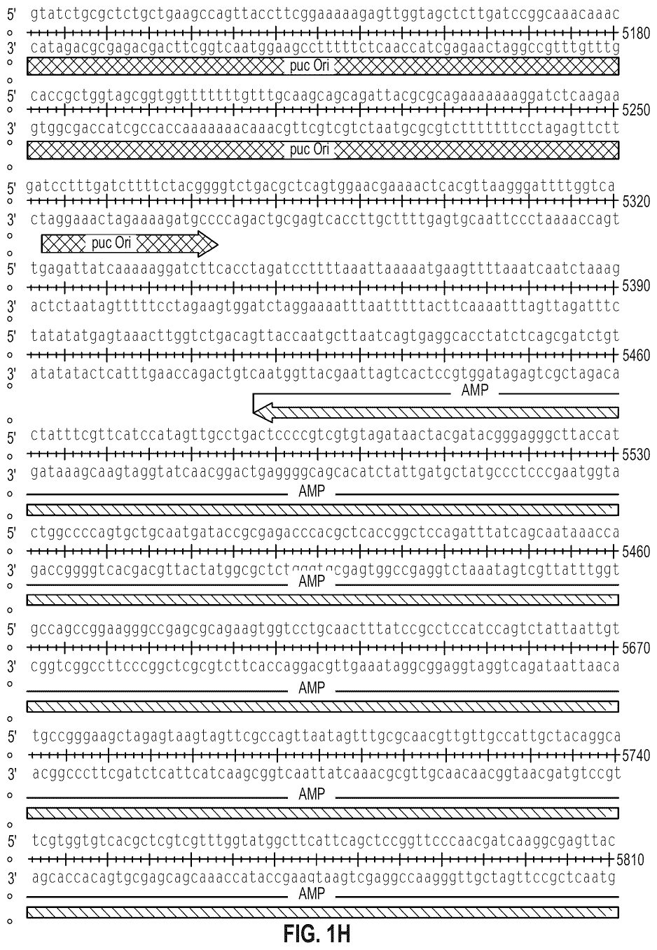

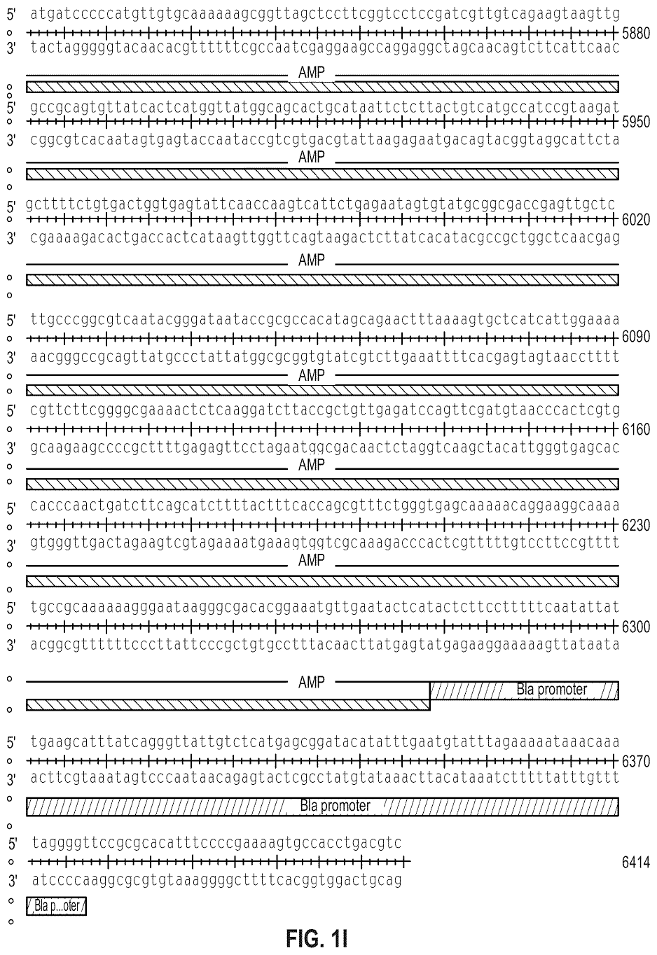

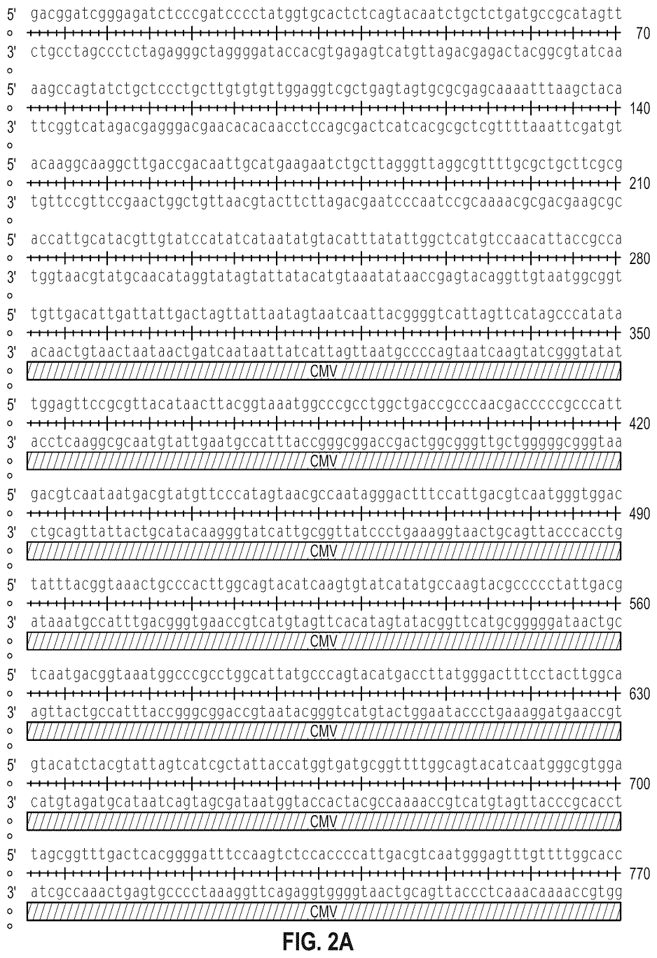

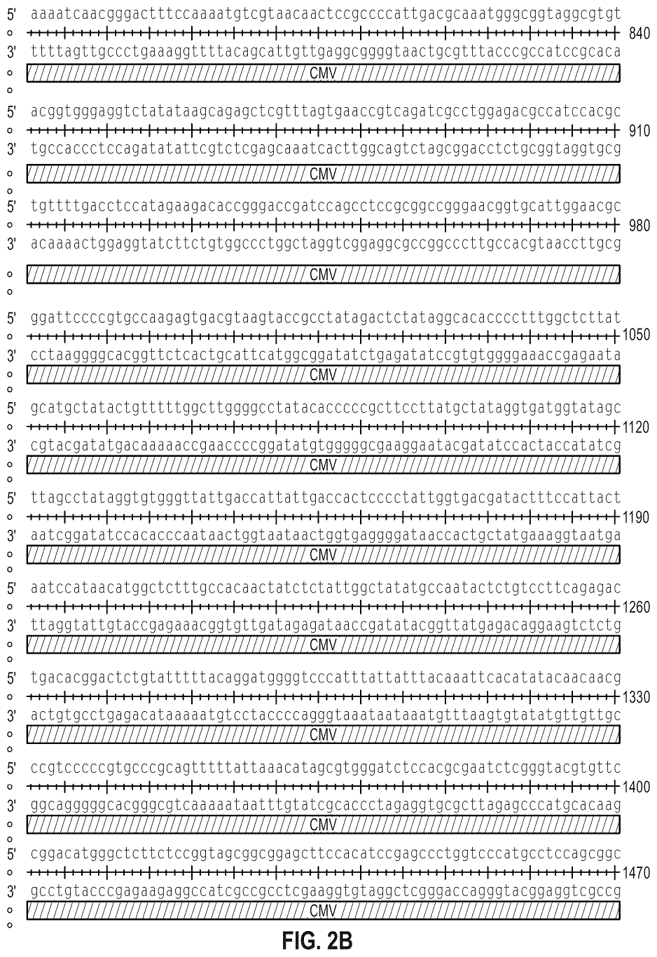

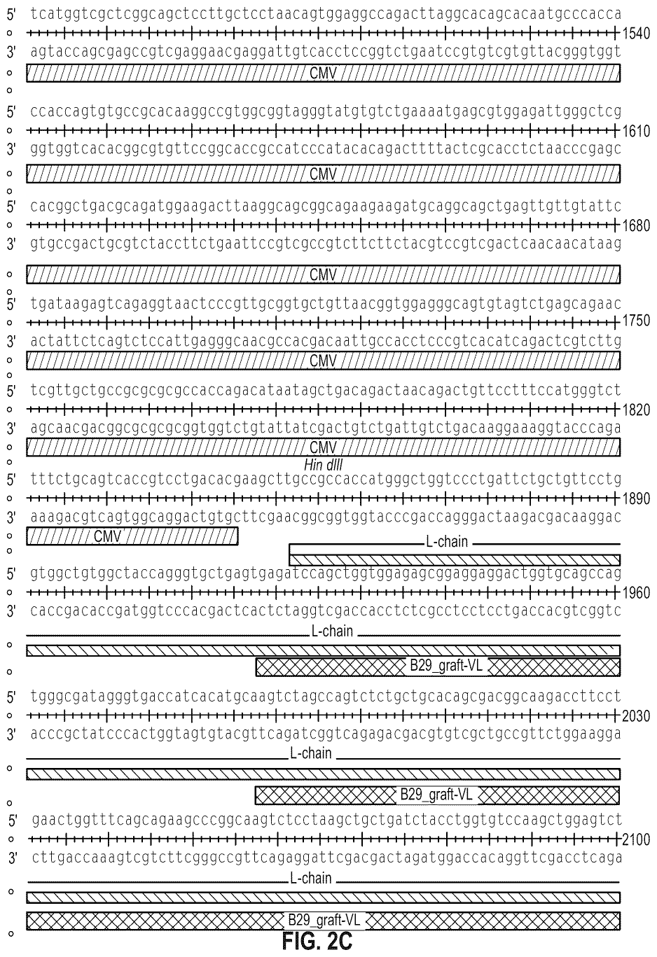

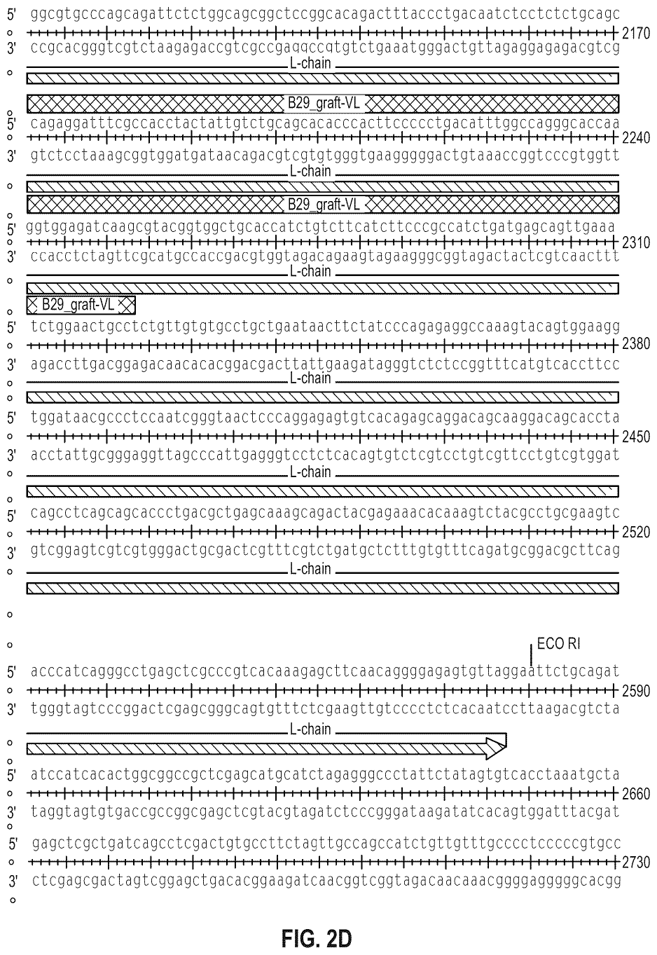

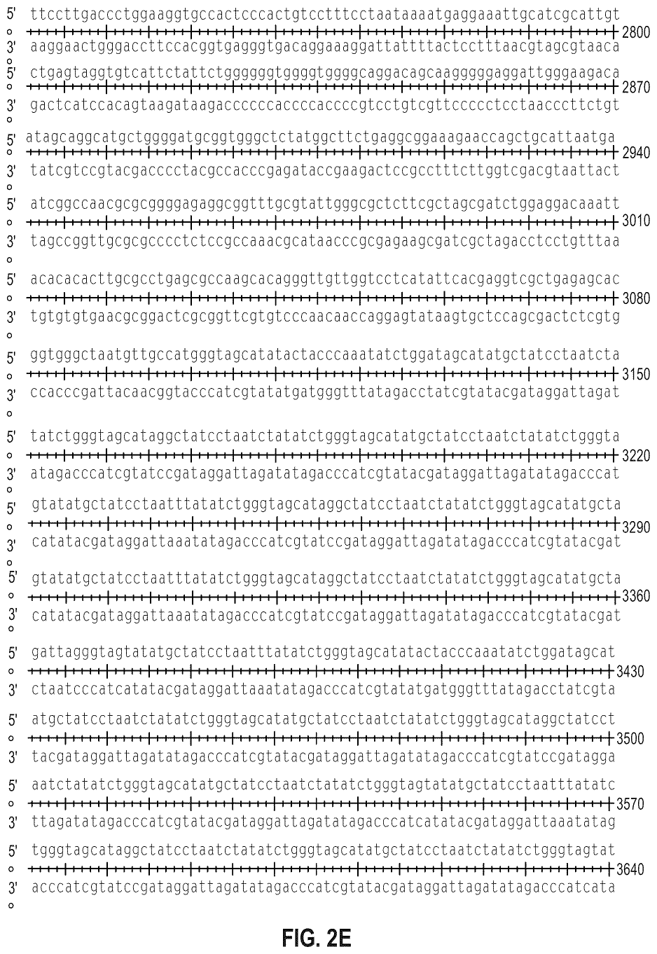

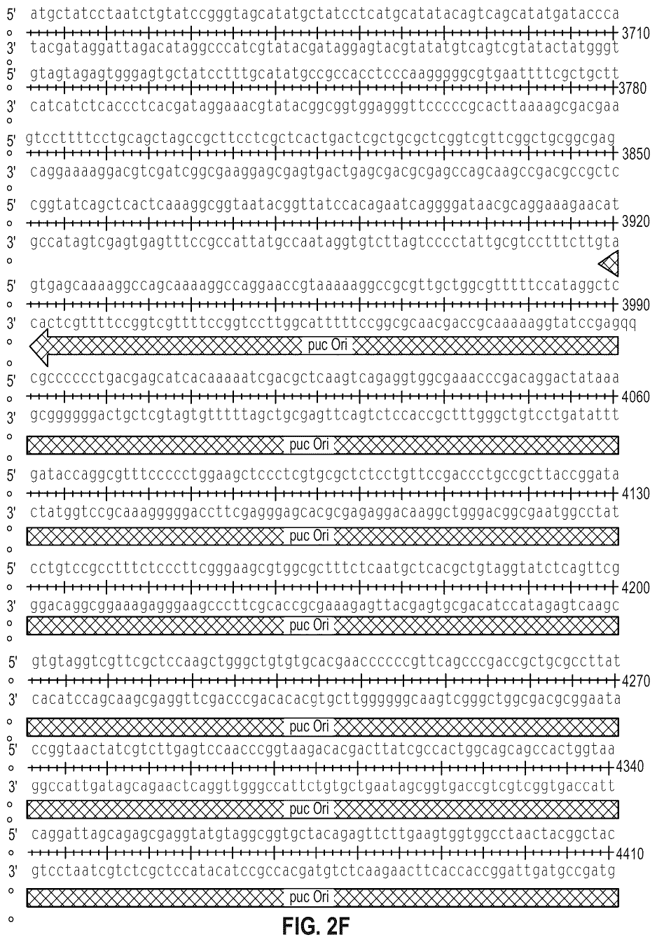

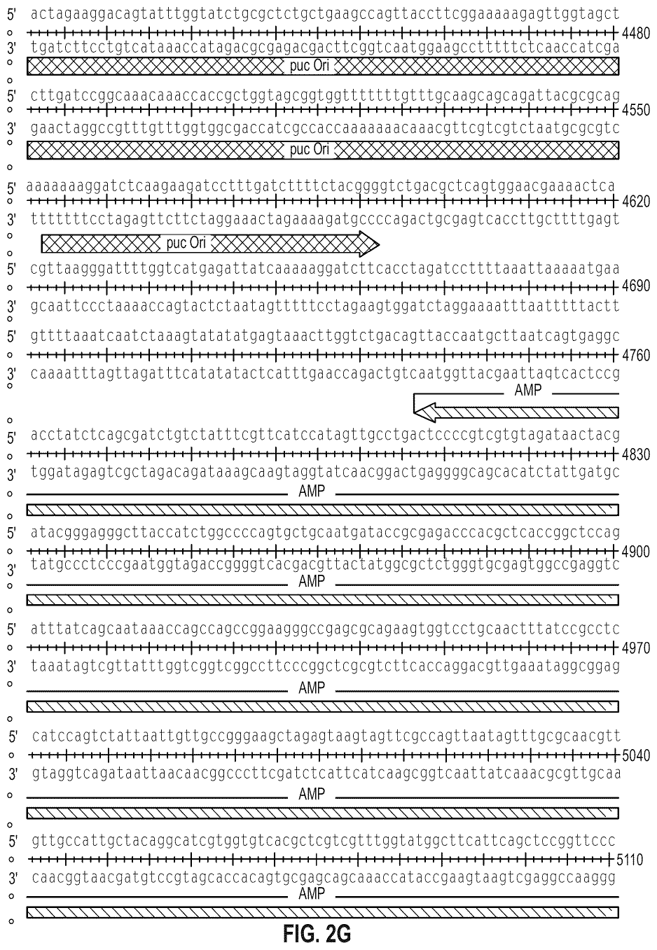

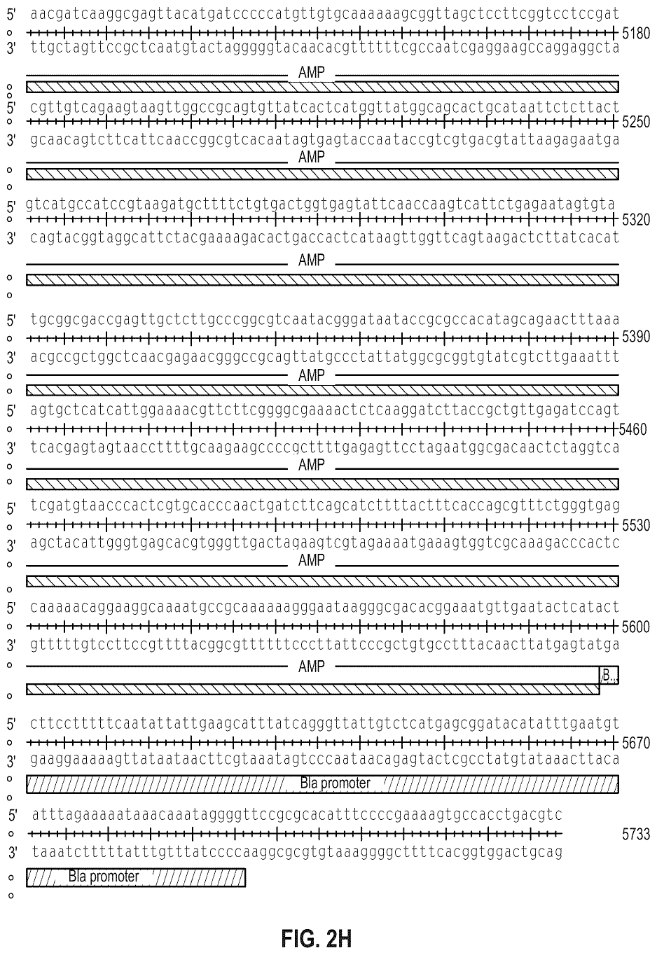

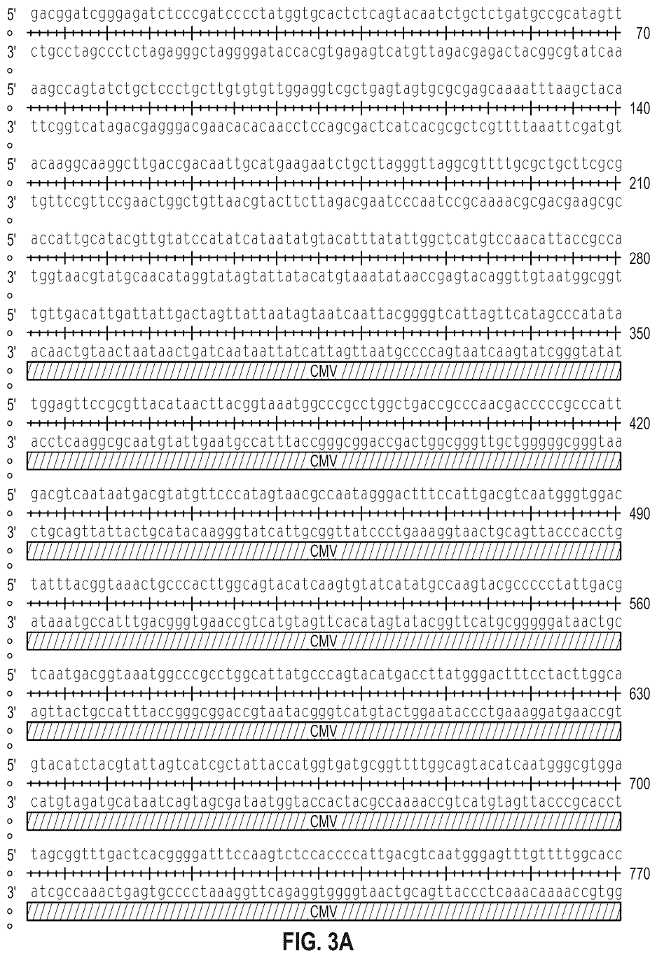

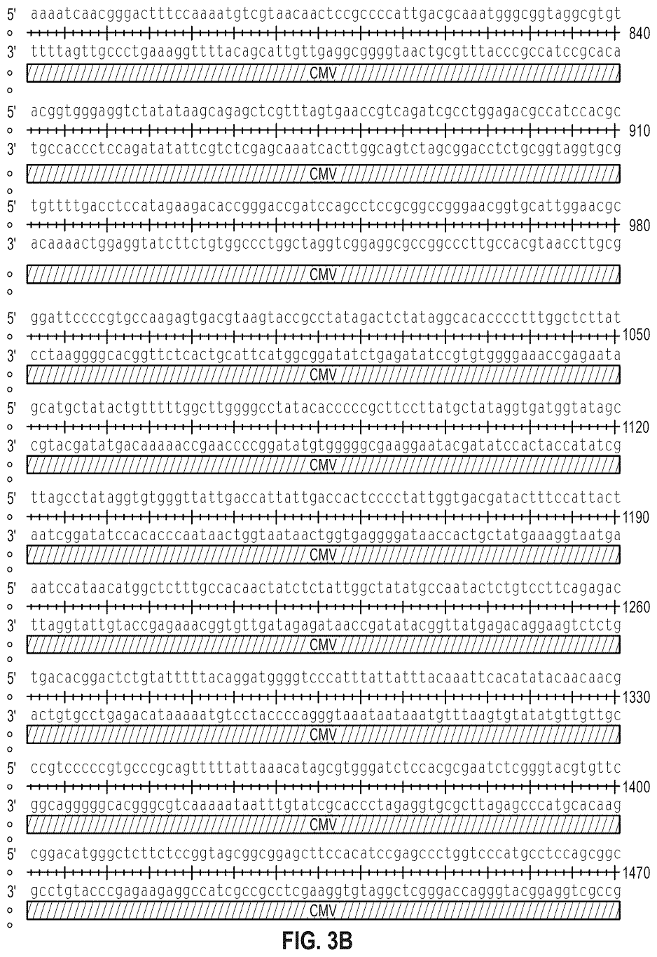

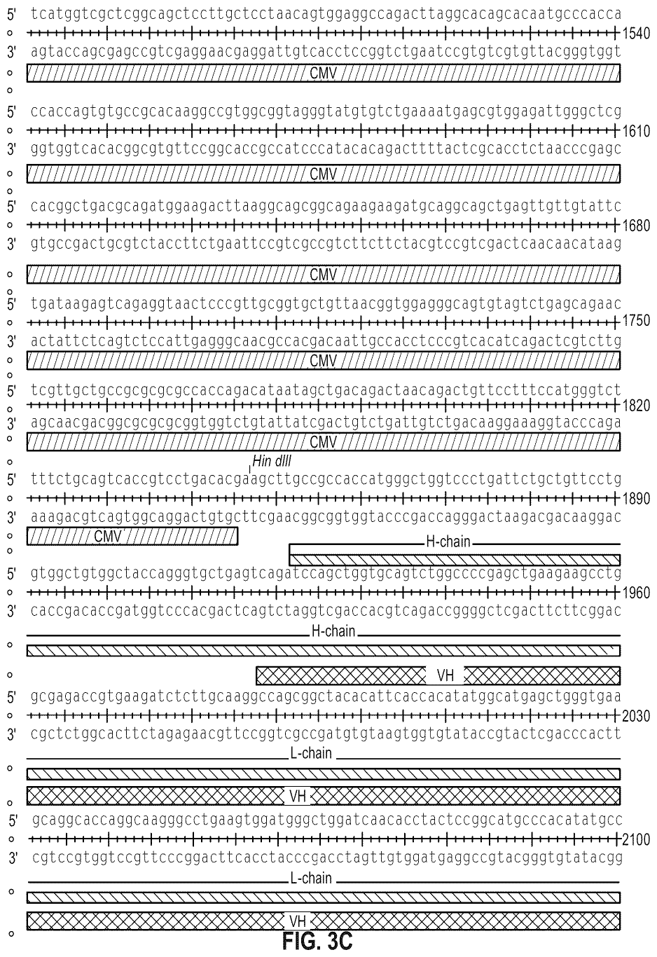

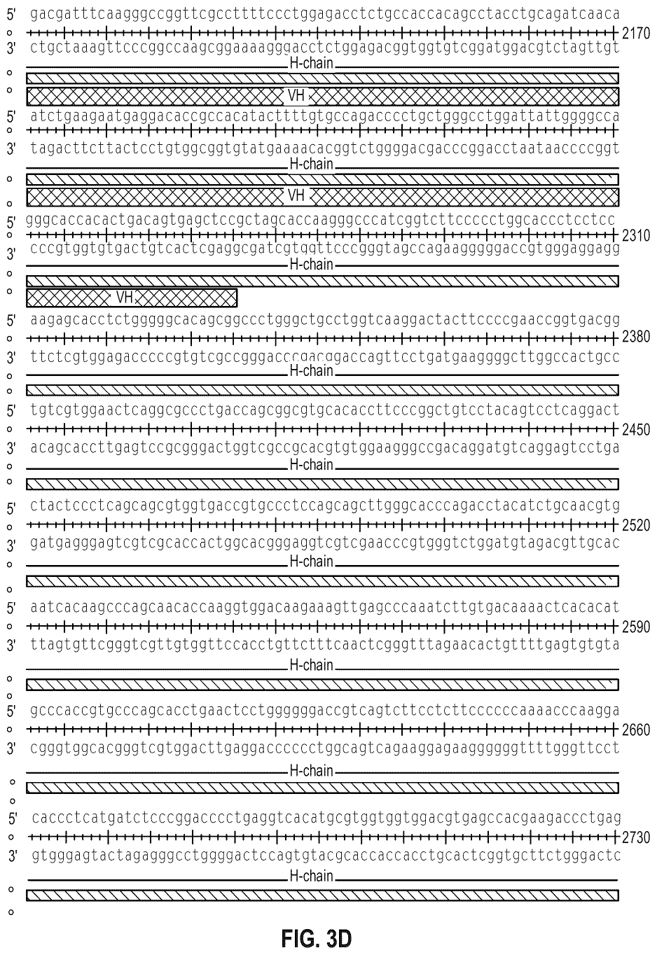

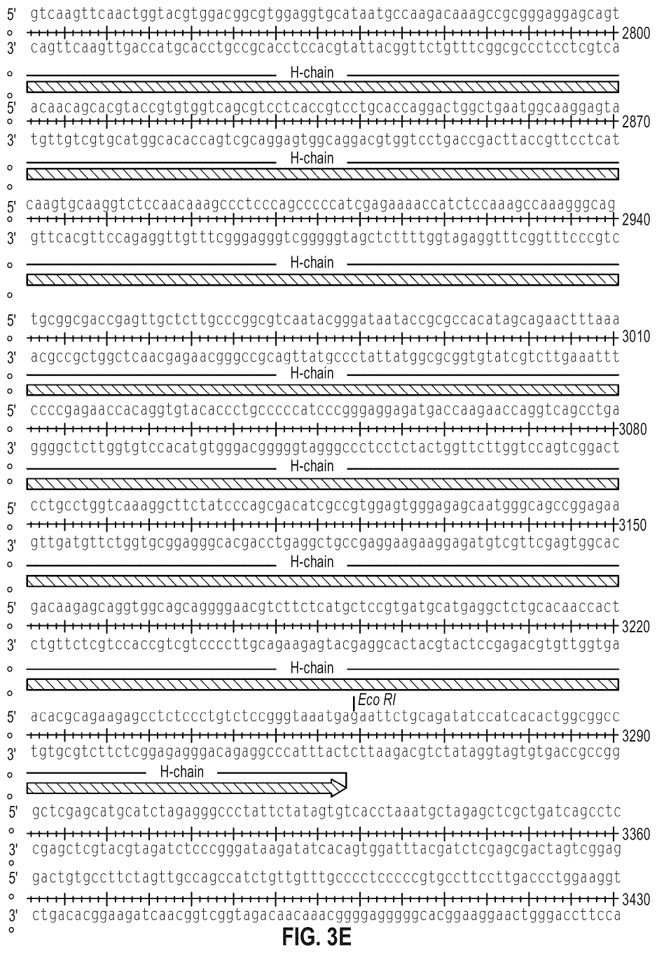

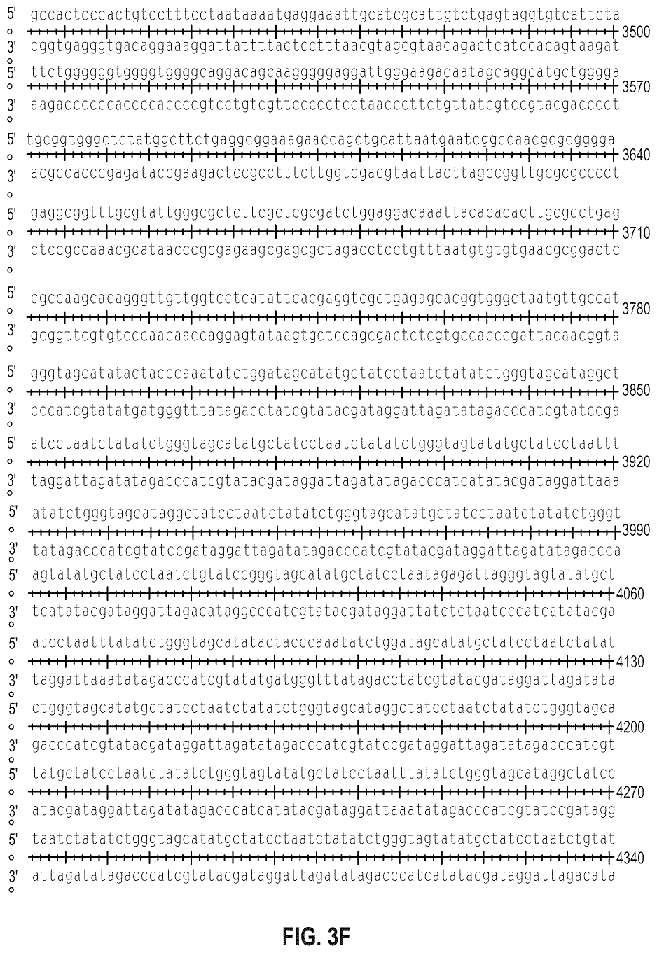

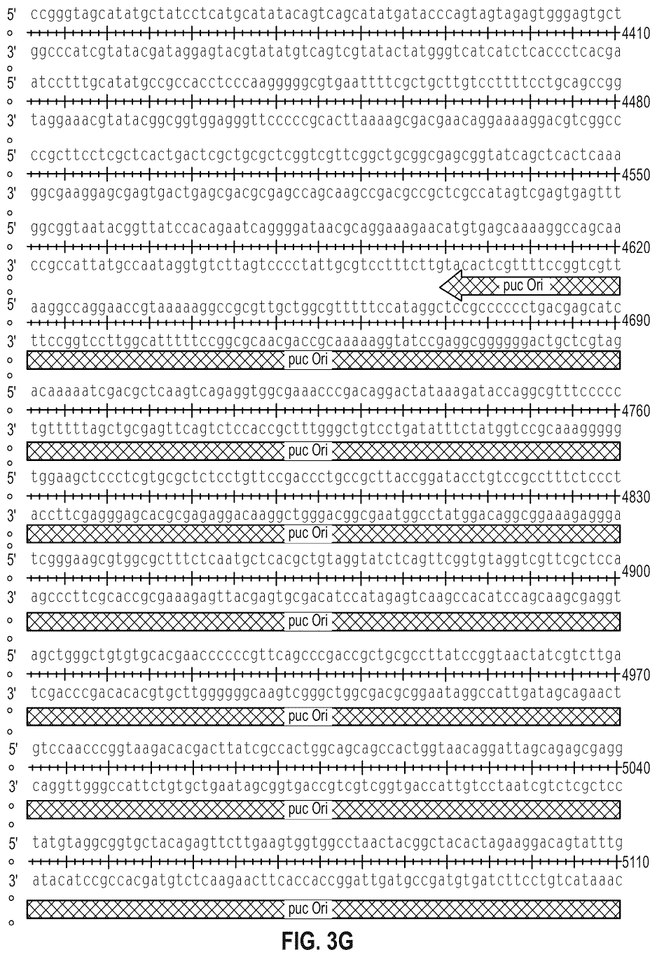

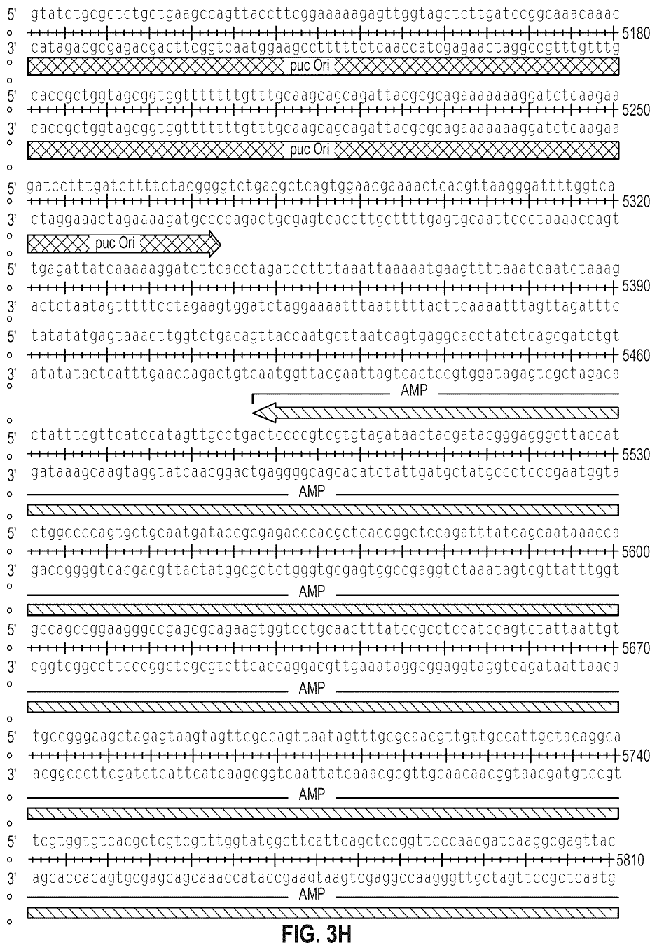

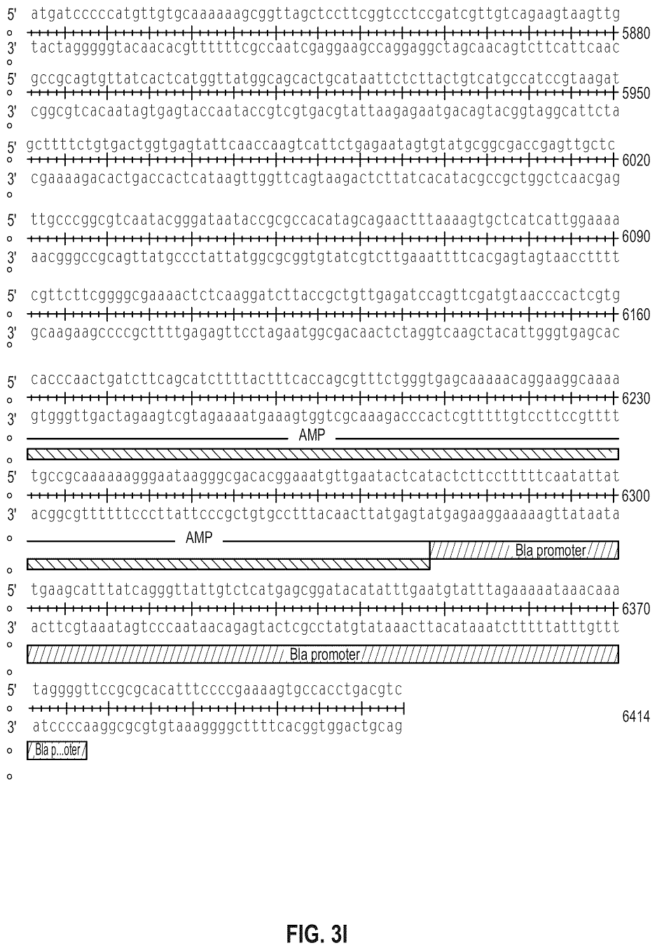

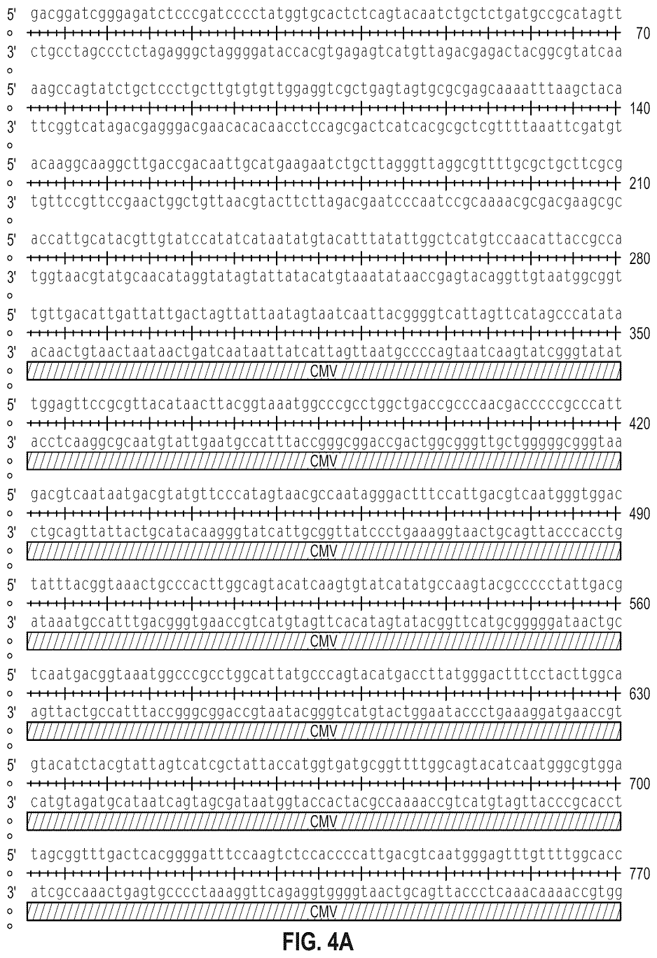

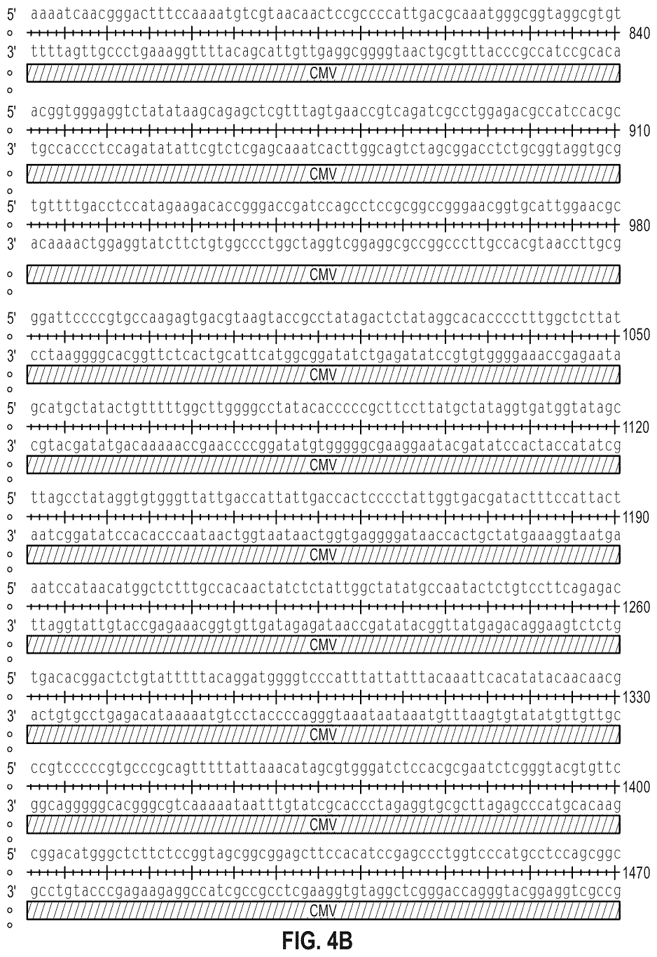

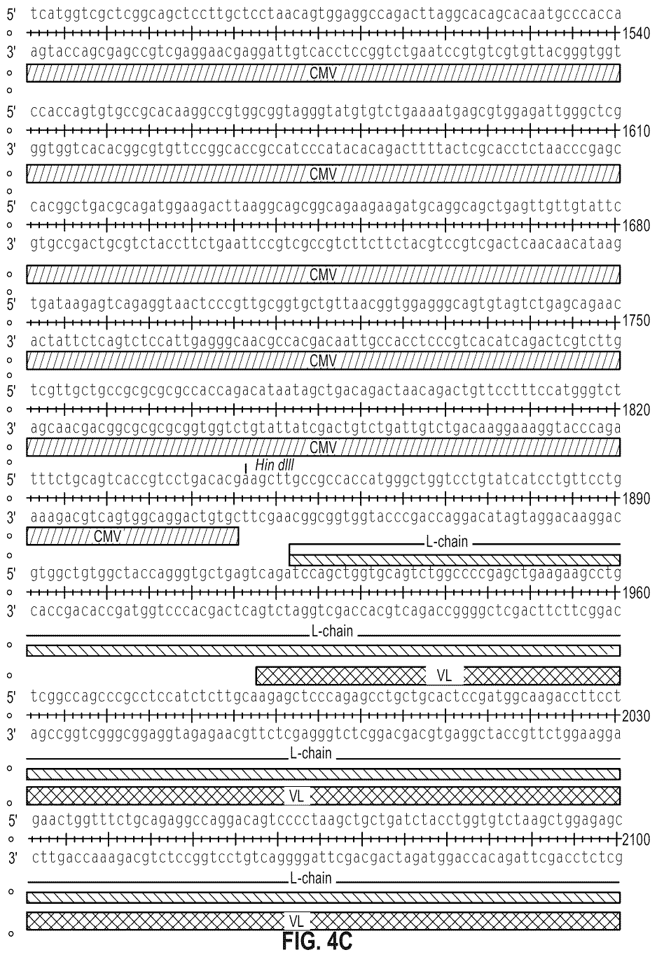

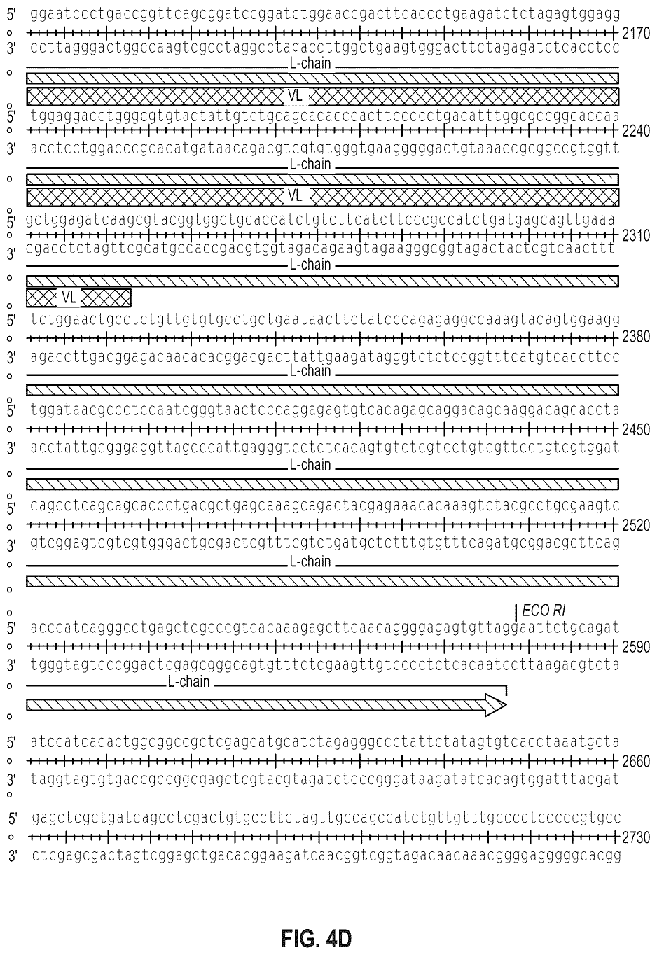

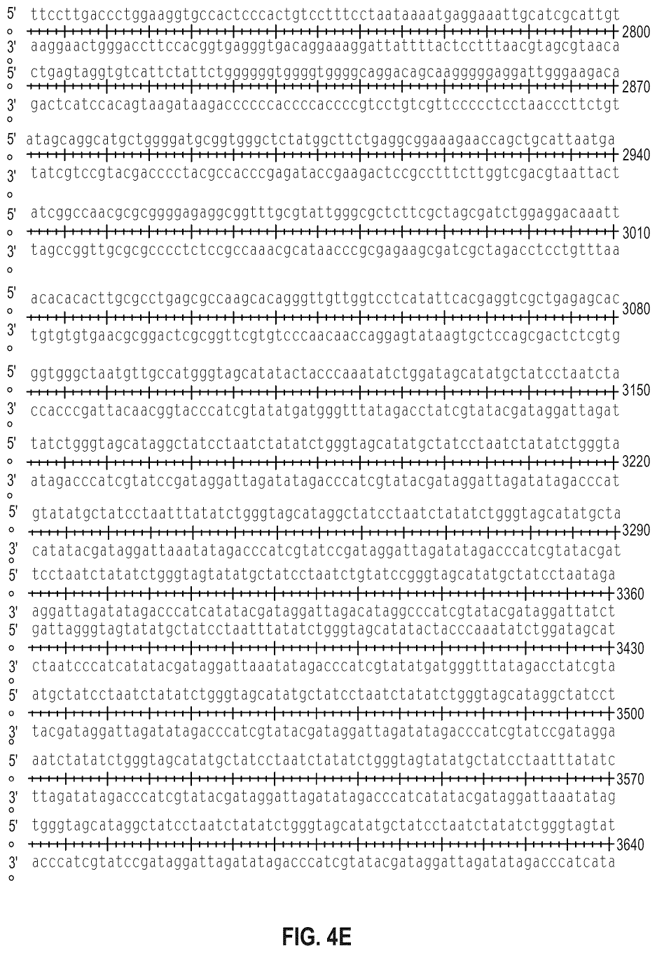

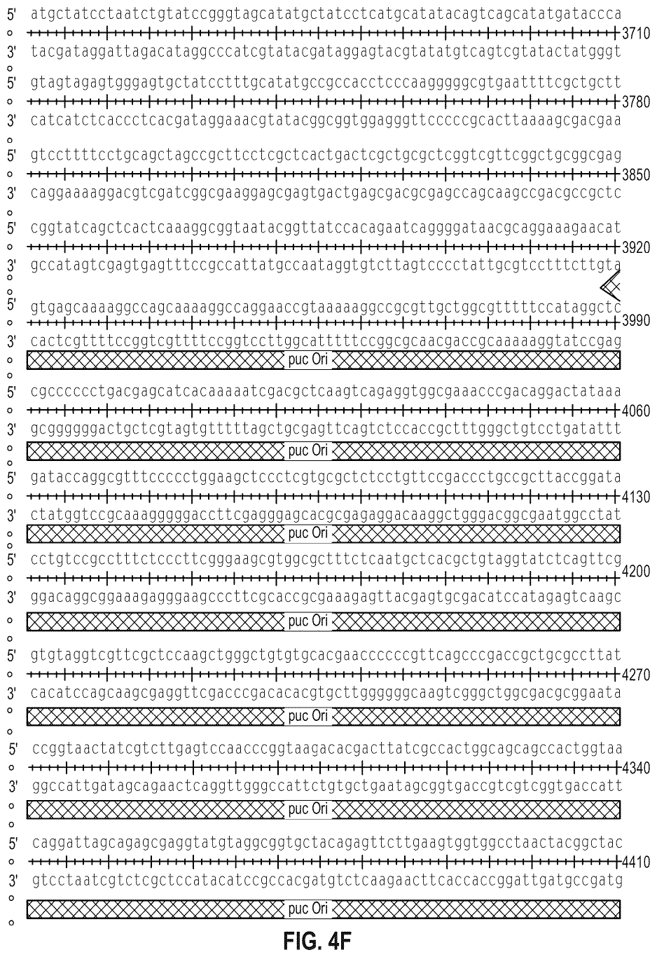

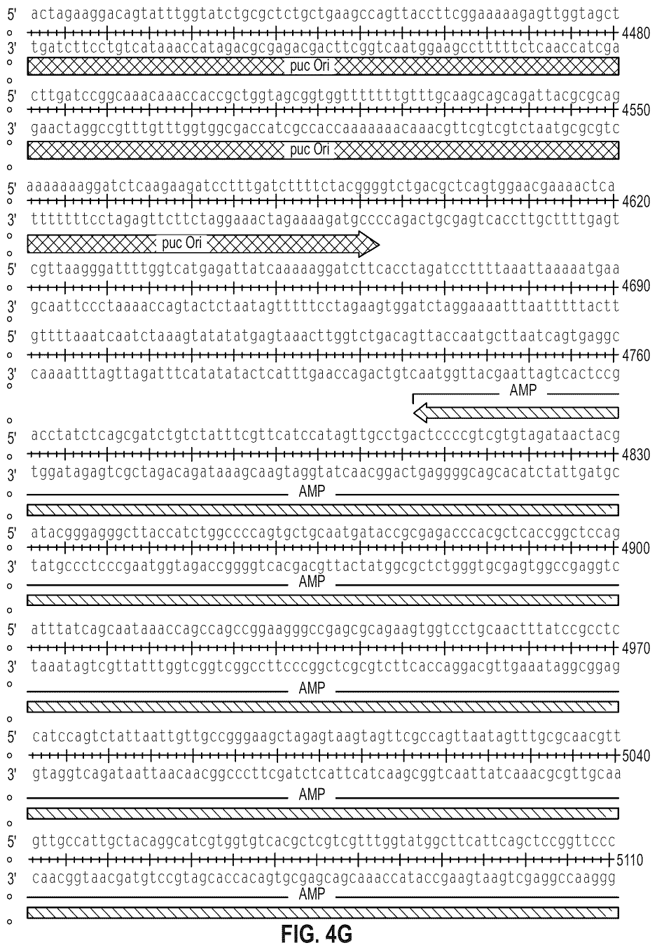

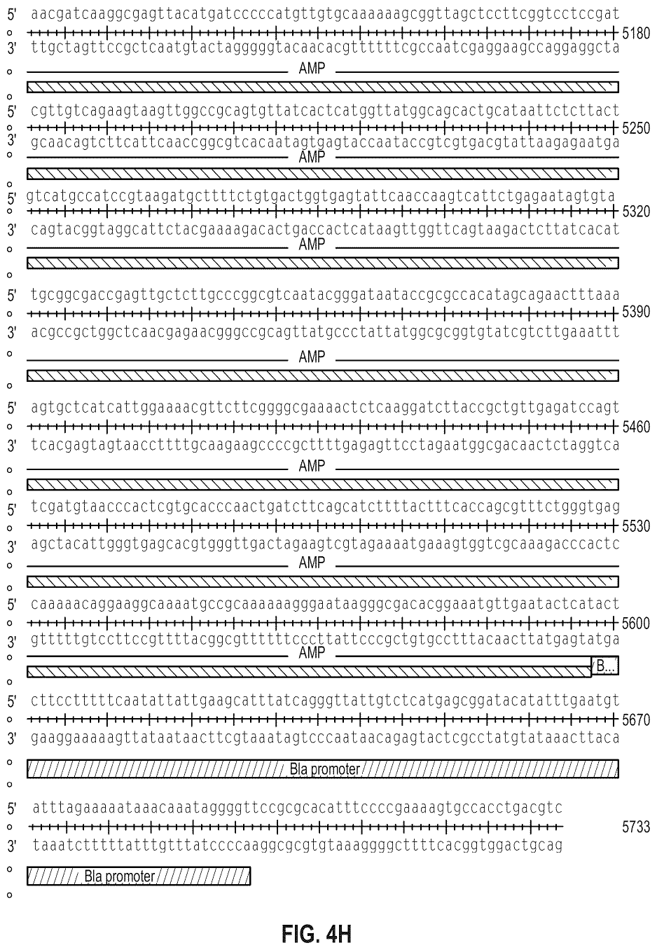

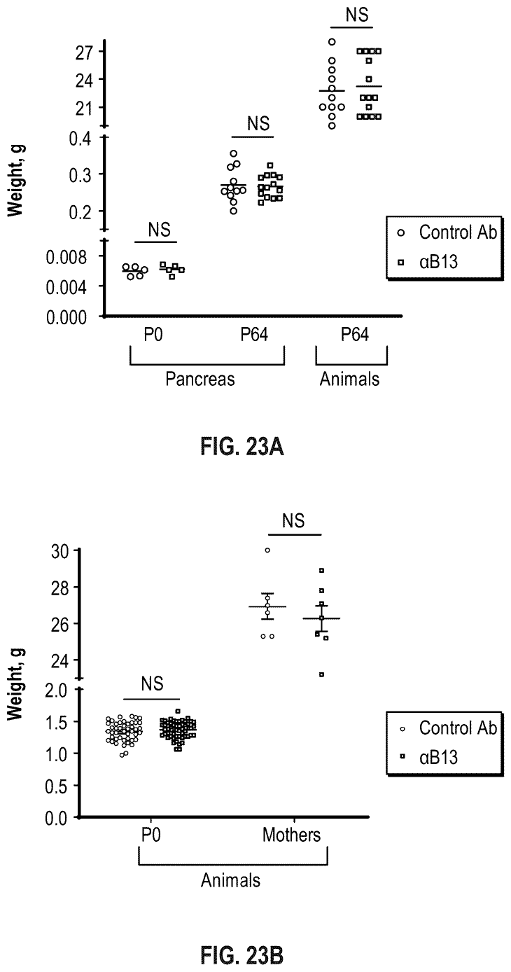

The nucleic acid sequence of B29 graft heavy chain is provided in FIGS. 1A-1I. The nucleic acid sequence of B29_graft light chain is provided in FIGS. 2A-2H. The nucleic acid sequence of B29_chimeric heavy chain is provided in FIGS. 3A-31. The nucleic acid sequence of B29_chimeric light chain is provided in FIGS. 4A-4H. In some embodiments, the isolated monoclonal antibodies or antigen-binding fragments thereof (1) bind to serpin B13, and (2) comprise a heavy chain CDR1, a heavy chain CDR2, and a heavy chain CDR3. The heavy chain CDR1 can comprise the amino acid sequence of SEQ ID NO: 1, 2 or 26, or the amino acid sequence of SEQ ID NO: 1, 2 or 26, with a substitution at one, two, or three amino acid positions. The heavy chain CDR2 can comprise the amino acid sequence of SEQ ID NO:4 or 27, or the amino acid sequence of SEQ ID NO:4 or 27 with a substitution at one, two, or three amino acid positions. The heavy chain CDR3 can comprise the amino acid sequence of SEQ ID NO: 6 or 28, or the amino acid sequence of SEQ ID NO:6 or 28 with a substitution at one, two, or three amino acid positions. In some embodiments, the isolated monoclonal antibodies or antigen-binding fragments can further include one or more of the following light chain CDRs: (1) a light chain CDR1 comprises the amino acid sequence of SEQ ID NO: 8 or 29, or the amino acid sequence of SEQ ID NO:8 or 29 with a substitution at one, two, or three amino acid positions; (2) a light chain CDR2 comprises the amino acid sequence of SEQ ID NO: 10, or the amino acid sequence of SEQ ID NO:10 with a substitution at one, two, or three amino acid positions; and (3) a light chain CDR3 comprises the amino acid sequence of SEQ ID NO:12, or the amino acid sequence of SEQ ID NO: 12 with a substitution at one, two, or three amino acid positions. In some embodiments, no amino acid substitutions are present in any of the above-described heavy chain CDR1, CDR2 or CDR3. In some embodiments, the one, two or three amino acid substitutions are made in positions other than those positions where a conserved amino acid residue is observed in the heavy chain CDR1, CDR2 or CDR3. In some embodiments, no amino acid substitutions are present in any of the above-described light chain CDR1, CDR2 or CDR3. In some embodiments, the one, two or three amino acid substitutions are made in positions other than those positions where a conserved amino acid residue is observed in the light chain CDR1, CDR2 or CDR3. In certain embodiments, the amino acid sequence of the protein is modified, for example by substitution, to create a polypeptide having substantially the same or improved qualities as compared to the original polypeptide. The substitution may be a conserved substitution. A “conserved substitution” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall peptide retains its spatial conformation but has altered biological activity. For example, common conserved changes might be Asp to Glu, Asn or Gln; His to Lys, Arg or Phe; Asn to Gln, Asp or Glu and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cystine, tyrosine, asparagine and glutamine having uncharged polar side chains; aspartate and glutamate having acidic side chains; and lysine, arginine, and histidine having basic side chains. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Conservative amino acid substitutions typically include substitutions within the same family. In some embodiments, the monoclonal antibodies and antigen-binding fragments disclosed herein comprise the heavy chain CDR1-3 described above and one or more of the light chain CDRs described herein.

One can use the hydropathic index of amino acids in conferring interactive biological function on a polypeptide, wherein it is found that certain amino acids may be substituted for other amino acids having similar hydropathic indices and still retain a similar biological activity. Alternatively, substitution of like amino acids may be made based on hydrophilicity, particularly where the biological function desired in the polypeptide to be generated in intended for use in immunological embodiments. The greatest local average hydrophilicity of a protein, as governed by the hydrophilicity of its adjacent amino acids, correlates with its immunogenicity. Accordingly, it is noted that substitutions can be made based on the hydrophilicity assigned to each amino acid. In using either the hydrophilicity index or hydropathic index, which assigns values to each amino acid, it is preferred to conduct substitutions of amino acids where these values are 2, with 1 being particularly preferred, and those with in 0.5 being the most preferred substitutions.

In certain embodiments, the one, two, or three amino acid substitutions are conservative amino acid substitutions.

### Chimeric and Humanized Antibodies

Recombinant forms of antibodies, such as chimeric and humanized antibodies, were prepared to minimize the response by a human patient to the antibody. When antibodies produced in non-human subjects or derived from the expression of non-human antibody genes are used therapeutically in humans, they are recognized to varying degrees as foreign, and an immune response may be generated in the patient. One approach to minimize or eliminate this immune reaction is to produce chimeric antibody derivatives, i.e., antibody molecules that combine a non-human animal variable region and a human constant region. Such antibodies retain the epitope binding specificity of the original monoclonal antibody, but may be less immunogenic when administered to humans, and therefore more likely to be tolerated by the patient. For example, one or all (e.g., one, two, or three) of the variable regions of the light chain(s) and/or one or all (e.g., one, two, or three) of the variable regions the heavy chain(s) of a mouse antibody (e.g., a mouse monoclonal antibody) can each be joined to a human constant region, such as, without limitation an IgG1 human constant region.

A chimeric antibody is further “humanized” by replacing portions of the variable region not involved in antigen binding with equivalent portions from human variable regions.

In the present invention, humanized antibodies were engineered to comprise one or more human framework regions in the variable region together with non-human (mouse) complementarity-determining regions (CDRs) of the heavy and/or light chain. In some embodiments, a humanized antibody comprises sequences that are entirely human except for the CDR regions. Humanized antibodies are typically less immunogenic to humans, relative to non-humanized antibodies, and thus offer therapeutic benefits in certain situations.

As used herein, “framework region” (FR) refers to amino acid sequences within the variable region of both heavy and light chain polypeptides that are not CDR sequences and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. In some embodiments, humanized versions of the monoclonal antibodies described herein can be made by replacing one or more (e.g., one, two, three, four, five, or six) framework regions of the antibodies described herein, with one or more (e.g., one, two, three, four, five, or six) human framework regions.

In certain embodiments, the monoclonal antibodies and antigen-binding fragments disclosed herein comprises the light chain CDR1, CDR2 or CDR3 described above and one or more of the heavy chain CDRs described herein.

In certain embodiments, the monoclonal antibodies and antigen-binding fragments disclosed herein (1) bind to serpin B13, and (2) comprise a heavy chain variable region that is at least 65%, e.g., 65%, 70%, 75%, 80%, 85%, 90%, 95%, 100%, identical to SEQ ID NO: 18, and a light chain variable region that is at least 65%, e.g., 65%, 70%, 75%, 80%, 85%, 90%, 95%, 100%, identical to SEQ ID NO:20.

In certain embodiments, the monoclonal antibodies and antigen-binding fragments disclosed herein (1) bind to serpin B13, and (2) comprise a heavy chain variable region that is at least 65%, e.g., 65%, 70%, 75%, 80%, 85%, 90%, 95%, 100%, identical to SEQ ID NO:22, and a light chain variable region that is at least 65%, e.g., 65%, 70%, 75%, 80%, 85%, 90%, 95%, 100%, identical to SEQ ID NO:24.

In some embodiments, the monoclonal antibodies and antigen-binding fragments disclosed herein bind to serpinB13 with an affinity of about 1 nM to about 8 nM. In certain embodiments, the monoclonal antibodies and antigen-binding fragments disclosed herein bind to serpin B13 with an affinity of about 1 nM to about 2 nM (e.g., 1.21 nM).

### Methods of Using the Monoclonal Antibodies and Antibody Fragments

The antibodies and antigen-binding fragments described herein are used to inhibit or reduce serpin B13 and treat serpin B13-related disorders, e.g., type 1 diabetes, Methods of treating a serpin B13-related disorders in a subject can include (a) identifying a subject having an serpin B13-related disorders; and (b) administering to the subject an effective amount of one or more different ones of the monoclonal antibodies disclosed herein. In some embodiments, the subject is a human.

The serpin B13-related disorder can be, for example, diabetes, such as type I diabetes, type 2 diabetes, and diabetes in patients with chronic pancreatitis who undergo total pancreatectomy with autologous islet transplantation and still remain insulin dependent. In some embodiments, the new monoclonal antibodies disclosed herein are used to treat a serpin B13-related disorder, wherein the disorder is inflammatory or central nervous system disease. In some embodiments, the new monoclonal antibodies disclosed herein are used to treat bone fracture, skin wound/ulcer healing including diabetic foot, hair loss, multiple sclerosis, or lupus.

### Formulations and Methods of Administration

The compositions of the invention may be formulated as pharmaceutical compositions (e.g., comprising fusion proteins or expression vectors) and administered to a mammalian host, such as a human patient, in a variety of forms adapted to the chosen route of administration, i.e., orally, intranasally, intradermally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard- or soft-shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with several of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

The new monoclonal antibodies disclosed herein, or antigen-binding fragments thereof, can be administered in an effective amount, at dosages and for periods of time necessary to achieve the desired result. An “effective amount” is an amount sufficient to effect beneficial or desired results. For example, a therapeutically effective amount is one that achieves the desired therapeutic effect. An effective amount can be administered in one or more administrations, applications or dosages. A therapeutically effective amount of a pharmaceutical composition (i.e., an effective dosage) depends on the pharmaceutical composition selected. The compositions can be administered from one or more times per day to one or more times per week, including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the pharmaceutical compositions described herein can include a single treatment or a series of treatments.

Dosage regimens can be adjusted to provide the optimum therapeutic response. For example, several divided doses can be administered daily, or the dose can be proportionally reduced as indicated by the exigencies of the therapeutic situation. Those skilled in the art will be aware of dosages and dosing regimens suitable for administration of the new monoclonal antibodies disclosed herein or antigen-binding fragments thereof to a subject. See e.g., Physicians' Desk Reference, 63rd edition, Thomson Reuters, Nov. 30, 2008. For example, Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, the monoclonal antibodies described herein are administered intravenously at about 0.1-20 mg/kg, e.g., about 0.5-15 mg/kg, about 1-12 mg/kg, about 2-10 mg/kg.

### Kits

Also provided herein are kits that include at least one (e.g., two, three, four, five, or more) compositions containing at least one (e.g., one, two, three, four, five, or more different ones) of the isolated new monoclonal antibodies or antigen-binding fragments thereof described herein. In some embodiments, the kits described herein contain one or more humanized or human version of the monoclonal antibodies or antigen-binding fragments thereof.

Kits generally include the following major elements: packaging, reagents comprising binding compositions as described above, optionally a control, and instructions. Packaging can be a box-like structure for holding a vial (or number of vials) containing said binding compositions, a vial (or number of vials) containing a control, and instructions for use in a method described herein. Individuals skilled in the art can readily modify the packaging to suit individual needs.

In some embodiments, a kit provided herein can include at least one (e.g., one, two, three, four, five, or more) composition containing at least one (e.g., one, two, three, four, five, or more) of the isolated new monoclonal antibodies or antigen-binding fragments thereof described herein.

Compositions and kits as provided herein can be used in accordance with any of the methods (e.g., treatment methods) described above. For example, compositions and kits containing at least one (e.g., one, two, three, four, five, or more) of the isolated new monoclonal antibodies or antigen-binding fragments thereof described herein can be used to treat serpin B13-related disorder, e.g., type I diabetes. Those skilled in the art will be aware of other suitable uses for compositions and kits provided herein and will be able to employ the compositions and kits for such uses.

The invention will now be illustrated by the following non-limiting Examples.

### Example 1

### Inhibition of SerpinB13 Stimulates Beta-Cell Development Via Notch Signaling Pathway

Methods for repopulating the pancreas with new insulin-producing cells have strong potential for therapy in diabetes. Recently, it was found that inhibition of serpinB13, which is a protease inhibitor of cathepsin L (catL), with mAb in mouse embryos lead to a robust increase in the number of pancreatic Ngn3⁺ progenitor cells, significant expansion of islet mass, and improved resistance to severe diabetes in adulthood.

To unveil the molecular mechanism of the augmented Ngn3⁺ cell response following inhibition of serpinB13 during gestation, the Notch communication system (a critical signaling pathway for pancreatic development) was studied. It was found that serpinB13 is expressed and secreted by epithelial cells in murine embryonic pancreases. Moreover, in vivo and in vitro inhibition of serpinB13 during embryogenesis caused protease-dependent cleavage of the extracellular domain of Notch1 receptor in the pancreas (p<0.0001). This partial loss of the extracellular Notch was followed by decreased translocation to the nucleus of active Notch intracellular domain (aNICD), a fragment of Notch that is critical for restraining endocrine cell development. Finally, embryonic pancreases of mice with genetic deficiency of catL had significantly fewer Ngn3⁺ cells compared with wild type controls.

Together, the data point to a novel function of serpinB13 in maintaining Notch receptor-mediated repression of pancreatic endocrine progenitors. Consequently, the perturbation of this effect of serpinB13 enables protease activity to partially dismantle Notch signaling, thereby allowing for more efficient development of Ngn3⁺ progenitors cells and a subsequent increase in islet mass.

### Example 2

Cell lysates in TriZol solution were provided and cDNA was obtained from the total RNA followed by PCR amplification of the variable regions (both heavy and light chains) of the antibody. The resulted PCR fragments were then cloned into a standard vector separately and sequenced. Based on the gel analysis of PCR products, the type of hybridoma B29 light chain is kappa. The sequence information is the following:

B29 Heavy Chain
Amino acid sequence (CDR region in bold)
FR1-CDR1-FR2-CDR2-FR3-CDR3-FR4
(SEQ ID NO: 14)
QIQLVQSGPELKKPGETVKISCKASGYTFTTYGMSWVKQAPGKGLKWMGWINTYSGMPTYADDF
KGRFAFSLETSATTAYLQINNLKNEDTATYFCARPLLGLDYWGQGTTLTVSS
Nucleotide sequence (CDR region in bold)
FR1-CDR1-FR2-CDR2-FR3-CDR3-FR4
(SEQ ID NO: 15)
CAGATCCAGTTGGTACAGTCTGGACCTGAGCTGAAGAAGCCTGGAGAGACAGTCAAGATCTCCT
GCAAGGCCTCCGGGTATACCTTCACAACCTATGGAATGAGCTGGGTGAAACAGGCTCCAGGAAA
GGGTTTAAAGTGGATGGGCTGGATAAACACCTACTCTGGAATGCCAACATATGCTGATGACTTC
AAGGGACGGTTTGCCTTCTCTTTGGAAACCTCTGCCACCACTGCCTATTTGCAGATCAACAACC
TCAAAAATGAGGACACGGCTACATATTTCTGTGCAAGACCTCTCCTGGGACTTGACTATTGGGG
CCAAGGCACCACTCTCACAGTCTCCTCA
B29 Light Chain
Amino acid sequence (CDR region in bold)
FR1-CDR1-FR2-CDR2-FR3-CDR3-FR4
(SEQ ID NO: 16)
DIVMTQTPLSLSVTIGQPASISCKSSQSLLHSDGKTFLNWFLQRPGQSPKLLIYLVSKLESGIP
DRFSGSGSGTDFTLKISRVEVEDLGVYYCLQHTHFPLTFGAGTKLEIK
Nucleotide sequence (CDR region in bold)
FR1-CDR1-FR2-CDR2-FR3-CDR3-FR4
(SEQ ID NO: 17)
GATATTGTGATGACCCAGACTCCACTGTCTTTGTCGGTTACCATTGGACAACCAGCCTCCATCT
CTTGCAAGTCAAGTCAGAGCCTCTTACATAGTGATGGAAAGACATTTTTGAATTGGTTTTTACA
GAGGCCAGGCCAGTCTCCAAAGCTCCTAATCTATCTGGTGTCTAAACTGGAATCTGGCATCCCT
GACAGGTTCAGTGGCAGTGGATCAGGGACAGATTTCACACTGAAAATCAGCAGAGTGGAGGTTG
AGGATTTGGGAGTTTATTACTGCTTGCAACATACACATTTTCCGCTCACGTTCGGTGCTGGGAC
CAAACTGGAAATAAAA

After the B29 antibody sequence was obtained, comprehensive bioinformatics analysis of it was performed. It was determined that the heavy chain of B29 (B29_H) belongs to mouse IGHV9 subgroup and its nearest germline gene sequence is mouse IGHV9-3*01. The light chain (B29_L) belongs to mouse IGKV1 subgroup and its nearest germline gene sequence is mouse IGKV1-133*01. Then the risk evaluation of post-translation modification (PTM) sites in B29 CDR regions was performed. The B29_L CDRs had an asp isomerization risk site, aspartic acid0glicine (DG), while B29_H CDRs had three oxidation risk sites, two methionine (M) and on tryptophan and the overall developability risk of B29 antibody is low.

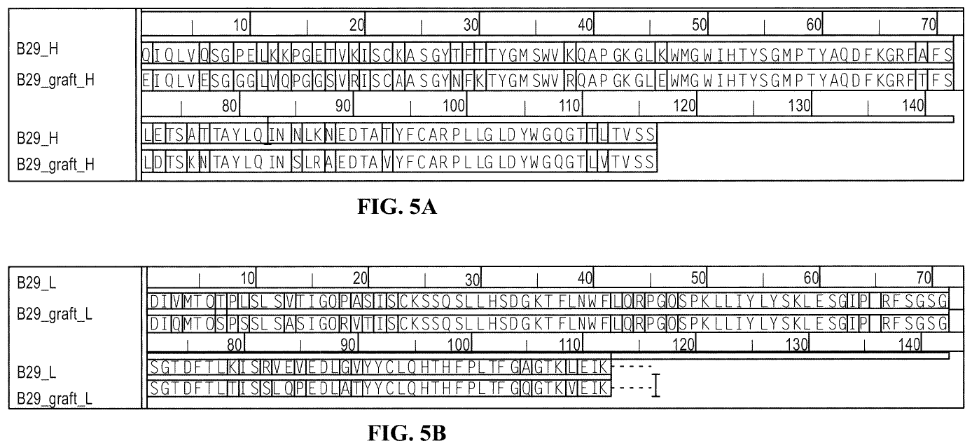

Based on the bioinformatics analysis result, human germline IGKV1_39*01 was chosen to perform the humanization of B29 light chain (B29_L) and human germline IGKV3_66*01 was used to humanize B29 heavy chain (B29_H). The sequence after CDR-grafting was named B29_graft_H. The sequence alignment of the germline gene before and after granting is shown in FIG. 5A. The antibody sequence after CDR-grafted is named B29_graft_L. The sequence alignment of the germline gene before and after grafting is shown in FIG. 5B. After CDR-grafting, the humanized heavy chain (named B29_graft_H) and light chain (named B29_graft_L) were obtained. The amino acid sequence of B29_graft is provided below:

B29_graft heavy chain
(SEQ ID NO: 18)
EIQLVESGGGLVQPGGSVRLSCAASGYNFKTYGMSWVRQAPGKGLEWMGWI
NTYSGMPTYADDFKGRFTFSLDTSKNTAYLQINSLRAEDTAVYFCARPLLG
LDYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHK
PSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSK
LTVDKSRWQQGNVFSCSVMHEALHNHYTQKSLSLSPGK
B29_graft light chain
(SEQ ID NO: 20)
DIQMTQSPSSLSASVGDRVTITCKSSQSLLHSDGKTFLNWFQQKPGKSPKL
LIYLVSKLESGVPSRFSGSGSGTDFTLTISSLQPEDFATYYCLQHTHFPLT
FGQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGEC

When the B29 graft antibody was evaluated, it was observed that both heavy chain and light chain had higher degree of humanization after CDR grafting. In detail, the B29_graft_L shares 94% identities with the human germline gene (CDR region excluded) and the B29_graft_H shares 86.6% identities with the human germline gene (CDR region excluded). And about the aggregation tendency prediction, the CDR regions of B29 has high aggregation tendency, and after humanization, the aggregation tendency is reduced.

The humanized antibody and mouse/human chimeric IgG were expressed in HEK293 cells. The expression yield of the humanized antibody was 100 mg/L, while that of the chimeric IgG was 10 mg/L. SDS-PAGE QC result showed that both the humanized and the chimeric antibody were in good quality.

ELISA and SPR assays were performed to verify the affinity of the humanized and chimeric antibody. Both of the two antibodies showed obvious binding affinity for the target antigen. Thermostability (Tm and Tagg values) of the antigen, humanized antibody and chimeric antibody was measured using UNcle system. The result showed that both Tm and Tagg values were enhanced after humanization.

### Amino Acid Sequences of the Humanized and Chimeric Antibodies

Amino acid sequences of the humanized and chimeric antibodies
B29_graft heavy chain
(SEQ ID NO: 18)
EIQLVESGGGLVQPGGSVRLSCAASGYNFKTYGMSWVRQAPGKGLEWMGWINTYSGMPTYADDF
KGRFTFSLDTSKNTAYLQINSLRAEDTAVYFCARPLLGLDYWGQGTLVTVSSASTKGPSVFPLA
PSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTP
EVTCVVVDVSHEDPEVKENWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLSPGK
B29_graft light chain
(SEQ ID NO: 20)
DIQMTQSPSSLSASVGDRVTITCKSSQSLLHSDGKTFLNWFQQKPGKSPKLLIYLVSKLESGVP
SRFSGSGSGTDFTLTISSLQPEDFATYYCLQHTHEPLTFGQGTKVEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSENRGEC
Chimeric _heavy chain
(SEQ ID NO: 22)
QIQLVQSGPELKKPGETVKISCKASGYTFTTYGMSWVKQAPGKGLKWMGWINTYSGMPTYADDF
KGRFAFSLETSATTAYLQINNLKNEDTATYFCARPLLGLDYWGQGTTLTVSSASTKGPSVFPLA
PSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLSPGK
Chimeric_ light chain
(SEQ ID NO: 24)
DIVMTQTPLSLSVTIGQPASISCKSSQSLLHSDGKTFLNWFLQRPGQSPKLLIYLVSKLESGIP
DRFSGSGSGTDFTLKISRVEVEDLGVYYCLQHTHFPLTFGAGTKLEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSENRGEC

### Nucleic Acid Sequences of the Humanized and Chimeric Antibodies

B29_graft heavy chain
(SEQ ID NO: 19)
GCCGCCACCATGGGCTGGTCCCTGATTCTGCTGTTCCTGGTGGCTGTGGCTACCAGGGTGCTGA
GTGAGATCCAGCTGGTGGAGAGCGGAGGAGGACTGGTGCAGCCAGGAGGATCTGTGAGGCTGAG
CTGCGCAGCATCCGGATACAACTTCAAGACCTATGGCATGTCCTGGGTGCGCCAGGCACCAGGC
AAGGGACTGGAGTGGATGGGCTGGATCAATACCTACTCTGGCATGCCTACATATGCCGACGATT
TCAAGGGCCGGTTCACCTTTTCTCTGGACACCAGCAAGAACACAGCCTACCTGCAGATCAATTC
CCTGCGGGCCGAGGACACAGCCGTGTACTTTTGTGCCAGACCCCTGCTGGGCCTGGATTATTGG
GGCCAGGGCACCCTGGTGACAGTGAGCTCCGCTAGCACCAAGGGCCCATCGGTCTTCCCCCTGG
CACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCT
TGGGCACCCAGACCTACATCTGCAACGTGAATCACAAGCCCAGCAACACCAAGGTGGACAAGAA
AGTTGAGCCCAAATCTTGTGACAAAACTCACACATGCCCACCGTGCCCAGCACCTGAACTCCTG
GGGGGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGAGGAGATGACCAAGAACCAGGTC
AGCCTGACCTGCCTGGTCAAAGGCTTCTATCCCAGCGACATCGCCGTGGAGTGGGAGAGCAATG
GGCAGCCGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTAAATGA
B29_graft light chain
(SEQ ID NO: 21)
GCCGCCACCATGGGCTGGTCCTGTATCATCCTGTTCCTGGTGGCTACAGCCACAGGAGTGCATA
GTGACATCCAGATGACACAGTCCCCTAGCTCCCTGAGCGCCTCCGTGGGCGATAGGGTGACCAT
CACATGCAAGTCTAGCCAGTCTCTGCTGCACAGCGACGGCAAGACCTTCCTGAACTGGTTTCAG
CAGAAGCCCGGCAAGTCTCCTAAGCTGCTGATCTACCTGGTGTCCAAGCTGGAGTCTGGCGTGC
CCAGCAGATTCTCTGGCAGCGGCTCCGGCACAGACTTTACCCTGACAATCTCCTCTCTGCAGCC
AGAGGATTTCGCCACCTACTATTGTCTGCAGCACACCCACTTCCCCCTGACATTTGGCCAGGGC
ACCAAGGTGGAGATCAAGCGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGC
CAAAGTACAGTGGAAGGTGGATAACGCCCTCCAATCGGGTAACTCCCAGGAGAGTGTCACAGAG
CAGGACAGCAAGGACAGCACCTACAGCCTCAGCAGCACCCTGACGCTGAGCAAAGCAGACTACG
AGAAACACAAAGTCTACGCCTGCGAAGTCACCCATCAGGGCCTGAGCTCGCCCGTCACAAAGAG
CTTCAACAGGGGAGAGTGTTAG
Chimeric_heavy chain
(SEQ ID NO: 23)
GCCGCCACCATGGGCTGGTCCCTGATTCTGCTGTTCCTGGTGGCTGTGGCTACCAGGGTGCTGA
GTCAGATCCAGCTGGTGCAGTCTGGCCCCGAGCTGAAGAAGCCTGGCGAGACCGTGAAGATCTC
TTGCAAGGCCAGCGGCTACACATTCACCACATATGGCATGAGCTGGGTGAAGCAGGCACCAGGC
AAGGGCCTGAAGTGGATGGGCTGGATCAACACCTACTCCGGCATGCCCACATATGCCGACGATT
TCAAGGGCCGGTTCGCCTTTTCCCTGGAGACCTCTGCCACCACAGCCTACCTGCAGATCAACAA
TCTGAAGAATGAGGACACCGCCACATACTTTTGTGCCAGACCCCTGCTGGGCCTGGATTATTGG
GGCCAGGGCACCACACTGACAGTGAGCTCCGCTAGCACCAAGGGCCCATCGGTCTTCCCCCTGG
CACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCT
TGGGCACCCAGACCTACATCTGCAACGTGAATCACAAGCCCAGCAACACCAAGGTGGACAAGAA
AGTTGAGCCCAAATCTTGTGACAAAACTCACACATGCCCACCGTGCCCAGCACCTGAACTCCTG
GGGGGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGAGGAGATGACCAAGAACCAGGTC
AGCCTGACCTGCCTGGTCAAAGGCTTCTATCCCAGCGACATCGCCGTGGAGTGGGAGAGCAATG
GGCAGCCGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTAAATGA
Chimeric_light chain
(SEQ ID NO: 25)
GCCGCCACCATGGGCTGGTCCTGTATCATCCTGTTCCTGGTGGCTACAGCCACAGGAGTGCATA
GTGACATCGTGATGACCCAGACACCACTGTCTCTGAGCGTGACAATCGGCCAGCCCGCCTCCAT
CTCTTGCAAGAGCTCCCAGAGCCTGCTGCACTCCGATGGCAAGACCTTCCTGAACTGGTTTCTG
CAGAGGCCAGGACAGTCCCCTAAGCTGCTGATCTACCTGGTGTCTAAGCTGGAGAGCGGAATCC
CTGACCGGTTCAGCGGATCCGGATCTGGAACCGACTTCACCCTGAAGATCTCTAGAGTGGAGGT
GGAGGACCTGGGCGTGTACTATTGTCTGCAGCACACCCACTTCCCCCTGACATTTGGCGCCGGC
ACCAAGCTGGAGATCAAGCGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGC
CAAAGTACAGTGGAAGGTGGATAACGCCCTCCAATCGGGTAACTCCCAGGAGAGTGTCACAGAG
CAGGACAGCAAGGACAGCACCTACAGCCTCAGCAGCACCCTGACGCTGAGCAAAGCAGACTACG
AGAAACACAAAGTCTACGCCTGCGAAGTCACCCATCAGGGCCTGAGCTCGCCCGTCACAAAGAG
CTTCAACAGGGGAGAGTGTTAG

TABLE 1
Table of Sequences

| SEQ | | |
|---|---|---|
| ID | | |
| NO | Description | Sequence |
| 1 | B29_graft | GYNFKTY |
| | Heavy chain | |
| | CDR1 - | |
| | amino acid | |
| | (B29) | |
| 2 | B29_chimeric | GYTFTTY |
| | Heavy chain | |
| | CDR1 - | |
| | amino acid | |
| | (B29) | |
| 3 | Heavy chain | GGGTATACCTTCACAACCTAT |
| | CDR1 - | |
| | nucleic acid | |
| | (B29) | |
| 4 | Heavy chain | NTYSGM |
| | CDR2 - | |
| | amino acid | |
| | (B29) | |
| 5 | Heavy chain | AACACCTACTCTGGAATG |
| | CDR2 - | |
| | nucleic acid | |
| | (B29) | |
| 6 | Heavy chain | PLLGLDY |
| | CDR3 - | |
| | amino acid | |
| | (B29) | |
| 7 | Heavy chain | CCTCTCCTGGGACTTGACTAT |
| | CDR3 - | |
| | nucleic acid | |
| | (B29) | |
| 8 | Light chain | KSSQSLLHSDGK |
| | CDR1 - | |
| | amino acid | |
| | (B29) | |
| 9 | Light chain | AAGTCAAGTCAGAGCCTCTTACATAGTGATGGAAAG |
| | CDR1 - | |
| | nucleic acid | |
| | (B29) | |
| 10 | Light chain | LVSKLES |
| | CDR2 - | |
| | amino acid | |
| | (B29) | |
| 11 | Light chain | CTGGTGTCTAAACTGGAATCT |
| | CDR2 - | |
| | nucleic acid | |
| | (B29) | |
| 12 | Light chain | LQHTHFPLT |
| | CDR3 - | |
| | amino acid | |
| | (B29) | |
| 13 | Light chain | TTGCAACATACACATTTTCCGCTCACG |
| | CDR3 - | |
| | nucleic acid | |
| | (B29) | |
| 14 | B29 Heavy | QIQLVQSGPELKKPGETVKISCKASGYTFTTYGMSWVKQAPGKGLKWMG |
| | Chain - | WINTYSGMPTYADDFKGRFAFSLETSATTAYLQINNLKNEDTATYFCAR |
| | amino acid | PLLGLDYWGQGTTLTVSS |
| 15 | B29 Heavy | CAGATCCAGTTGGTACAGTCTGGACCTGAGCTGAAGAAGCCTGGAGAGA |
| | Chain - | CAGTCAAGATCTCCTGCAAGGCCTCCGGGTATACCTTCACAACCTATGG |
| | nucleic acid | AATGAGCTGGGTGAAACAGGCTCCAGGAAAGGGTTTAAAGTGGATGGGC |
| | | TGGATAAACACCTACTCTGGAATGCCAACATATGCTGATGACTTCAAGG |
| | | GACGGTTTGCCTTCTCTTTGGAAACCTCTGCCACCACTGCCTATTTGCA |
| | | GATCAACAACCTCAAAAATGAGGACACGGCTACATATTTCTGTGCAAGA |
| | | CCTCTCCTGGGACTTGACTATTGGGGCCAAGGCACCACTCTCACAGTCT |
| | | CCTCA |
| 16 | B29 Light | DIVMTQTPLSLSVTIGQPASISCKSSQSLLHSDGKTFLNWFLQRPGQSP |
| | Chain - | KLLIYLVSKLESGIPDRFSGSGSGTDFTLKISRVEVEDLGVYYCLQHTH |
| | amino acid | FPLTFGAGTKLEIK |
| 17 | B29 Light | GATATTGTGATGACCCAGACTCCACTGTCTTTGTCGGTTACCATTGGAC |
| | Chain - | AACCAGCCTCCATCTCTTGCAAGTCAAGTCAGAGCCTCTTACATAGTGA |
| | nucleic acid | TGGAAAGACATTTTTGAATTGGTTTTTACAGAGGCCAGGCCAGTCTCCA |
| | | AAGCTCCTAATCTATCTGGTGTCTAAACTGGAATCTGGCATCCCTGACA |
| | | GGTTCAGTGGCAGTGGATCAGGGACAGATTTCACACTGAAAATCAGCAG |
| | | AGTGGAGGTTGAGGATTTGGGAGTTTATTACTGCTTGCAACATACACAT |
| | | TTTCCGCTCACGTTCGGTGCTGGGACCAAACTGGAAATAAAA |
| 18 | B29_graft | EIQLVESGGGLVQPGGSVRLSCAASGYNFKTYGMSWVRQAPGKGLEWMG |
| | heavy chain | WINTYSGMPTYADDFKGRFTFSLDTSKNTAYLQINSLRAEDTAVYFCAR |
| | Amino acid | PLLGLDYWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQT |
| | | YICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQKSLS |
| | | LSPGK |
| 19 | B29_graft | GCCGCCACCATGGGCTGGTCCCTGATTCTGCTGTTCCTGGTGGCTGTGG |
| | heavy chain | CTACCAGGGTGCTGAGTGAGATCCAGCTGGTGGAGAGCGGAGGAGGACT |
| | nucleic acid | GGTGCAGCCAGGAGGATCTGTGAGGCTGAGCTGCGCAGCATCCGGATAC |
| | | AACTTCAAGACCTATGGCATGTCCTGGGTGCGCCAGGCACCAGGCAAGG |
| | | GACTGGAGTGGATGGGCTGGATCAATACCTACTCTGGCATGCCTACATA |
| | | TGCCGACGATTTCAAGGGCCGGTTCACCTTTTCTCTGGACACCAGCAAG |
| | | AACACAGCCTACCTGCAGATCAATTCCCTGCGGGCCGAGGACACAGCCG |
| | | TGTACTTTTGTGCCAGACCCCTGCTGGGCCTGGATTATTGGGGCCAGGG |
| | | CACCCTGGTGACAGTGAGCTCCGCTAGCACCAAGGGCCCATCGGTCTTC |
| | | CCCCTGGCACCCTCCTCCAAGAGCACCTCTGGGGGCACAGCGGCCCTGG |
| | | GCTGCCTGGTCAAGGACTACTTCCCCGAACCGGTGACGGTGTCGTGGAA |
| | | CTCAGGCGCCCTGACCAGCGGCGTGCACACCTTCCCGGCTGTCCTACAG |
| | | TCCTCAGGACTCTACTCCCTCAGCAGCGTGGTGACCGTGCCCTCCAGCA |
| | | GCTTGGGCACCCAGACCTACATCTGCAACGTGAATCACAAGCCCAGCAA |
| | | CACCAAGGTGGACAAGAAAGTTGAGCCCAAATCTTGTGACAAAACTCAC |
| | | ACATGCCCACCGTGCCCAGCACCTGAACTCCTGGGGGGACCGTCAGTCT |
| | | TCCTCTTCCCCCCAAAACCCAAGGACACCCTCATGATCTCCCGGACCCC |
| | | TGAGGTCACATGCGTGGTGGTGGACGTGAGCCACGAAGACCCTGAGGTC |
| | | AAGTTCAACTGGTACGTGGACGGCGTGGAGGTGCATAATGCCAAGACAA |
| | | AGCCGCGGGAGGAGCAGTACAACAGCACGTACCGTGTGGTCAGCGTCCT |
| | | CACCGTCCTGCACCAGGACTGGCTGAATGGCAAGGAGTACAAGTGCAAG |
| | | GTCTCCAACAAAGCCCTCCCAGCCCCCATCGAGAAAACCATCTCCAAAG |
| | | CCAAAGGGCAGCCCCGAGAACCACAGGTGTACACCCTGCCCCCATCCCG |
| | | GGAGGAGATGACCAAGAACCAGGTCAGCCTGACCTGCCTGGTCAAAGGC |
| | | TTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTT |
| | | CTTCCTCTACAGCAAGCTCACCGTGGACAAGAGCAGGTGGCAGCAGGGG |
| | | AACGTCTTCTCATGCTCCGTGATGCATGAGGCTCTGCACAACCACTACA |
| | | CGCAGAAGAGCCTCTCCCTGTCTCCGGGTAAATGA |
| | | Bold regions correspond to CDRs |
| | | First bold region corresponds to CDR1 |
| | | Second bold region corresponds to CDR2 |
| | | Third bold region corresponds to CDR3 |
| 20 | B29_graft | DIQMTQSPSSLSASVGDRVTITCKSSQSLLHSDGKTFLNWFQQKPGKSP |
| | light chain | KLLIYLVSKLESGVPSRFSGSGSGTDFTLTISSLQPEDFATYYCLQHTH |
| | | FPLTFGQGTKVEIKRTVAAPSVFIFPPSDEQLKSGTASVVCLLNNFYPR |
| | | EAKVQWKVDNALQSGNSQESVTEQDSKDSTYSLSSTLTLSKADYEKHKV |
| | | YACEVTHQGLSSPVTKSFNRGEC |
| 21 | B29_graft | GCCGCCACCATGGGCTGGTCCTGTATCATCCTGTTCCTGGTGGCTACAG |
| | light chain | CCACAGGAGTGCATAGTGACATCCAGATGACACAGTCCCCTAGCTCCCT |
| | | GAGCGCCTCCGTGGGCGATAGGGTGACCATCACATGCAAGTCTAGCCAG |
| | | TCTCTGCTGCACAGCGACGGCAAGACCTTCCTGAACTGGTTTCAGCAGA |
| | | AGCCCGGCAAGTCTCCTAAGCTGCTGATCTACCTGGTGTCCAAGCTGGA |
| | | GTCTGGCGTGCCCAGCAGATTCTCTGGCAGCGGCTCCGGCACAGACTTT |
| | | ACCCTGACAATCTCCTCTCTGCAGCCAGAGGATTTCGCCACCTACTATT |
| | | GTCTGCAGCACACCCACTTCCCCCTGACATTTGGCCAGGGCACCAAGGT |
| | | GGAGATCAAGCGTACGGTGGCTGCACCATCTGTCTTCATCTTCCCGCCA |
| | | TCTGATGAGCAGTTGAAATCTGGAACTGCCTCTGTTGTGTGCCTGCTGA |
| | | ATAACTTCTATCCCAGAGAGGCCAAAGTACAGTGGAAGGTGGATAACGC |
| | | CCTCCAATCGGGTAACTCCCAGGAGAGTGTCACAGAGCAGGACAGCAAG |
| | | GACAGCACCTACAGCCTCAGCAGCACCCTGACGCTGAGCAAAGCAGACT |
| | | ACGAGAAACACAAAGTCTACGCCTGCGAAGTCACCCATCAGGGCCTGAG |
| | | CTCGCCCGTCACAAAGAGCTTCAACAGGGGAGAGTGTTAG |
| | | Bold regions correspond to CDRs |
| | | First bold region corresponds to CDR1 |
| | | Second bold region corresponds to CDR2 |
| | | Third bold region corresponds to CDR3 |
| 22 | Chimeric_heavy | QIQLVQSGPELKKPGETVKISCKASGYTFTTYGMSWVKQAPGKGLKWMG |
| | chain | WINTYSGMPTYADDFKGRFAFSLETSATTAYLQINNIKNEDTATYFCAR |
| | | PLLGLDYWGQGTTLTVSSASTKGPSVFPLAPSSKSTSGGTAALGCLVKD |
| | | YFPEPVTVSWNSGALTSGVHTFPAVLQSSGLYSLSSVVTVPSSSLGTQT |
| | | YICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQKSLS |
| | | LSPGK |
| 23 | Chimeric_heavy | GCCGCCACCATGGGCTGGTCCCTGATTCTGCTGTTCCTGGTGGCTGTGG |
| | chain | CTACCAGGGTGCTGAGTCAGATCCAGCTGGTGCAGTCTGGCCCCGAGCT |
| | | GAAGAAGCCTGGCGAGACCGTGAAGATCTCTTGCAAGGCCAGCGGCTAC |
| | | ACATTCACCACATATGGCATGAGCTGGGTGAAGCAGGCACCAGGCAAGG |
| | | GCCTGAAGTGGATGGGCTGGATCAACACCTACTCCGGCATGCCCACATA |
| | | TGCCGACGATTTCAAGGGCCGGTTCGCCTTTTCCCTGGAGACCTCTGCC |
| | | ACCACAGCCTACCTGCAGATCAACAATCTGAAGAATGAGGACACCGCCA |
| | | CATACTTTTGTGCCAGACCCCTGCTGGGCCTGGATTATTGGGGCCAGGG |
| | | CACCACACTGACAGTGAGCTCCGCTAGCACCAAGGGCCCATCGGTCTTC |
| | | CCCCTGGCACCCTCCTCCAAGAGCACCTCTGGGGGCACAGCGGCCCTGG |
| | | GCTGCCTGGTCAAGGACTACTTCCCCGAACCGGTGACGGTGTCGTGGAA |
| | | CTCAGGCGCCCTGACCAGCGGCGTGCACACCTTCCCGGCTGTCCTACAG |
| | | TCCTCAGGACTCTACTCCCTCAGCAGCGTGGTGACCGTGCCCTCCAGCA |
| | | GCTTGGGCACCCAGACCTACATCTGCAACGTGAATCACAAGCCCAGCAA |
| | | CACCAAGGTGGACAAGAAAGTTGAGCCCAAATCTTGTGACAAAACTCAC |
| | | ACATGCCCACCGTGCCCAGCACCTGAACTCCTGGGGGGACCGTCAGTCT |
| | | TCCTCTTCCCCCCAAAACCCAAGGACACCCTCATGATCTCCCGGACCCC |
| | | TGAGGTCACATGCGTGGTGGTGGACGTGAGCCACGAAGACCCTGAGGTC |
| | | AAGTTCAACTGGTACGTGGACGGCGTGGAGGTGCATAATGCCAAGACAA |
| | | AGCCGCGGGAGGAGCAGTACAACAGCACGTACCGTGTGGTCAGCGTCCT |
| | | CACCGTCCTGCACCAGGACTGGCTGAATGGCAAGGAGTACAAGTGCAAG |
| | | GTCTCCAACAAAGCCCTCCCAGCCCCCATCGAGAAAACCATCTCCAAAG |
| | | CCAAAGGGCAGCCCCGAGAACCACAGGTGTACACCCTGCCCCCATCCCG |
| | | GGAGGAGATGACCAAGAACCAGGTCAGCCTGACCTGCCTGGTCAAAGGC |
| | | TTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTT |
| | | CTTCCTCTACAGCAAGCTCACCGTGGACAAGAGCAGGTGGCAGCAGGGG |
| | | AACGTCTTCTCATGCTCCGTGATGCATGAGGCTCTGCACAACCACTACA |
| | | CGCAGAAGAGCCTCTCCCTGTCTCCGGGTAAATGA |
| | | Bold regions correspond to CDRs: |
| | | First bold region corresponds to CDR1 |
| | | Second bold region corresponds to CDR2 |
| | | Third bold region corresponds to CDR3 |
| 24 | Chimeric_light | DIVMTQTPLSLSVTIGQPASISCKSSQSLLHSDGKTFLNWFLQRPGQSP |
| | chain | KLLIYLVSKLESGIPDRFSGSGSGTDFTLKISRVEVEDLGVYYCLQHTH |
| | | FPLTFGAGTKLEIKRTVAAPSVFIFPPSDEQLKSGTASVVCLLNNFYPR |
| | | EAKVQWKVDNALQSGNSQESVTEQDSKDSTYSLSSTLTLSKADYEKHKV |
| | | YACEVTHQGLSSPVTKSFNRGEC |
| 25 | Chimeric_light | GCCGCCACCATGGGCTGGTCCTGTATCATCCTGTTCCTGGTGGCTACAG |
| | chain | CCACAGGAGTGCATAGTGACATCGTGATGACCCAGACACCACTGTCTCT |
| | | GAGCGTGACAATCGGCCAGCCCGCCTCCATCTCTTGCAAGAGCTCCCAG |
| | | AGCCTGCTGCACTCCGATGGCAAGACCTTCCTGAACTGGTTTCTGCAGA |
| | | GGCCAGGACAGTCCCCTAAGCTGCTGATCTACCTGGTGTCTAAGCTGGA |
| | | GAGCGGAATCCCTGACCGGTTCAGCGGATCCGGATCTGGAACCGACTTC |
| | | ACCCTGAAGATCTCTAGAGTGGAGGTGGAGGACCTGGGCGTGTACTATT |
| | | GTCTGCAGCACACCCACTTCCCCCTGACATTTGGCGCCGGCACCAAGCT |
| | | GGAGATCAAGCGTACGGTGGCTGCACCATCTGTCTTCATCTTCCCGCCA |
| | | TCTGATGAGCAGTTGAAATCTGGAACTGCCTCTGTTGTGTGCCTGCTGA |
| | | ATAACTTCTATCCCAGAGAGGCCAAAGTACAGTGGAAGGTGGATAACGC |
| | | CCTCCAATCGGGTAACTCCCAGGAGAGTGTCACAGAGCAGGACAGCAAG |
| | | GACAGCACCTACAGCCTCAGCAGCACCCTGACGCTGAGCAAAGCAGACT |
| | | ACGAGAAACACAAAGTCTACGCCTGCGAAGTCACCCATCAGGGCCTGAG |
| | | CTCGCCCGTCACAAAGAGCTTCAACAGGGGAGAGTGTTAG |
| | | Bold regions correspond to CDRs: |
| | | First bold region corresponds to CDR1 |
| | | Second bold region corresponds to CDR2 |
| | | Third bold region corresponds to CDR3 |
| 26 | B29_graft | TYGMS |
| | Heavy chain | |
| | CDR1 - | |
| | amino acid | |
| | (B29) | |
| 27 | B29 graft | WINTYSGMPTYADDFKG |
| | Heavy chain | |
| | CDR2 - | |
| | amino acid | |
| | (B29) | |
| 28 | B29 graft | PLLGLDY |
| | Heavy chain | |
| | CDR3 - | |
| | amino acid | |
| | (B29) | |
| 29 | Light chain | KSSQSLLHSDGKTFLN |
| | CDR1 - | |
| | amino acid | |
| | (B29) | |

### Example 3

Regulation of the equilibrium between proteases and their inhibitors is fundamental to the survival of multicellular organisms. The immunological response to serpinB13, which is a protease inhibitor of cathepsin L (catL), plays an important role in slowing down, and preventing the development, of insulin-dependent diabetes. This hypothesis is rooted in translational and experimental animal studies. Specifically, previous analysis of baseline autoantibody (AA) activity to serpinB13 in first-degree relatives of type 1 diabetes (TID) probands during their enrollment in the DPT-1 prevention trial revealed a significantly lower incidence of diabetes in individuals with detectable anti-serpin activity compared with subjects negative for anti-serpin activity. Moreover, studies in animals demonstrated that injecting a monoclonal antibody (mAb) to serpinB13 led to a robust increase in the number of pancreatic Ngn3+ progenitor cells, a significant expansion of islet mass, and improved resistance to severe diabetes in adulthood.

SerpinB13 AA is examined in a cohort of DPT-1 subjects with an emphasis on samples that were collected during the follow-up period rather than at baseline. Intermediate-to-high risk individuals are studied and the patterns of serological binding activity to serpinB13 over time is assessed along with their association with progression to clinical diabetes.

Regulation of the Notch pathway, a highly conserved signaling pathway that restricts generation of Ngn3⁺ endocrine progenitors is examined. Using pancreatic embryonic explants, it is examined whether human serpinB13 AA and humanized mAb to serpinB13 induce Ngn3⁺ cells via catL-mediated cleavage of Notch receptors and subsequent disruption of Notch signaling.

### Example 4

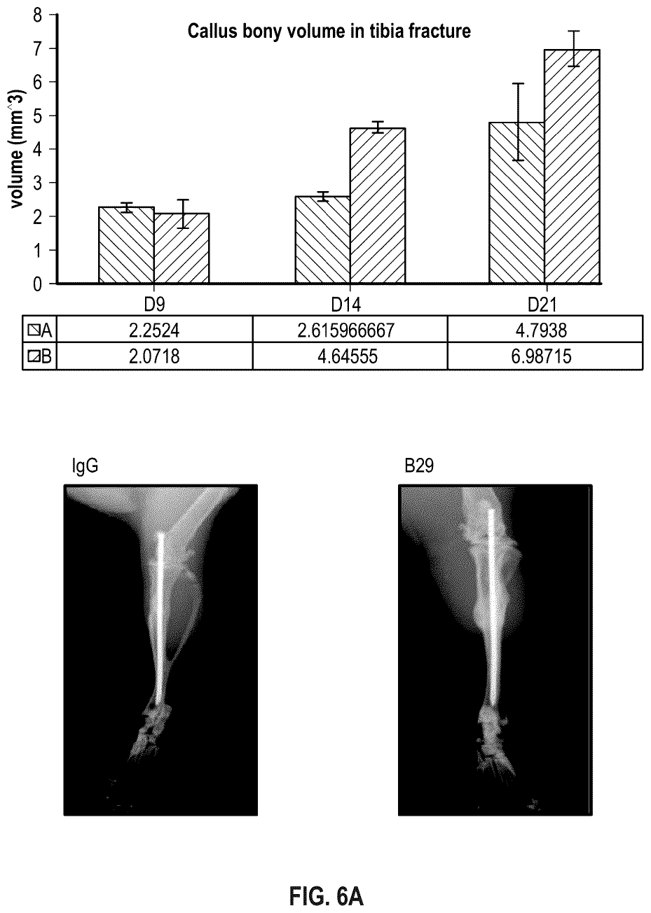

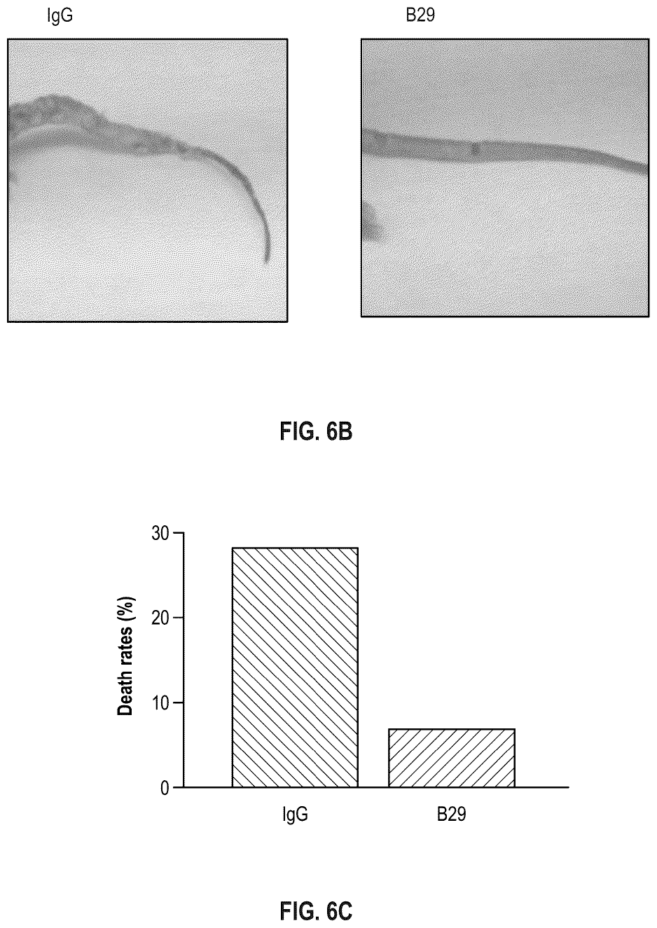

The impact of mouse monoclonal antibody to serpin B13 (clone B29) on tissue regeneration in different organs was studied. FIGS. 6A-6C.

### Example 5

The inventors identified a novel autoantibody (AA) to serpin B13, a protease inhibitor of cathepsin L (catL). Quite unexpectedly, when using human samples from several consortia it was found that, unlike other AAs in TID, serpinB13 AA was associated with improved outcomes. In particular, recent examination of healthy individuals at risk for type 1 diabetes (TID), and who had been enrolled in the Diabetes Prevention Trial for Type 1 Diabetes (DPT-1), revealed significant benefits for those who were positive for anti-serpinB13 activity at baseline. These subjects demonstrated a lower rate of progression to the clinical onset of TID, and their overall incidence of diabetes by the end of a seven-year follow-up was also lower compared with individuals who were negative at baseline for serpinB13 AA. In addition to these translational studies, a novel mouse monoclonal antibody (mAb [clone B29]) was developed, which was used as a model to examine the potential functionality of the immunological response to serpinB13. Studies with this antibody showed that neutralizing the serpinB13 molecule augmented catL activity, increased the number of pancreatic endocrine progenitor cells expressing neurogenin 3 (Ngn3), and ultimately helped to prevent severe diabetes. Together, the studies in humans and mice suggest that serpinB13 AA is a biomarker of improved islet biology. Since activation of the Notch pathway leads to inhibition of Ngn3 expression, it is hypothesized that catL, induced by antibody-mediated neuralization of serpinB13, helps to reverse this repression b impairing Notch function.

Approximately 60% of the patients with chronic pancreatitis who receive islet autotransplant remain insulin dependent after surgery, while 40% become insulin independent. While islet mass transplanted is an important predictor of insulin independence, other factors that influence this outcome remain elusive. The impact of baseline (pre-transplant) serpinB13 AA in these patients is tested. Specifically, serpinB13 AA expression is examined for its potential association with insulin dependence (on/off insulin), insulin dose, and fasting and stimulated C-peptide levels at 1 year after islet autotransplant. In addition, the association of serpinB13 AA detected in the sera of patients with pancreatitis is examined, with an in vitro function of the islets isolated from the same patients (e.g., the islets from strong and weak secretors of serpinB13 AA, for β-cell proliferation, apoptosis, insulin secretion, the presence of Ngn3+ endocrine progenitors, and expression of genes associated with islet cell regeneration are examined). SerpinB13 AA is examined using a Luminex methodology.

Recently, several fully human mAbs to serpinB13 were developed that maintain binding to the target at similar levels compared with the mouse mAb, clone B29 (discussed above). Gene expression and protein levels of Ngn3 following inhibition of serpinB13 with human anti-serpinB13 mAb is examined. The impact of Notch and catL on the response to human antiserpinB13 mAb is assessed using transgenic mice that either express the constitutively active Notch intracellular domain or are genetically deficient for catL. Finally, the degradomic profile of Notch following induction of catL with human anti-serpinB13 mAb is examined. The studies examine the biological impact of endogenous anti-serpinB13 activity on islet biology on case-by-case basis in humans and, leverage the development of passive immunization with anti-serpinB13 mAb as an approach to reduce the incidence of diabetes after TPIAT. In addition, these studies help to determine whether anti-serpin activity provides an important function by dismantling Notch signaling, thereby allowing a more efficient generation of the endocrine progenitor cells. Ultimately, the findings are applicable to the development of new therapeutic interventions in diabetes and other diseases with a deregulated Notch pathway.

Protease activity is critical for the survival of multicellular organisms. It is not surprising, therefore that proteases are modulated by a number of inhibitors, which themselves are regulated. The research focuses on a fundamental problem of regulation of the balance between proteases and their inhibitors, and its role in islet biology and diabetes). It has been discovered that proteases are key players in regulation of molecules that haven been linked to development and increased regenerative potential in insulin-producing cells, and that by doing so they contribute to better clinical outcomes in diabetes. Specifically, in both human and mouse a novel autoantibody (AA) to serpinB13 protease inhibitor of cathepsin L (catL) has been identified and it was found that this immunological response blocks inhibitory function of serpin, thereby allowing the protease activity of catL to increase. It is believed that low level extracellular catL, through the cleavage of several distinct molecules expressed on the cell-surface in the pancreas positively influences regenerative potential of islet cells thereby offering a lead for therapy development in patients with type 1 diabetes (TID) and other settings that would benefit from improved biology of β-cells, e.g., in the setting of islet autotransplantation that is offered to patients with painful pancreatitis undergoing total pancreatectomy.

SerpinB13. SerpinB13 is a member of the clade B family of potent cysteine and serine protease inhibitors. It is expressed in the exocrine pancreatic ducts and several other tissues. Although clade B serpins are mainly intracellular, serpinB1 (a close relative of serpinB13) has been observed to be released from keratinocytes exposed to UVB light. In addition, serpinB13 functions in the extracellular matrix to suppress angiogenesis, indicating that these serpins can be released under certain conditions. It has been shown that serpinB13 can reach the extracellular milieu during culture of embryonic pancreas explants. Ultimately, release of clade B serpins from cells facilitate induction of an AA response against these molecules.

Anti-serpinB13 activity is a modifying protective factor that actively contributes to protecting pancreatic islets. This is in sharp contrast to many other AAs associated with TID, which are assumed to be predominantly biomarkers of pathological changes in pancreatic islets during development of TID. The idea that stimulating protease activity promotes β-cell regenerative changes through impeded Notch signaling is original. Autoantibody response to serpinB13 is a biomarker of improved clinical outcome in human TID. To assess whether serpinB13 AA promotes β-cell health in humans, the association of this antibody response with residual β-cell function in children with a recent diagnosis of TID was examined, and who were previously enrolled as placebo subjects in one of several Type 1 Diabetes TrialNet double-blind placebo-controlled intervention protocols.

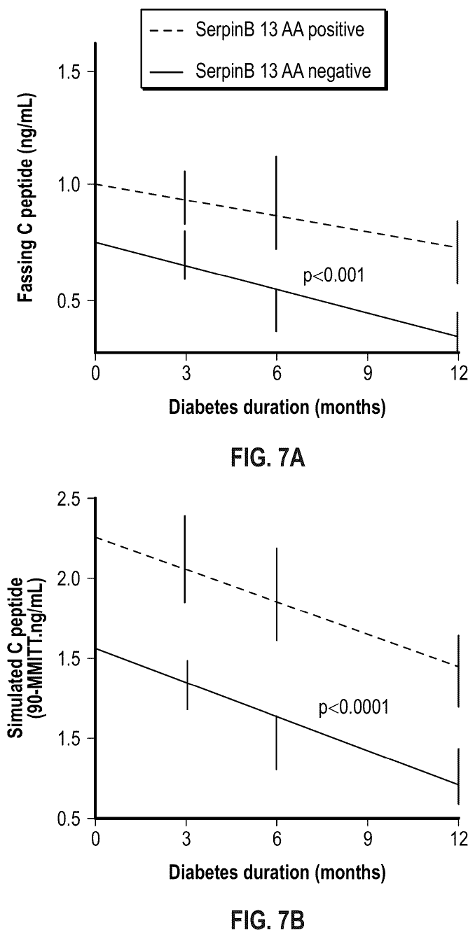

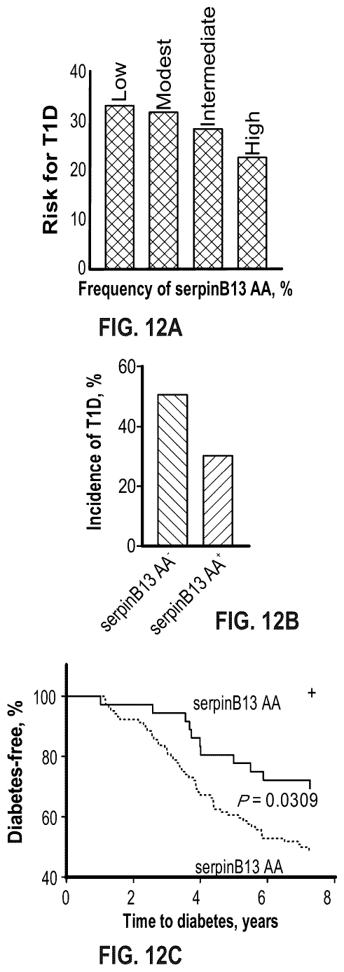

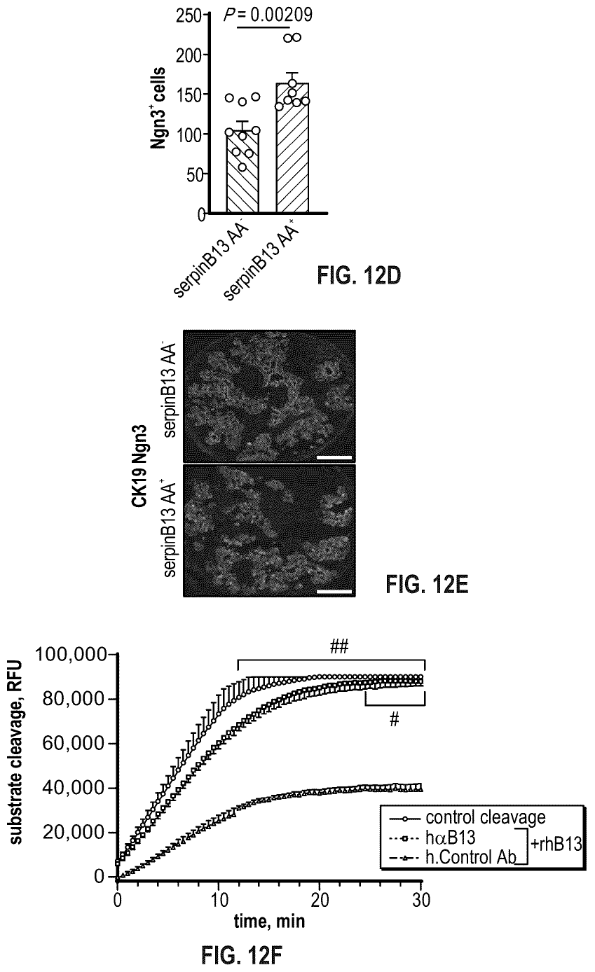

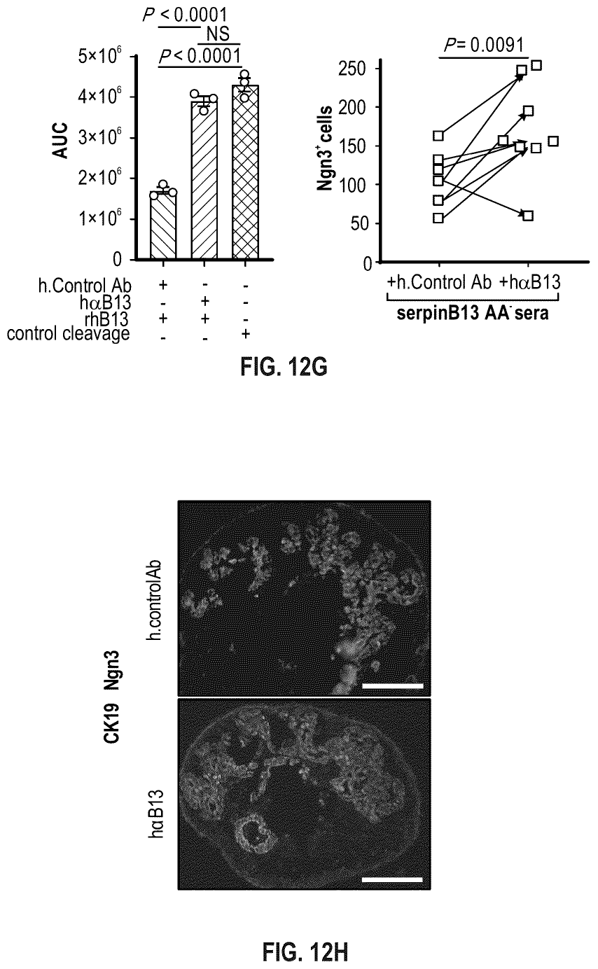

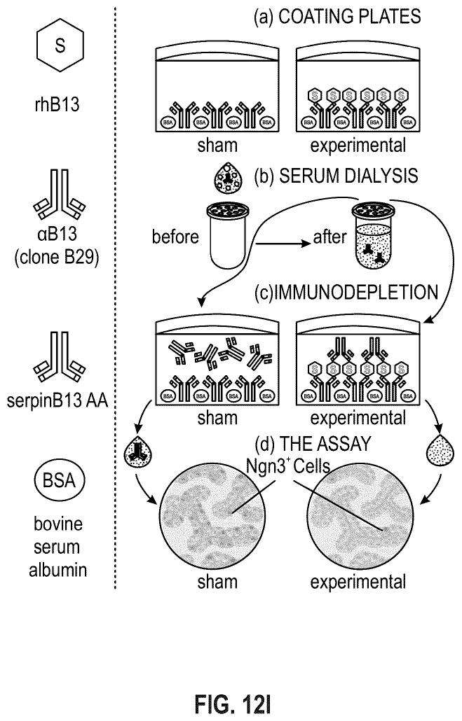

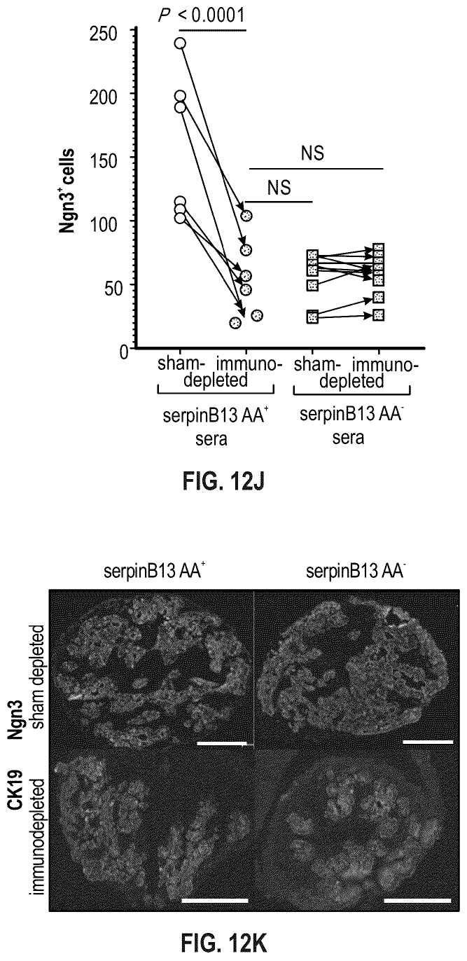

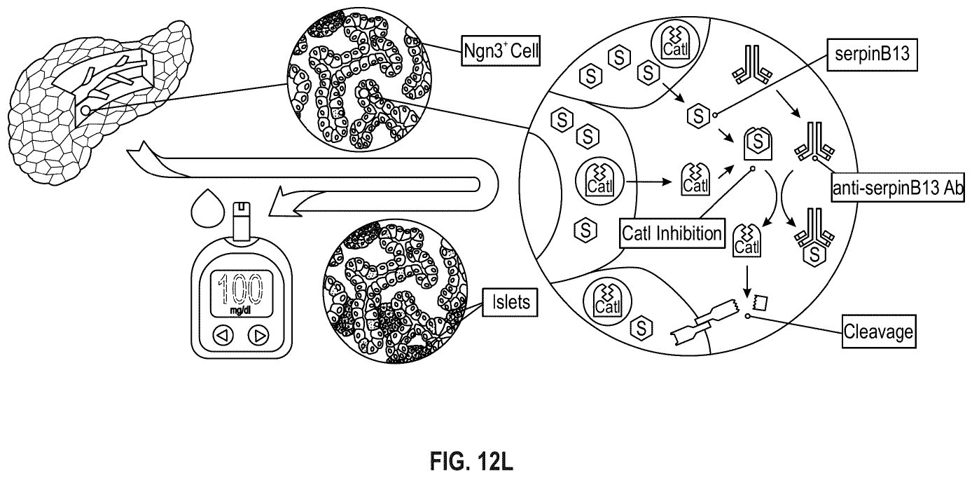

It was found that subjects with serpinB13 AAs had higher fasting and stimulated Cpeptide levels during the first-year post-diagnosis, compared with serpinB13 AA-negative subjects (FIGS. 7A and 7B). Remarkably, the results are very similar to those published by Herold et al. on CD3 mAb, the gold standard in research on novel immunointerventions in TID (Herold et al., Teplizumab (anti-CD3 mAb) treatment preserves C-peptide responses in patients with new-onset type 1 diabetes in a randomized controlled trial. Diabetes 62:3766-3774, 2013.). It was then investigated whether serpinB13 AA also influences the pre-diabetes period and progression to TID. To address this question, serum from individuals who had been enrolled in the DPT-1 study was examined. In baseline samples, a strong significant correlation with diabetes-free status was found. SerpinB13 AA frequency inversely correlated with risk for TID (FIG. 12A), fewer individuals positive for serpinB13 AA developed diabetes (FIG. 12B) and developed it later (FIG. 12C), compared to serpinB13 AA-negative subjects. Therefore, it is believed that serpinB13 AA offers a high level of islet protection.

Cathepsin L protease activity is upregulated following inhibition of serpinB13 with a mAb (clone B29). CatL has been implicated as a serpinB13protease target. To examine the consequences of inhibiting serpinB13 using a mAb (clone B29), the catalytic activity of catL was measured. First, it was noted that serpinB13 mAb dose-dependently enhanced the protease activity of catL when added to a cell extract from pancreatic tissue in vitro. To measure catL activity in vivo, the activity-based probe, ProSense 680, was used. A significant increase in catL activity was observed in the intact pancreas following injection of anti-serpinB13 mAb into mice. This increase was limited to the pancreas in wild-type Balb/c mice and was not observed in the liver, where serpinB13 is not expressed, or in the pancreas of catL-deficient Balb/c mice. Together, these data show that mAb-mediated inhibition of serpinB13 influences the activity of catL in vivo and provides a reliable model that will allow us to examine the role of catL protease activity in islet biology in more detail.

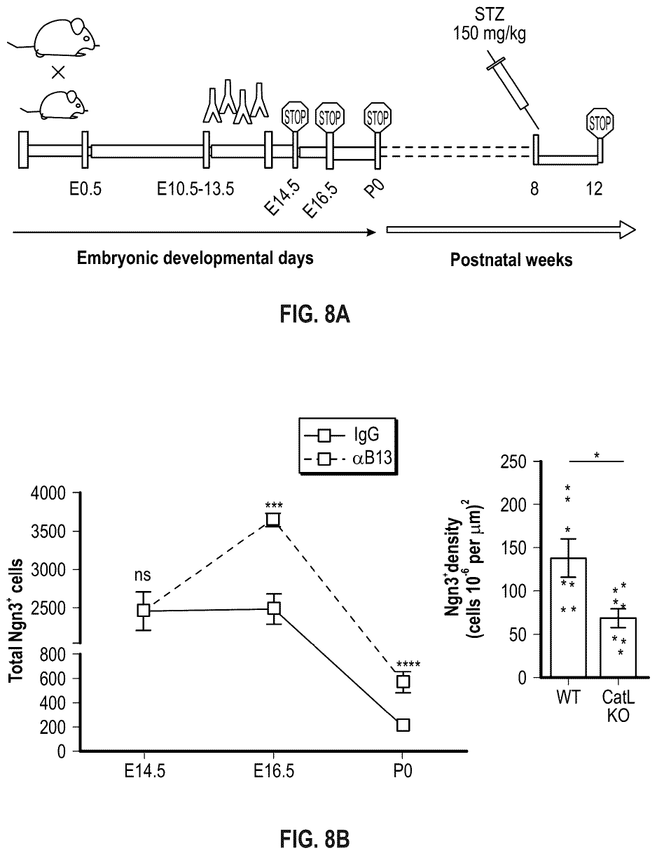

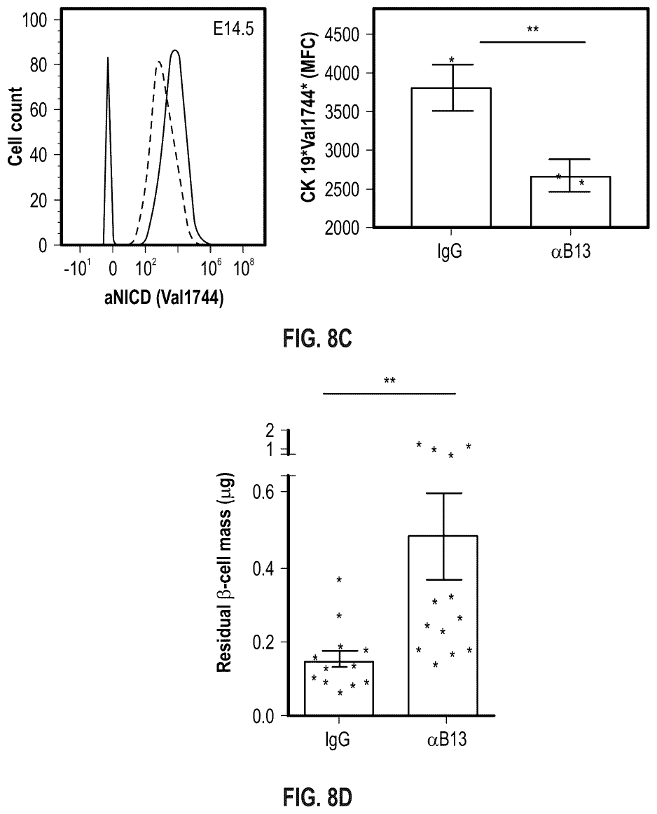

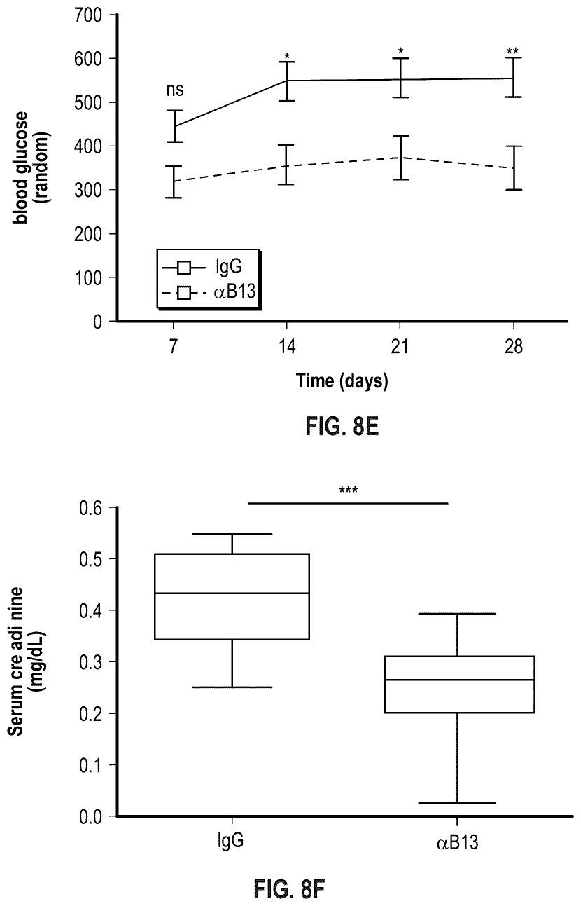

Exposure to anti-serpinB13mAb increases the number of Ngn3+ endocrine progenitor cells in the pancreas and improves outcomes in mouse models of diabetes. To better understand the role of interplay between serpinB13 and catL, and to identify potential novel targets of cleavage by catL that may be relevant to diabetes development, the effect of anti-serpinB13 mAb on the development, of the endocrine pancreas was studied using the protocol depicted in FIG. 8A. It was found that inhibiting serpinB13 with serpinB13 mAb (clone B29) caused a significant increase in the number of Ngn3+ cells in the embryonic pancreas (FIG. 8B), while a genetic deficiency of CatL decreased this cell population (FIG. 8B insert). This effect was observed over a wide range of mAb concentrations and was specific for Ngn3+ cells as the number of CK19+ epithelial cells remained unchanged. Moreover, the level of active Notch intracellular domain (NICD), a fragment of the Notch receptor that restricts endocrine cell development and β-cell function, was diminished in the antibody treated group, compared with the control group (FIG. 8C). These data suggest that CatL can antagonize Notch signaling so that serpinB13 AA may enhance endocrine cell development by enhancing catL activity in the pancreas.

To assess the long-term impact of developmental changes induced by inhibiting serpinB13, newborn mice from Balb/c mothers were followed that received anti-serpinB13 mAb during pregnancy. Prenatal exposure to this mAb led to a significant increase in the number of pancreatic islets and total β-cell number, although the total pancreas and body weight at birth and in adulthood remained the same in the two groups. Of note, prenatal exposure to serpinB13 mAb led to a striking increase in postnatal β-cell mass in the setting of STZ-induced diabetes in adulthood, with a higher preserved residual β-cell mass (FIG. 8D) and reduced severity of disease (FIGS. 8E-8F). Therefore, the studies show that an increase in the number of Ngn3+ endocrine progenitors following inhibition of serpinB13 offers a clinically significant long-term benefit against abrupt loss of insulin-producing cells later in life.

Human anti-serpinB13 mAbs bind specifically to serpinB13, but not to other members of the clade B protein family. To produce human antibody to serpinB13, three rounds of biopanning were performed, using Fab library (1×10¹¹ antibody specificities), against recombinant human serpinB13 produced in baculovirus and immobilized on solid matrix. Screening of individual clones by ELISA led to DNA fingerprinting and sequencing of three unique clones. Positive antibodies were tested by ELISA for binding to the target antigen and cross-reactivity with other proteins. The Fab fragments were then reformatted into fully human full-length IgG1. This approach resulted in generation of three novel fully human mAbs (mAbs1, mAb2 and mAb3), which specifically recognize human serpinB13 but no other serpins of clade B family. It was also noticed that these mAbs recognize both mouse and human serpinB13. The affinity validation by surface plasmon resonance indicated that the novel human antibodies had comparable affinity to the mouse mAb, clone B29.

### Materials and Methods

Cell proliferation: Cy5-azide is used to measure thymidine analogue (e.g., 5-Edu) incorporation during DNA synthesis by the islets. Alternatively, the cells are stained with anti-Ki67 antibody. The islets are cultured in vitro for 24 to 72 hours. These analyses are performed in both insulin-positive and insulin-negative islet cells.

Apoptosis: Cell death in the islets is assessed using the transferase-mediated dUTP nick end-labeling (TUNEL) on rehydrated and trypsin-predigested islet sections, or alternatively by flow cytometry using staining of islet cell suspensions with Violet Annexin C/Dead Cell Apoptosis kit. Islet sections or cell suspensions are co-stained with anti-insulin or anti-glucagon antibodies to examine individual endocrine subtypes.

Insulin content and secretion: After overnight incubation, groups of islets (5 per sample) are preincubated in Krebs-Ringer bicarbonate buffer supplemented with 0.5% BSA, then stimulated with 5 or 25 mM glucose in the same buffer for 60 min. at 37° C. Following glucose stimulation, the media is collected, and secreted insulin is evaluated using the ELISA kit (Mercodia). To determine insulin content inside the islets, the islets are treated with 0.1 mL acidified ethanol and kept frozen until ELISA for insulin.

Gene expression: Quantitative RT-PCR analysis is performed to monitor expression of genes that (1) drive cells toward the endocrine lineage (e.g., Ngn3, insulinoma1, and NeuroD1/β1), (2) act as beta-cell differentiation factors (Pdx1, Pax4, NeuroD1/β2, MafA, Nkx6.1, and Nkx2.2), (3) help regulate expression of insulin (Pdx1, MafA, β2, and Nkx2.2), and (4) participate in β-cell proliferation (Pax4). In addition, Reg genes that are expressed in the regenerating islet tissue following subtotal pancreatectomy are examined. The RT-PCR data is confirmed by Western blot analysis for genes that show the most reproducible and prominent changes.

### Example 6

### SerpinB13 Antibodies Promote β-Cell Development and Resistance to Type 1 Diabetes

Endocrine cell development is dependent on the rescue of neurogenin3 (Ngn3) transcription factor from repression by Notch. The signals that prevent Notch signaling, allowing the formation of pancreatic endocrine cells, remain unclear. We show that inhibiting serpinB13, a cathepsin L (CatL) protease inhibitor expressed in the pancreatic epithelium, causes cleavage of the extracellular domain of Notch 1. This is followed by a two-fold increase in Ngn3⁺ progenitor cell population and enhanced conversion of these cells to express insulin. Conversely, both recombinant serpinB13 protein and CatL-deficiency downregulate Ngn3⁺ cell output. The embryonic exposure to inhibitory anti-serpinB13 antibody results in increased islet cell mass and improved outcomes in streptozotocin-induced diabetes after birth. Moreover, anti-serpinB13 autoantibodies (AAs) impede progression to type 1 diabetes (TID) in children and stimulate Ngn3⁺ endocrine progenitor formation in the pancreas. These data demonstrate long-term impact of serpinB13 activity on islet biology and suggest that promoting protease activity by blocking this serpin has therapeutic potential in TID.

SerpinB13 is an inhibitor of cathepsin L (CatL) and a member of the clade B serpins, a protein family that plays a critical role in limiting tissue injury by inhibiting proteinases, either expressed in the host or derived from microbes and parasites. Based on the critical role that proteases play in inducing tissue patterning signals during embryogenesis, additional important roles could be hypothesized for the clade B serpins. For example, inhibition of a protease that shares a similarity with CatL results in inhibition of dorsoventral polarity in Xenophus embryos. However, whether the interplay between CatL and serpinB13 modulate tissue patterning in the pancreas, and to what extent this potential role may be exploited for the benefit of humans with decreased insulin-producing cells (e.g., in type 1 diabetes [TID] patients, or those who are clinically healthy but at risk for this disease), remains unknown.

The Notch signaling pathway is a highly conserved developmental pathway that is important in pancreatic development and growth, and in mature β-cell function. Activation of transmembrane Notch receptors via their interaction with membrane-bound ligands, e.g. the Delta-like 4 leads to proteolytic steps that release the Notch intracellular domain (NICD) from the plasma membrane to the nucleus. The nuclear NICD enters into a transcriptional complex enabling activation of Notch target genes, which in turn negatively regulate expression of neurogenin-3 (Ngn3) transcription factor—a master regulator of pancreatic endocrine cell formation. In support of this model are studies demonstrating that disruption of Notch/ligand communication, or overexpression of Ngn3 in combination with other transcription factors, increases the output of hormone-producing cells in the pancreas and other organs.

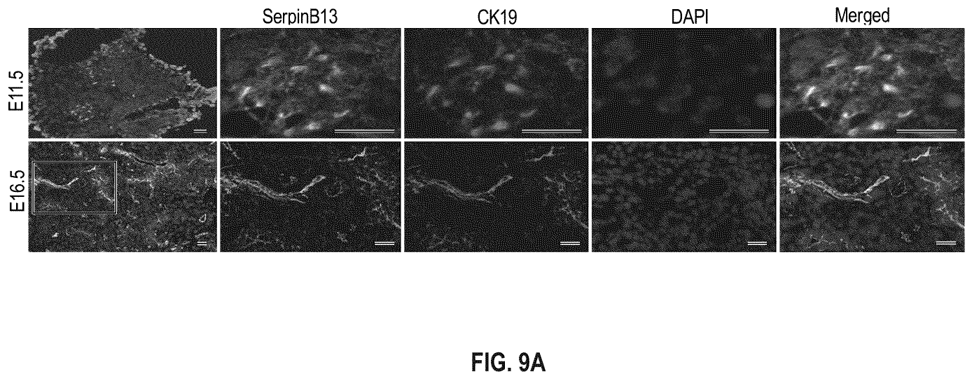

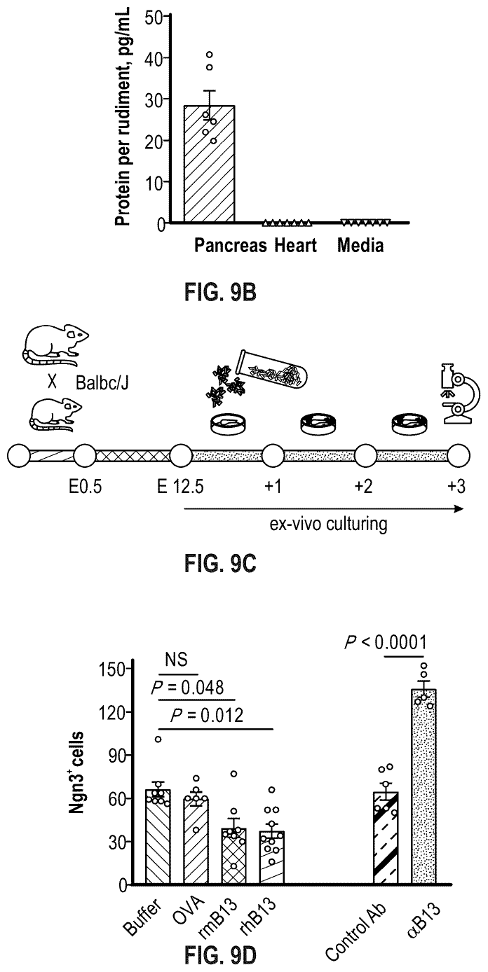

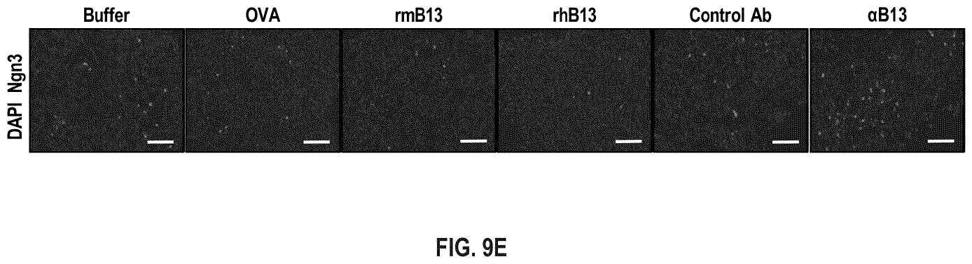

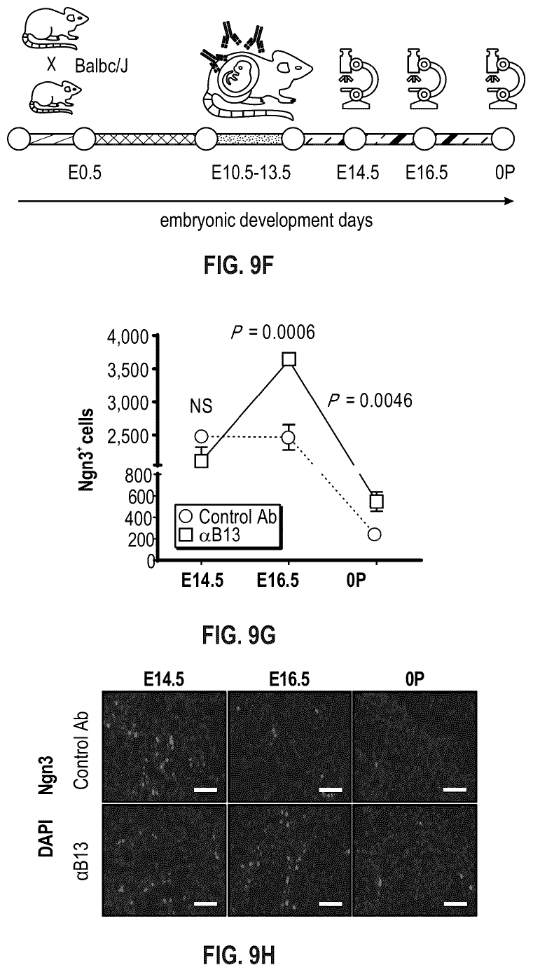

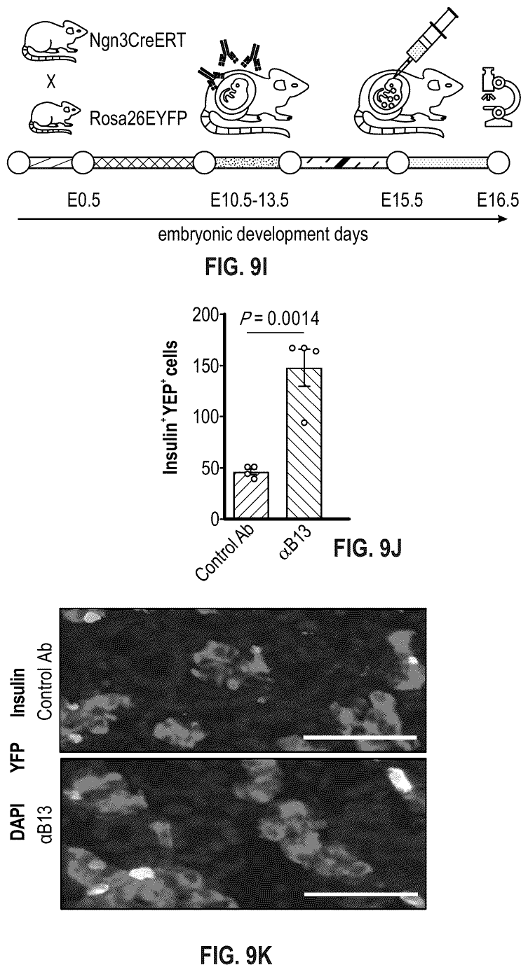

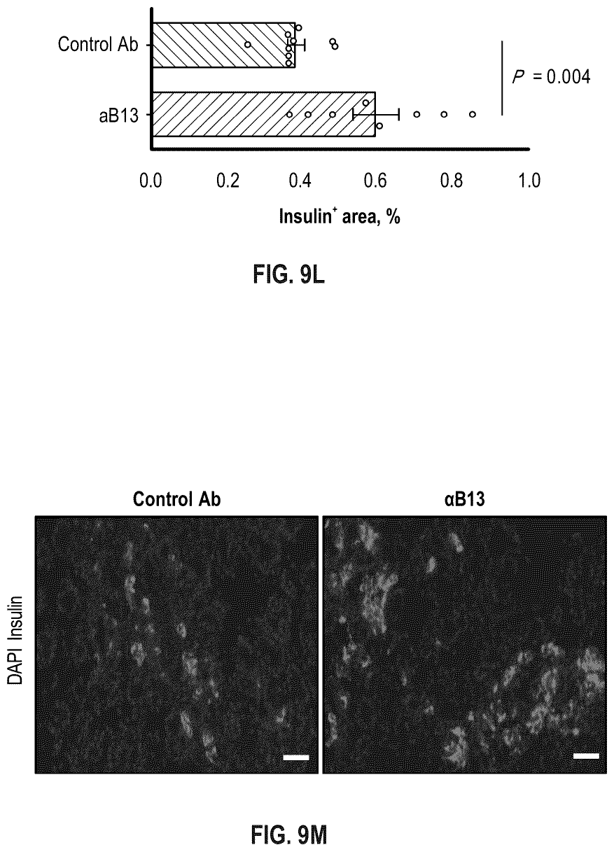

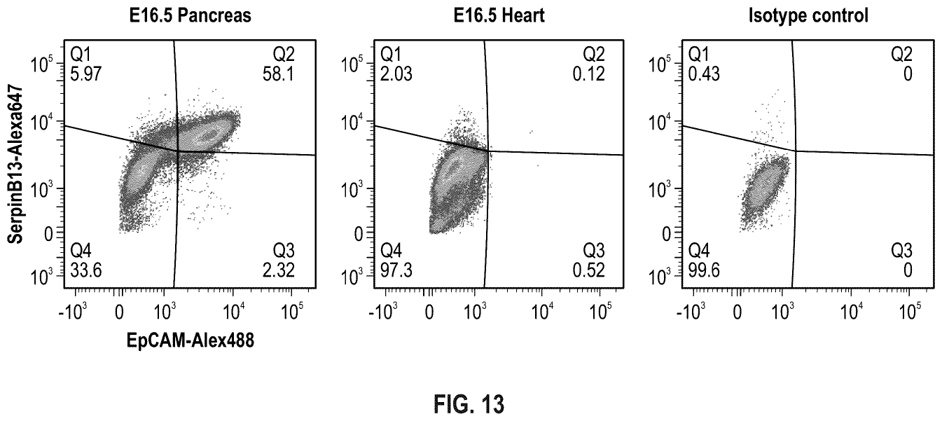

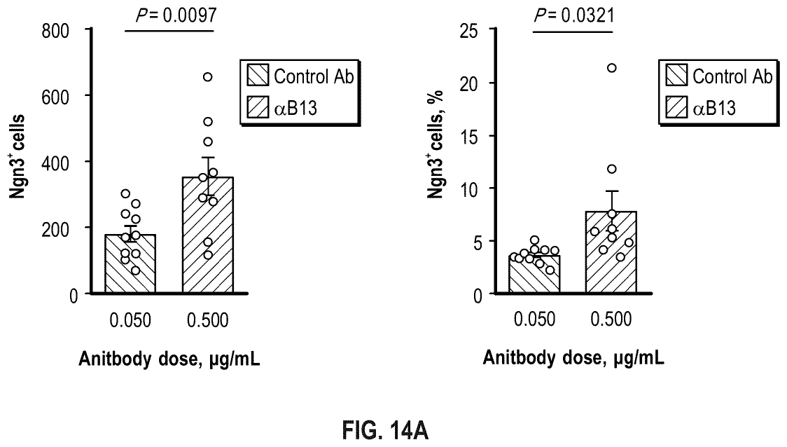

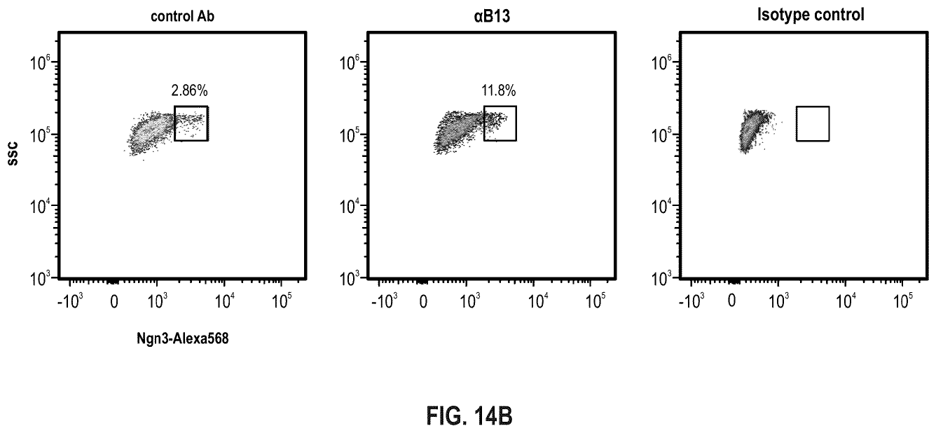

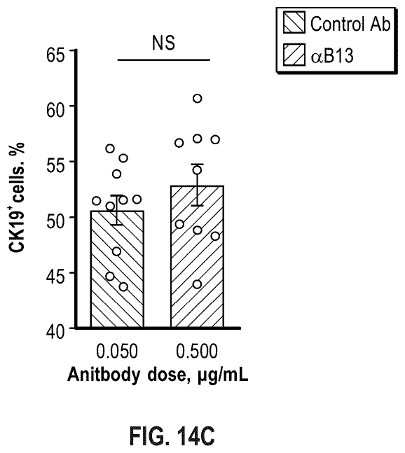

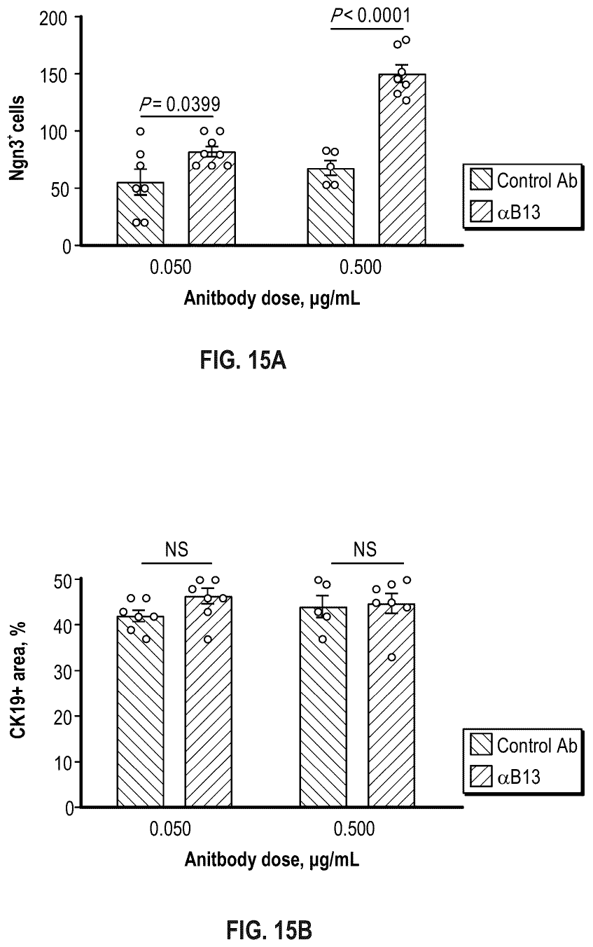

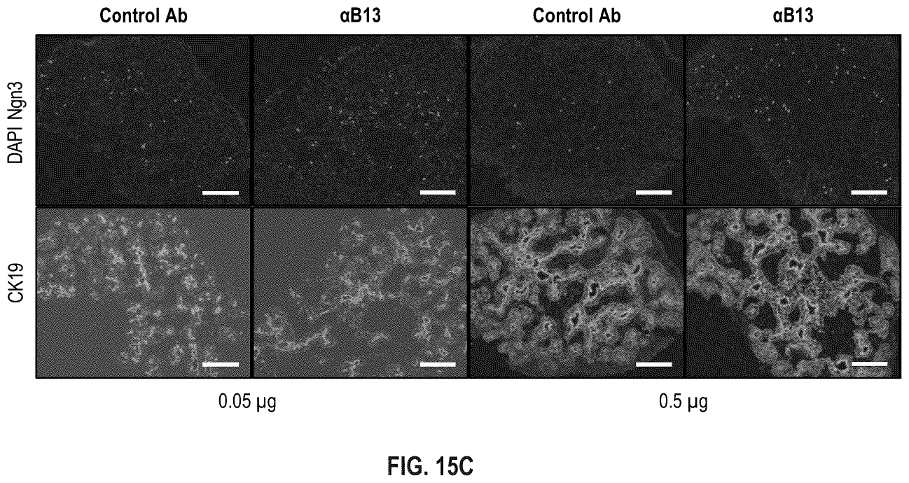

In the first attempt to gain insight into the potential role of serpinB13 in the development of the endocrine pancreas we examined its expression. We found this serpin to be confined to the cytokeratin-19⁺ (CK19⁺) epithelium as early as day E11.5 of gestation (FIG. 9A, FIG. 13). Furthermore, culturing embryonic pancreas explants at E12.5 for two days resulted in detectable serpinB13 level in the supernatant (FIG. 9B), suggesting that this molecule can be released to the extracellular milieu. When added to these in vitro cultures (FIG. 9C), both mouse and human recombinant serpinB13, but not chicken ovalbumin (a non-inhibitory structural homolog and founding member of the clade B serpin family), caused a significant drop in the number of Ngn3⁺ endocrine progenitor cells (FIGS. 9D and 9E). Conversely, inhibiting serpinB13 with a monoclonal antibody (mAb) originally developed in our laboratory, resulted in a significant increase in the number of Ngn3⁺ cells, compared with cultures treated with control Ab (FIGS. 9D and 9E, FIGS. 14A and 14B). This effect was observed over a wide mAb concentration range (FIGS. 15A and 15C) and was specific for Ngn3⁺ cells as the number of CK19⁺ epithelial cells did not change (FIG. 14C, FIGS. 15B and 15C).

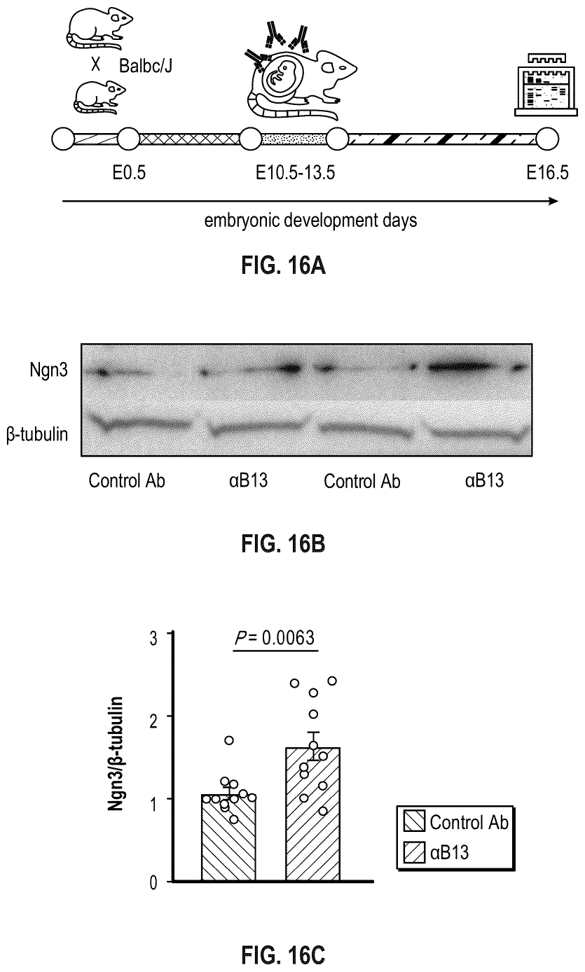

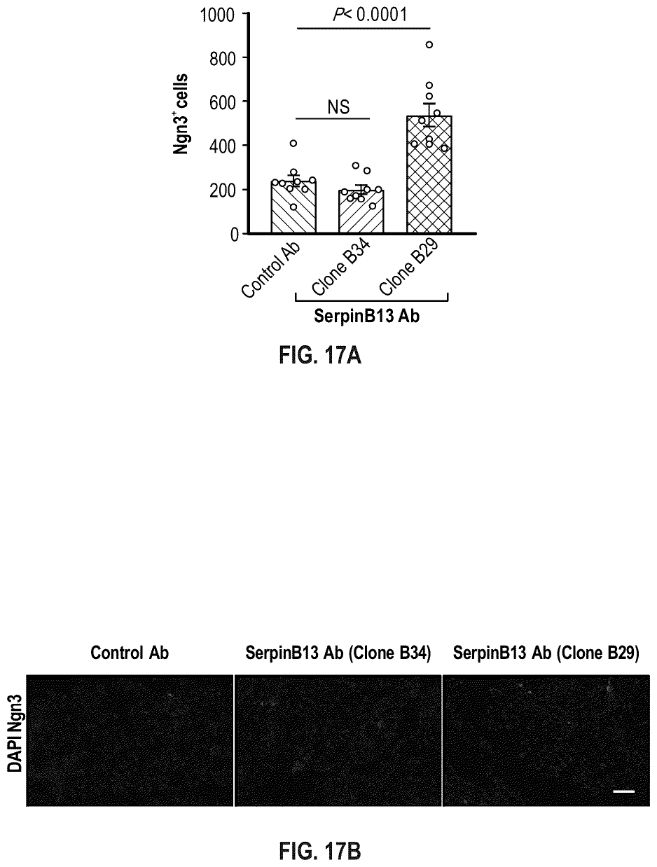

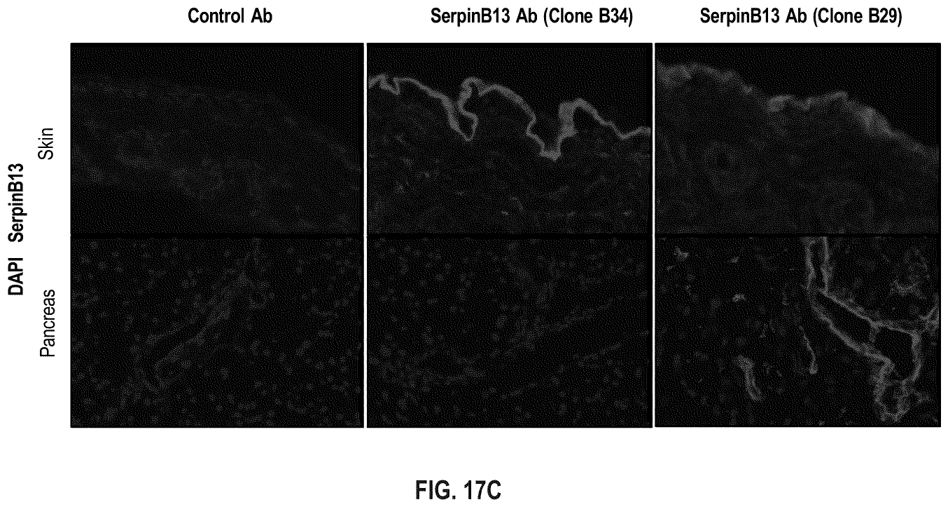

In addition to the above-mentioned increase in the number of pancreatic Ngn3⁺ cells in vitro, we also observed a significant expansion of the pancreatic Ngn3⁺ lineage in vivo. Mouse embryos isolated from pregnant mothers that had been injected for several days (E10.5 through E13.5) with anti-serpinB13 mAb (clone B29) (FIG. 9F) showed a robust increase in the number of Ngn3⁺ cells and protein levels at both embryonic day E16.5 and birth (OP) (FIGS. 9G and 9H, FIG. 16A-16C). Of note, clone B34 of anti-serpinB13 mAb, which fails to stain pancreatic epithelium (despite its ability to stain the epidermis, FIG. 17C), completely failed to have any effect on Ngn3⁺ cells (FIGS. 17A and 17B), thereby confirming the specificity of the changes we observed. Therefore, both in vitro and in vivo approaches to modify the extracellular level of serpinB13 impacted the development of pancreatic Ngn3⁺ cells.

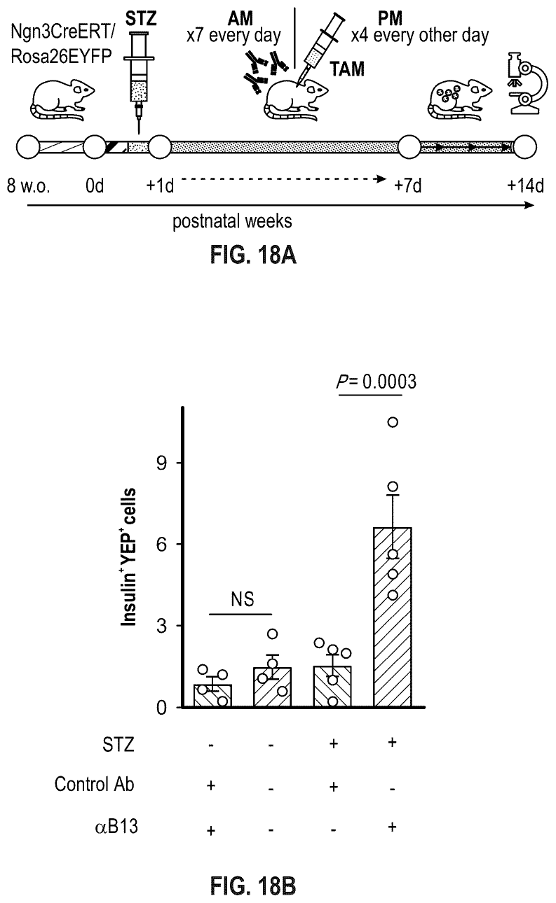

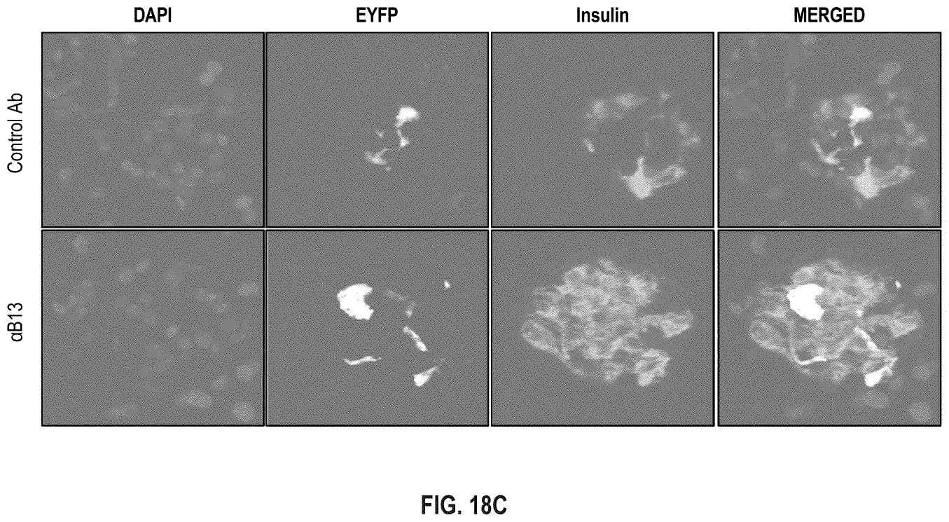

Importantly, genetic labelling of Ngn3⁺ cells following injection of anti-serpinB13 mAb during gestation (FIG. 9I) demonstrated an enhanced conversion of these cells to acquire insulin-positive status (FIGS. 9J and 9K). Consistent with this observation, the embryos that had been exposed in vivo to inhibition of serpinB13 with a mAb showed significantly higher fractions of pancreatic microscopic sections positively stained with anti-insulin antibody, compared with the embryos exposed to control Ab (FIGS. 9L and 9M). This enhanced transition from Ngn3 to insulin expression was not limited to the gestational period but was also observed during de novo formation of β-cells in the adult mice with streptozotocin (STZ)-induced pancreatic damage and inhibited serpinB13 (FIG. 18A-18C). Taken together, our observations thus far demonstrate that serpinB13 represses the generation of Ngn3⁺ cells, while its inhibition with a neutralizing antibody increases the Ngn3⁺ cell population and β-cell presence in the pancreas.

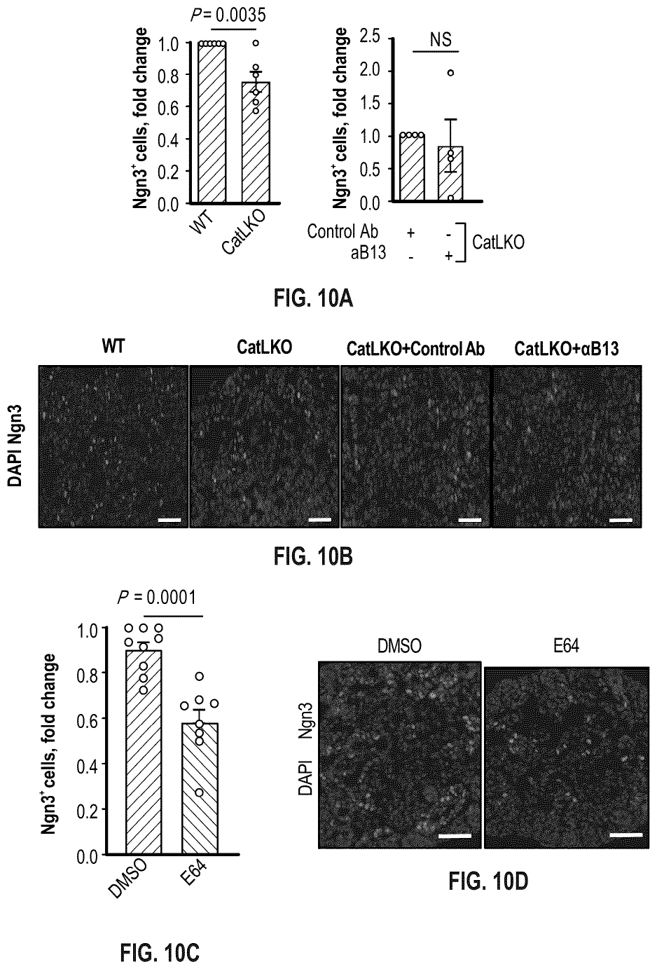

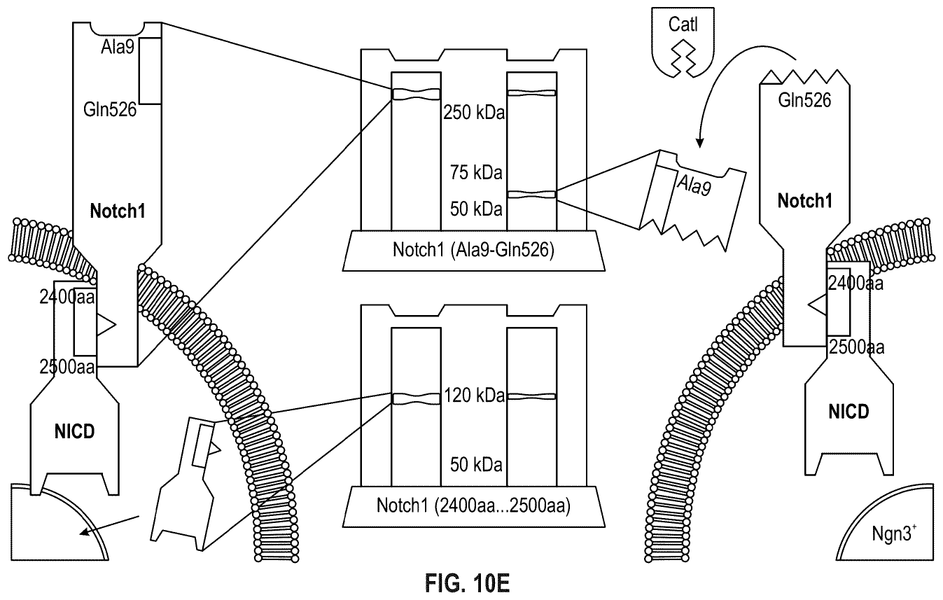

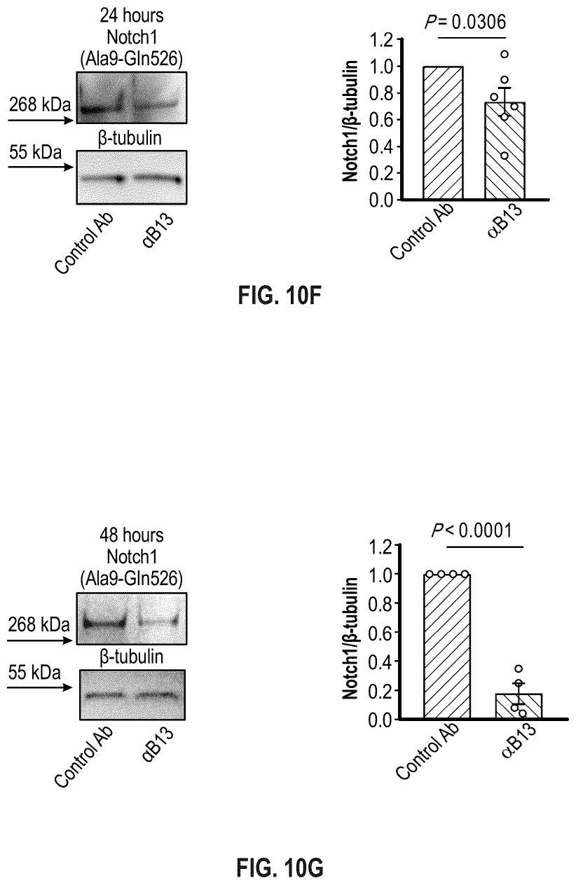

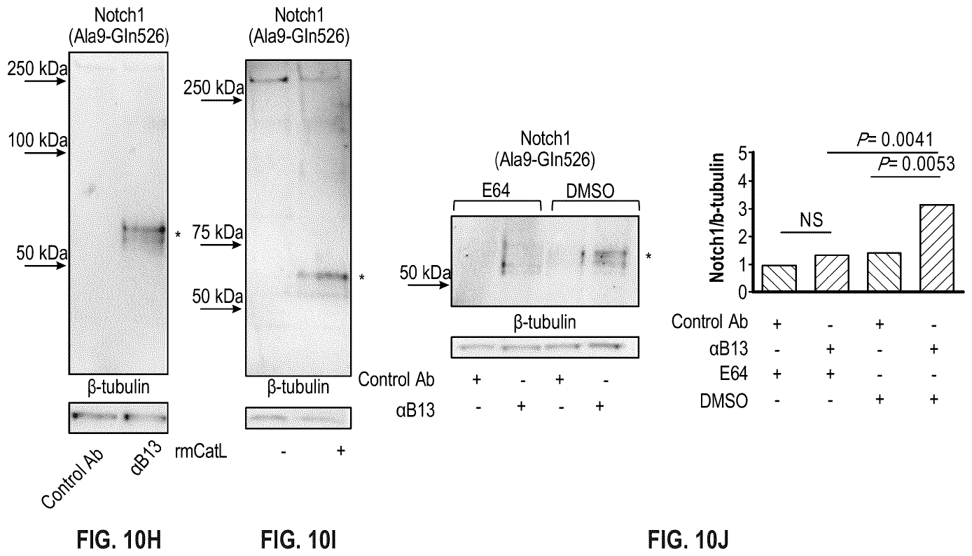

To better understand the role of the interplay between serpinB13 and CatL in the development of the endocrine pancreas we examined embryonic in vitro cultures for changes in Ngn3⁺ cells in CatL-deficient mice as well as after exposure to E64, which inhibits several proteases including CatL. In these settings, the size of the Ngn3⁺ cell population in the pancreas was significantly reduced (FIG. 10A—left and 10B, FIGS. 10C and 10D) and completely refractory to upregulation by anti-serpinB13 mAb (FIG. 10A—right and 10B). We continued to use anti-serpinB13 mAb (clone B29) as we previously showed that it blocked inhibitory properties of serpinB13 and allowed for the activity of its protease target, CatL to increase.

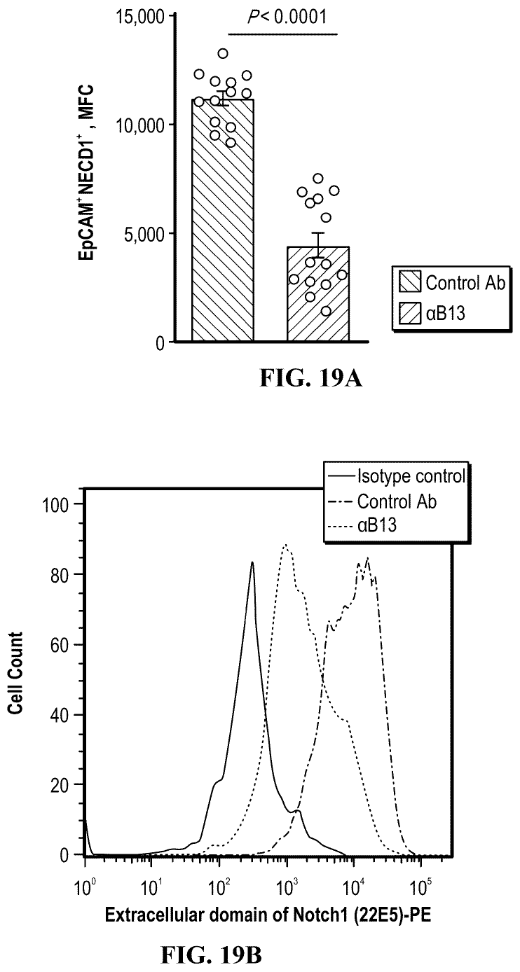

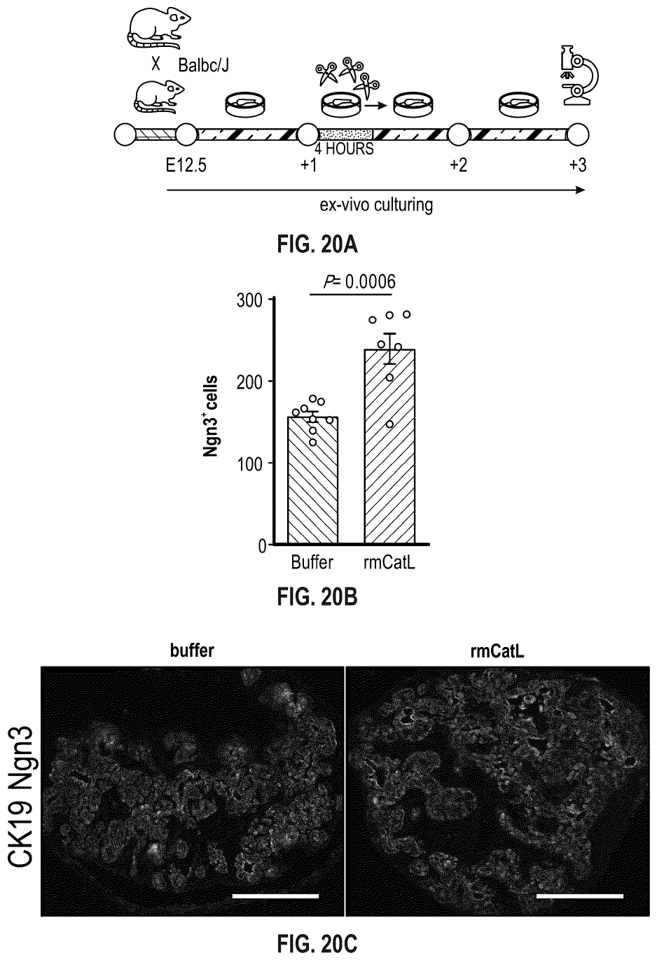

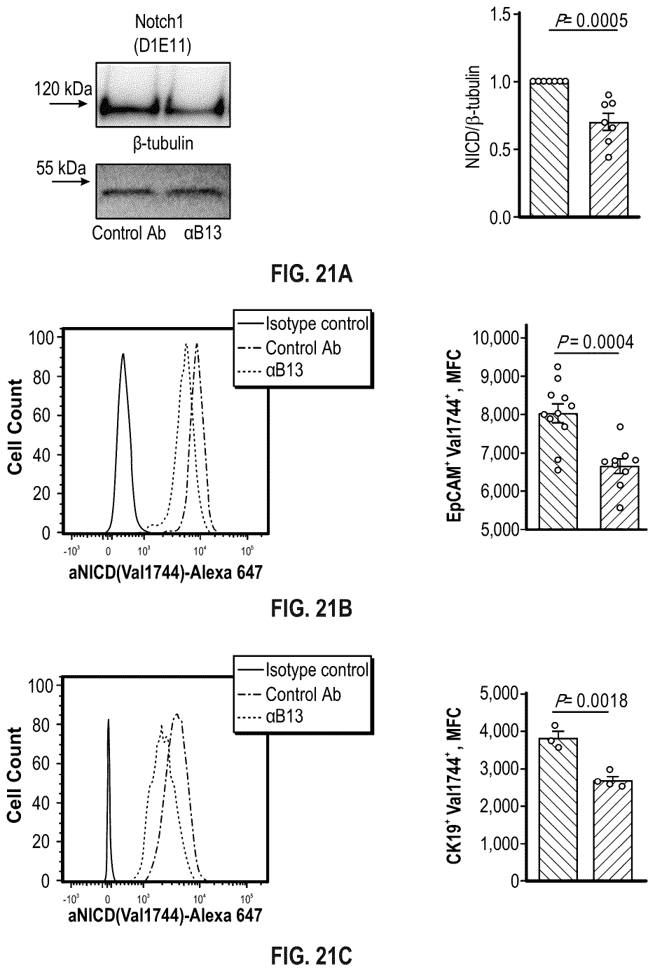

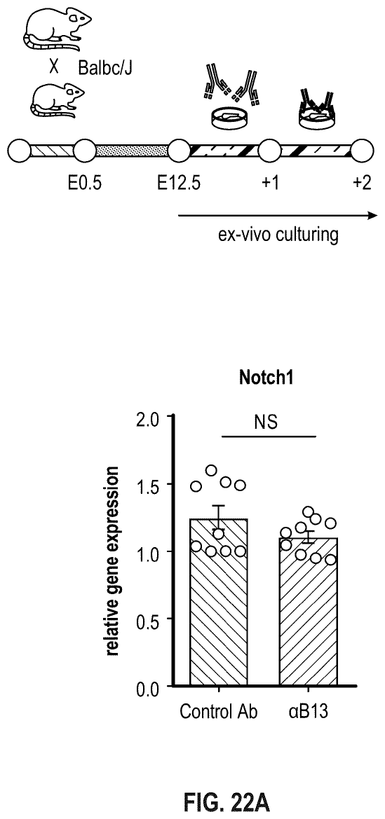

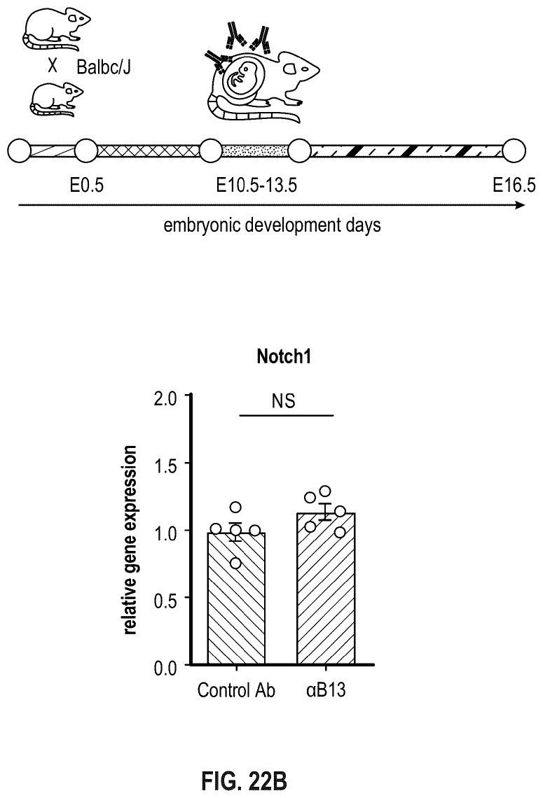

Since Ngn3 expression is negatively regulated by the Notch communication system, we wondered whether upregulation of the pool of Ngn3⁺ cells following inhibition of serpinB13 with mAb could stem from disruption of the Notch receptor expressed on the cell surface. Following experimental scheme for examination of the extracellular and intracellular domains of Notch1, depicted in FIG. 10E, we found that both in vivo and in vitro exposure to neutralizing mAb against serpinB13 resulted in a time-dependent reduced expression of the ectodomain of the Notch 1 receptor in embryonic pancreatic tissue (FIGS. 10F and 10G, FIG. 19A-19B) with concomitant induction of several ˜60 kD degradation fragments (FIG. 10H); which were almost completely prevented by the protease inhibitor, E64 (FIG. 10J). Similar results were obtained with recombinant CatL, which both cleaved the extracellular domain of Notch 1 (FIG. 10I) and induced additional Ngn3 cells (FIG. 20A-20C). The partial loss of extracellular Notch was followed by markedly reduced levels of active Notch intracellular domain (aNICD) (FIG. 21A-21C), indicating that Notch signaling was inhibited. Of note, the transcriptional level of Notch 1 was relatively intact (FIG. 22A-22B). These results suggest that serpinB13 helps to maintain the Notch receptor-mediated repression of pancreatic endocrine progenitors, and that perturbation of this serpinB13 functionality enables proteinase activity to dismantle Notch signaling, thereby allowing for more efficient development of Ngn3⁺ progenitors cells.

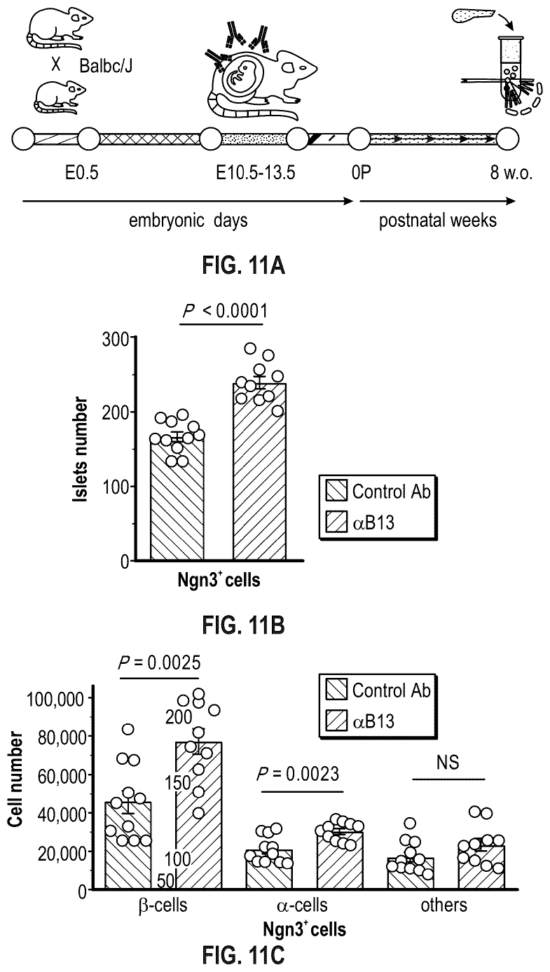

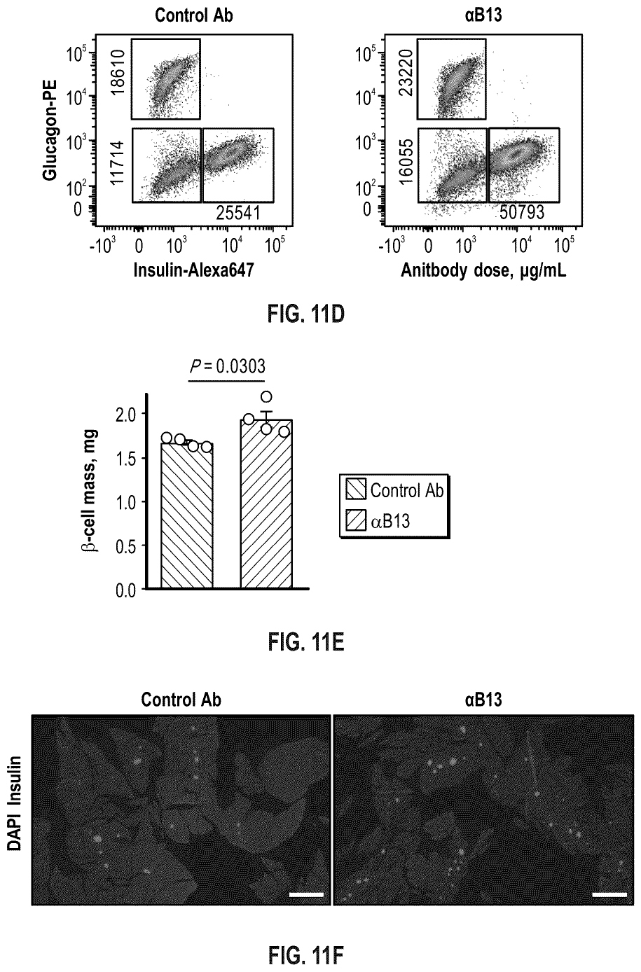

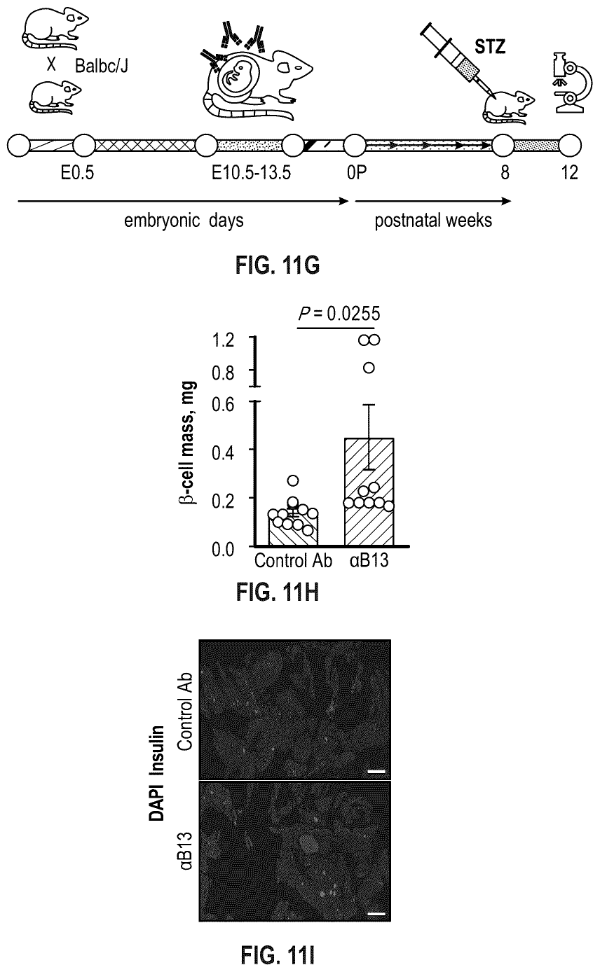

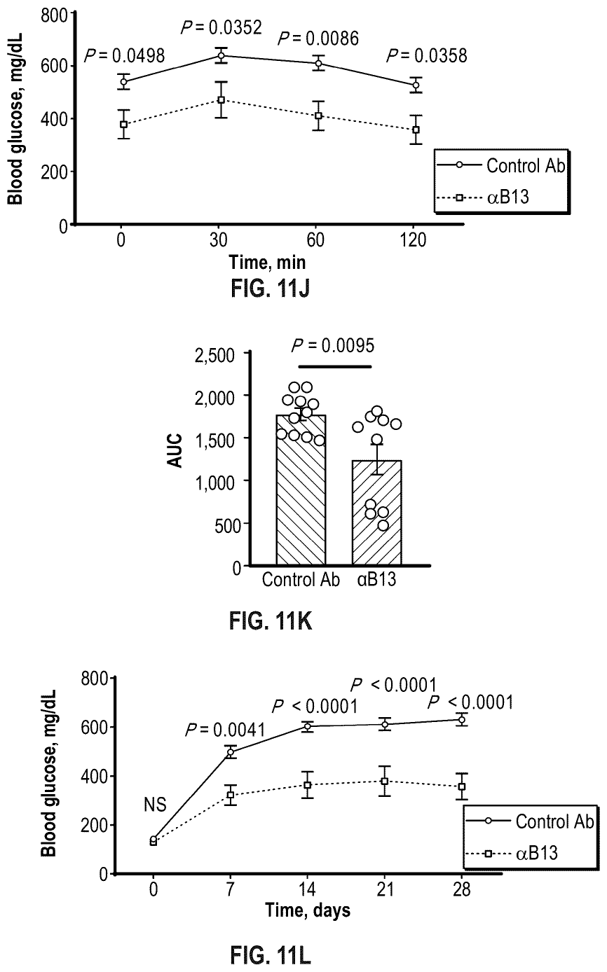

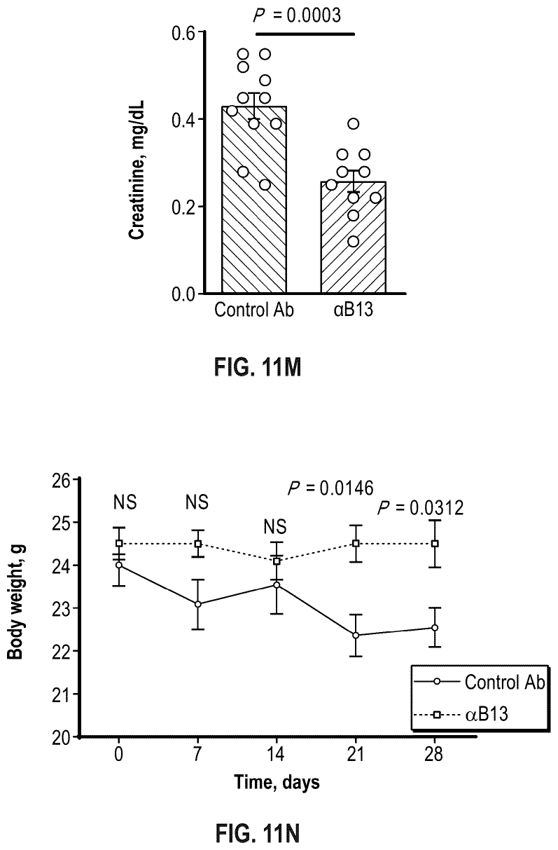

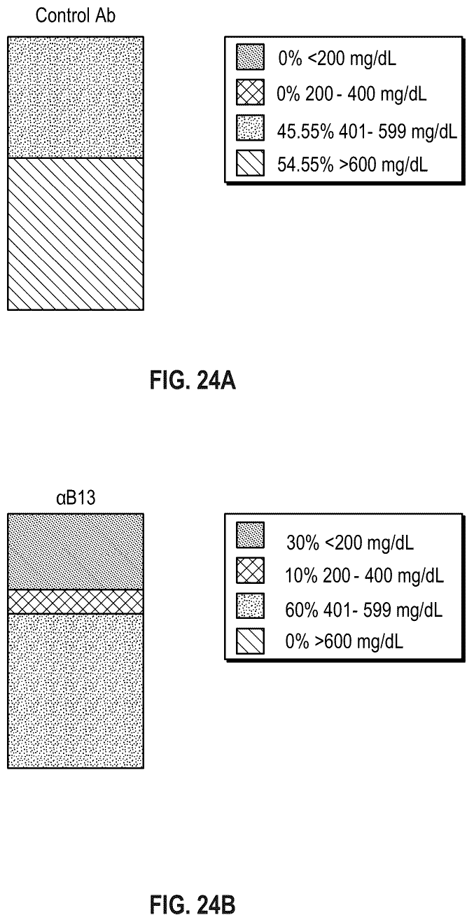

To assess the long-term impact of developmental changes induced by serpinB13 and its inhibition in the pancreas, we followed newborn mice for several months born from Balb/c mothers receiving anti-serpinB13 mAb during pregnancy (FIG. 11A). The prenatal inhibition of serpinB13 led to a significant increase in the number of pancreatic islets (FIG. 11B), total β-cell number (FIGS. 11C and 11D) and β-cell mass per animal (FIGS. 11E and 11F) in the 8-week old offspring (although the total pancreas and body weight at birth and in adulthood remained the same between the two groups, FIG. S123A-23B1). The α-cell population was also significantly increased, albeit to a lesser degree compared with the β-cells (FIG. 11C). Moreover, the postnatal increase in β-cell mass due to inhibition of serpinB13 during embryogenesis, was advantageous in the setting of diabetes induced with STZ injected in adulthood (FIG. 11G). It resulted in an over two-fold greater preserved residual β-cell mass (FIGS. 11H and 11I) and either completely protected against diabetes (FIG. 24A-24B), or ameliorated the severity of the disease (e.g., blood glucose control [FIGS. 11J, 11K and 11L] and serum creatinine as a measure of renal function [FIG. 11M] were improved while loss of body weight was prevented [FIG. 11N]) compared with the STZ-injected offspring of mothers that had received control antibody. Therefore, embryonal increase in the number of Ngn3⁺ endocrine progenitors by inhibition of serpinB13 offered a long-term benefit against abrupt loss of insulin-producing cells.

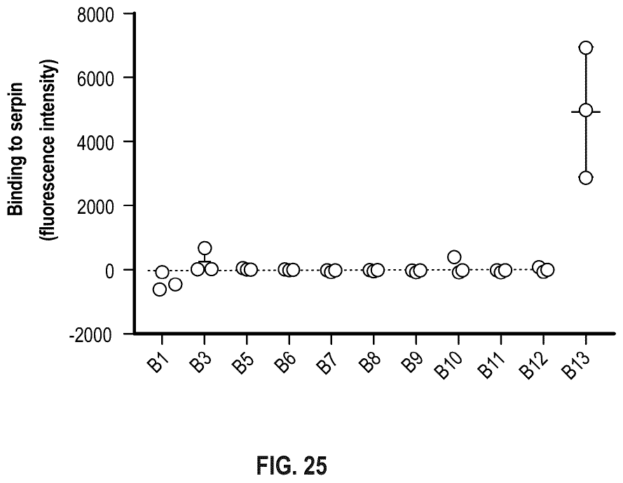

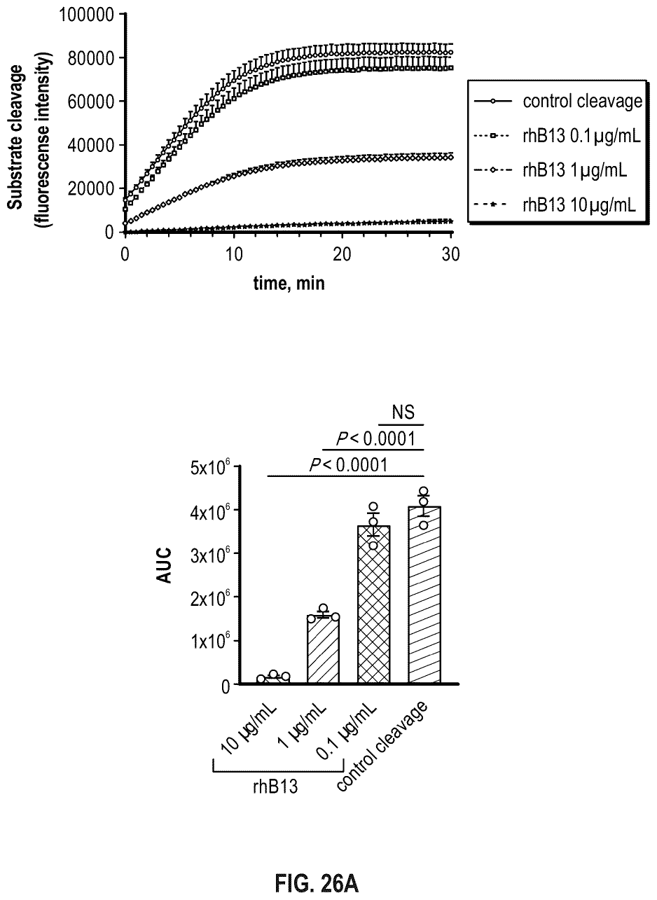

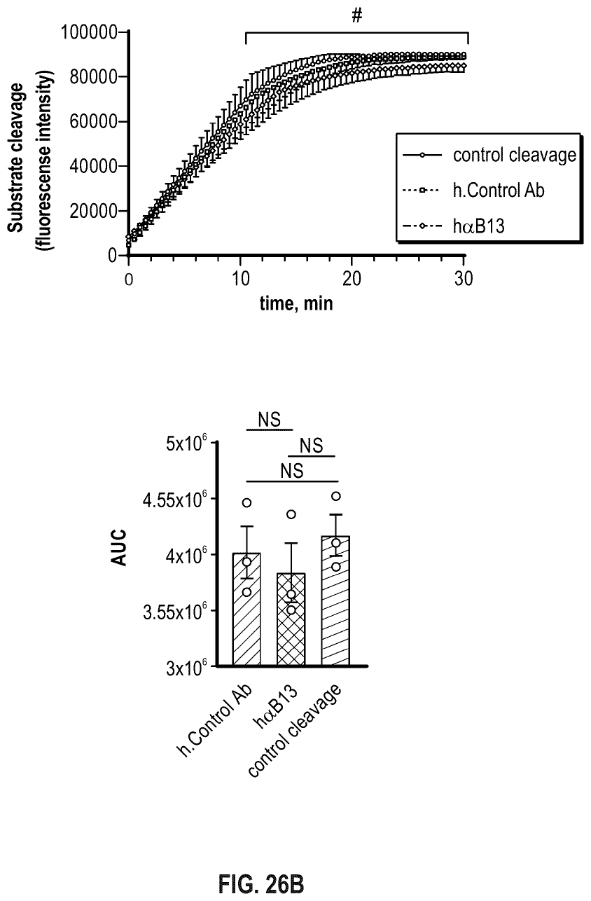

Previous studies in our laboratory revealed a novel autoantibody (AA) to serpinB13 and its association at baseline with higher residual fasting and stimulated C-peptide levels in humans with a recent-onset diagnosis of TID. Encouraged by this finding as well as our observations on serpinB13-mediated developmental changes in the pancreas and their impact on diabetes in mice we wondered whether serpinB13 AA influences the pre-diabetes period and progression to TID. To address this question, we measured baseline serpinB13 AA in subjects that were meticulously staged for risk of TID during enrollment in the Diabetes Prevention Trial for Type 1 Diabetes (DPT-1). The serological serpinB13 binding activity inversely correlated with the risk level for TID (FIG. 12A). Moreover, we found that the baseline serpinB13 AA was associated with a reduction in the overall incidence of diabetes (FIG. 12B) as well as longer diabetes-free survival in those who progressed to clinical disease (FIG. 12C). The phenotype in humans with serpinB13 AA could be explained by the potential functionality of this AA, which may mimic the function of the mouse mAb to serpinB13. Indeed, the dialyzed DPT-1 serum samples positive for serpinB13 AA significantly stimulated the development of Ngn3⁺ cells in in vitro cultures (FIGS. 12D and 12E). Similar results were obtained with a human recombinant antibody to serpinB13 (FIG. 25), which in a similar fashion to previously described by us mouse mAb to serpinB13 rescues CatL protease activity from inhibition by this serpin (FIG. 12F, FIG. 26A-26B). Specifically, when added to the dialyzed DPT-1 serum samples that originally were negative for serpinB13 AA, human antibody to serpinB13 induced additional Ngn3⁺ cells (FIGS. 12G and 12H). On the other hand, following experimental scheme do delete serpinB13AA from serum, depicted in FIG. 12I, serpinB13 positive serum samples failed to induce additional Ngn3⁺ cells after immunodepletion of this AA (FIGS. 12J and 12K). Taken together, serpinB13 AA may be actively involved in regulating the Notch pathway and diabetes prevention in a way that is similar to that described by us in our model using mouse anti-serpinB13 mAb.

This Example describes a novel function of a clade B serpin in the developing pancreas. We propose that the interplay between serpinB13 and its CatL proteinase target influences cell fate decision in differentiating pancreatic epithelium by limiting Notch signaling. Specifically, we argue that repressing the inhibitory function of serpinB13 allows for CatL-mediated partial impairment of Notch1 on the cell surface (FIG. 12L), the event that is known to promote the endocrine fate. The incomplete inhibition of Notch could be explained by a relatively limited presence of CatL and serpinB13 outside the cell. Although serpinB13 is primarily an intracellular molecule, we found that it can be detected in the extracellular space. This feature is not unique to serpinB13, as other members of the clade B serpin family have also been found extracellularly or expressed on the cell surface.

Influencing Notch signaling as part of a programming paradigm has a strong potential for therapy. We used diabetes as a model to demonstrate that our approach to inactivate serpinB13 with mAb modifies the Notch pathway in a way that offers a better clinical outcome. However, it is possible that therapeutic inhibition of serpinB13, or other clade B serpin members, could go beyond the prevention of diabetes and be applicable for therapeutic approaches to other pathological processes involving deregulated Notch signaling.

Finally, our examination of young humans at risk for TID revealed that natural autoantibodies to serpinB13 offer a higher level of protection against the clinical onset of diabetes. This positive outcome may be attributed to the enhanced yield of Ngn3⁺ endocrine progenitors, which according to some authors can arise after birth under conditions of cellular injury or inflammatory cytokine stress in the pancreatic exocrine ductal cells or their vicinity. However, based on our previous studies we cannot exclude that serpinB13 AA in TID subjects also stimulate CatL-mediated cleavage of key cell-surface receptors, including those expressed in lymphocytes, e.g., CD4 in T cells and CD19 in B cells. Hence, both islet adaptive changes by newly generated Ngn3⁺ endocrine progenitor cells and the impediment of autoimmune inflammation in this tissue compartment may account for the protective impact of anti-serpinB13 activity in humans with TID.

### Materials and Methods

Experimental animals. Balb/cJ mice (stock No: 000651), C57BL/6J (stock No: 000664), Ngn3Cre:Tg (Neurog3-cre/Esr1*) 1 Dam/J (stock No: 008119) and Rosa26EYFP:B6.129X1-Gt (ROSA) 26Sortml (EYFP) Cos/J (stock No: 006148) were from the Jackson Laboratory (Bar Harbor, ME, USA). The CatL-deficient NOD mice (stock No. 008352) were back-crossed to the Balb/c background for at least 20 generations. The Institutional Animal Care and Use Committee approved all mice experiments.

SerpinB13 mAbs and other antibodies used in functional studies. The mouse mAb to serpinB13, clone B29, has been described previously (J. Czyzyk, O. Henegariu, P. Preston-Hurlburt, R. Baldzizhar, C. Fedorchuk, E. Esplugues, K. Bottomly, F. K. Gorus, K. Herold, R. A. Flavell, Enhanced anti-serpin antibody activity inhibits autoimmune inflammation in type 1 diabetes. J. Immunol. 188, 6319-6327 (2012)). The mouse mAb to serpin B13, clone B34, has also been previously developed in our laboratory but not published before. The epitope specificity of B34 is distinct from that of B29 and corresponds to the following amino acid sequence of mouse serpinB13—SEEEEIEKREEIHHQLQMLL (SEQ ID NO: 86).

The recombinant human antibodies to serpinB13 were developed from a human Fab library, constituting sequences derived from the antibody repertoire of approximately 120 individuals, with a diversity/complexity of approximately 1×10¹¹ clones (ProMab Biotechnologies, Richmond, CA, USA). Briefly, a scFv surface-display library was subjected to multiple rounds of screening by panning and flow cytometry against human serpinB13, following which the positive clones were isolated, re-tested for their binding to serpinB13, and selected for detailed testing. The clones were then selected for sequencing of the CDR region of heavy and light immunoglobulin chains. The heavy and light chain regions were amplified from cDNA by a two-step, nested PCR reaction using advantage 3 cDNA polymerase and primer mixes specific for germline families (VBASE database). Expression plasmids encoding sequences of full-length heavy and light-chain were used to produce recombinant antibodies in HEK203 cell expression system. The sequences for the heavy and light chains for the antibodies are provided in Table 2 below.

TABLE 2
Table of Sequences

| SEQ | | |
|---|---|---|
| ID | | |
| NO | Description | Sequence |
| 30 | Clone1-H(m1)- | CAGGTGCAGCTGCAGGAGTCCGGGGGAGGCGTGGTCCAGCCTGGGA |
| | DNA | GGTCCCTGAGACTCTCCTGTGCAGCCTCTGGATTCACCTTCAGTAG |
| | | CTATGGCATGCACTGGGTCCGCCAGGCTCCAGGCAAGGGGCTGGAG |
| | | TGGGTGGCAGTTATATCATATGATGGAAGTAATAAATACTATGCAG |
| | | ACTCCGTGAAGGGCCGATTCACCATCTCCAGAGACAATTCCAAGAA |
| | | CACGCTGTATCTGCAAATGAACAGCCTGAGAGCCGAGGACACGGCT |
| | | GTGTATTACTGTGCGAGAGATCTCGGCGCCGTTATAGCAGTGGCTG |
| | | GTACTTCCACCCCGTACAACTGGTTCGACCCCTGGGGCCAGGGAAC |
| | | CCTGGTCACCGTCTCAAGCGCCTCCACCAAGGGCCCATCGGTCTTC |
| | | CCCCTGGCACCCTCCTCCAAGAGCACCTCTGGGGGCACAGCGGCCC |
| | | TGGGCTGCCTGGTCAAGGACTACTTCCCCGAACCGGTGACGGTGTC |
| | | GTGGAACTCAGGCGCCCTGACCAGCGGCGTCCACACCTTCCCGGCT |
| | | GTCCTACAGTCCTCAGGACTCTACTCCCTCAGCAGCGTAGTGACCG |
| | | TGCCCTCCAGCAGCTTGGGCACCCAGACCTACATCTGCAACGTGAA |
| | | TCACAAGCCCAGCAACACCAAGGTGGACAAGAAAGTTGAGCCCAAA |
| | | TCTTGTGACAAAACTCACACATGCCCACCGTGCCCAGCACCTGAAC |
| | | TCCTGGGGGGACCGTCAGTCTTCCTCTTCCCCCCAAAACCCAAGGA |
| | | CACCCTCATGATCTCCCGGACCCCTGAGGTCACATGCGTGGTGGTG |
| | | GACGTGAGCCACGAAGACCCTGAGGTCAAGTTCAACTGGTACGTGG |
| | | ACGGCGTGGAGGTGCATAATGCCAAGACAAAGCCGCGGGAGGAGCA |
| | | GTACAACAGCACGTACCGTGTGGTCAGCGTCCTCACCGTCCTGCAC |
| | | CAGGACTGGCTGAATGGCAAGGAGTACAAGTGCAAGGTCTCCAACA |
| | | AAGCCCTCCCAGCCCCCATCGAGAAAACCATCTCCAAAGCCAAAGG |
| | | GCAGCCCCGAGAACCACAGGTGTACACCCTGCCCCCATCCCGGGAT |
| | | GAGCTGACCAAGAACCAGGTCAGCCTGACCTGCCTGGTCAAAGGCT |
| | | TCTATCCCAGCGACATCGCCGTGGAGTGGGAGAGCAATGGGCAGCC |
| | | GGAGAACAACTACAAGACCACGCCTCCCGTGCTGGACTCCGACGGC |
| | | TCCTTCTTCCTCTACAGCAAGCTCACCGTGGACAAGAGCAGGTGGC |
| | | AGCAGGGGAACGTCTTCTCATGCTCCGTGATGCATGAGGCTCTGCA |
| | | CAACCACTACACGCAGAAGAGCCTCTCCCTGTCTCCGGGTAAATGA |
| 31 | Clone1-H(m1)- | QVQLQESGGGVVQPGRSLRLSCAASGFTFSSYGMHWVRQAPGKGLE |
| | protein | WVAVISYDGSNKYYADSVKGRFTISRDNSKNTLYLQMNSLRAEDTA |
| | | VYYCARDLGAVIAVAGTSTPYNWFDPWGQGTLVTVSSASTKGPSVF |
| | | PLAPSSKSTSGGTAALGCLVKDYFPEPVTVSWNSGALTSGVHTFPA |
| | | VLQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDG |
| | | SFFLYSKLTVDKSRWQQGNVFSCSVMHEALHNHYTQKSLSLSPGK |
| 32 | vector DNA | ATGGAGACAGACACACTCCTGCTATGGGTACTGCTGCTCTGGGTTC |
| | | CAGGTTCCACTggcgccggatca |
| 33 | Clone1-H(m1)- | ATGGAGACAGACACACTCCTGCTATGGGTACTGCTGCTCTGGGTTC |
| | Recombinant | CAGGTTCCACTggcgccggatca |
| | DNA Sequence | CAGGTGCAGCTGCAGGAGTCCGGGGGAGGCGTGGTCCAGCCTGGGA |
| | (vector: pYD5- | GGTCCCTGAGACTCTCCTGTGCAGCCTCTGGATTCACCTTCAGTAG |
| | hFC(Amp)) | CTATGGCATGCACTGGGTCCGCCAGGCTCCAGGCAAGGGGCTGGAG |
| | | TGGGTGGCAGTTATATCATATGATGGAAGTAATAAATACTATGCAG |
| | | ACTCCGTGAAGGGCCGATTCACCATCTCCAGAGACAATTCCAAGAA |
| | | CACGCTGTATCTGCAAATGAACAGCCTGAGAGCCGAGGACACGGCT |
| | | GTGTATTACTGTGCGAGAGATCTCGGCGCCGTTATAGCAGTGGCTG |
| | | GTACTTCCACCCCGTACAACTGGTTCGACCCCTGGGGCCAGGGAAC |
| | | CCTGGTCACCGTCTCAAGCGCCTCCACCAAGGGCCCATCGGTCTTC |
| | | CCCCTGGCACCCTCCTCCAAGAGCACCTCTGGGGGCACAGCGGCCC |
| | | TGGGCTGCCTGGTCAAGGACTACTTCCCCGAACCGGTGACGGTGTC |
| | | GTGGAACTCAGGCGCCCTGACCAGCGGCGTCCACACCTTCCCGGCT |
| | | GTCCTACAGTCCTCAGGACTCTACTCCCTCAGCAGCGTAGTGACCG |
| | | TGCCCTCCAGCAGCTTGGGCACCCAGACCTACATCTGCAACGTGAA |
| | | TCACAAGCCCAGCAACACCAAGGTGGACAAGAAAGTTGAGCCCAAA |
| | | TCTTGTGACAAAACTCACACATGCCCACCGTGCCCAGCACCTGAAC |
| | | TCCTGGGGGGACCGTCAGTCTTCCTCTTCCCCCCAAAACCCAAGGA |
| | | CACCCTCATGATCTCCCGGACCCCTGAGGTCACATGCGTGGTGGTG |
| | | GACGTGAGCCACGAAGACCCTGAGGTCAAGTTCAACTGGTACGTGG |
| | | ACGGCGTGGAGGTGCATAATGCCAAGACAAAGCCGCGGGAGGAGCA |
| | | GTACAACAGCACGTACCGTGTGGTCAGCGTCCTCACCGTCCTGCAC |
| | | CAGGACTGGCTGAATGGCAAGGAGTACAAGTGCAAGGTCTCCAACA |
| | | AAGCCCTCCCAGCCCCCATCGAGAAAACCATCTCCAAAGCCAAAGG |
| | | GCAGCCCCGAGAACCACAGGTGTACACCCTGCCCCCATCCCGGGAT |
| | | GAGCTGACCAAGAACCAGGICAGCCIGACCIGCCIGGTCAAAGGCT |
| | | TCTATCCCAGCGACATCGCCGTGGAGTGGGAGAGCAATGGGCAGCC |
| | | GGAGAACAACTACAAGACCACGCCTCCCGTGCTGGACTCCGACGGC |
| | | TCCTTCTTCCTCTACAGCAAGCTCACCGTGGACAAGAGCAGGTGGC |
| | | AGCAGGGGAACGTCTTCTCATGCTCCGTGATGCATGAGGCTCTGCA |
| | | CAACCACTACACGCAGAAGAGCCICICCCTGTCTCCGGGTAAATGA |
| 34 | vector protein | METDTLLLWVLLLWVPGSTGAGS |
| 35 | Clone1-H(m1) - | METDTLLLWVLLLWVPGSTGAGS |
| | Recombinant | QVQLQESGGGVVQPGRSLRLSCAASGFTFSSYGMHWVRQAPGKGLE |
| | protein Sequence | WVAVISYDGSNKYYADSVKGRFTISRDNSKNTLYLQMNSLRAEDTA |
| | (vector: pYD5- | VYYCARDLGAVIAVAGTSTPYNWFDPWGQGTLVTVSSASTKGPSVF |
| | hFC(Amp)) | PLAPSSKSTSGGTAALGCLVKDYFPEPVTVSWNSGALTSGVHTFPA |
| | | VLQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDG |
| | | SFFLYSKLTVDKSRWQQGNVFSCSVMHEALHNHYTQKSLSLSPGK |
| 36 | Clone1-L(m1) | GATGTTGTGATGACTCAGTCTCCACTCTCCCTGCCCGTCACCCCTG |
| | DNA | GAGAGCCGGCCTCCATCTCCTGCAGGTCTCGTCAGAGCCTCCTGCA |
| | | TAGCAATGGACACAACTATTTGGGTTGGTACCTGCAGAAGCCAGGG |
| | | CAGTCTCCACAGCTCCTGATCTATCTGGCTTCTATTCGGGCCTCCG |
| | | GGATCCCTGACAGGTTCAGTGGCAGTGGATCAGGCACAGATTTTAC |
| | | ACTGAAAATCAGCAGAGTGGAGGCTGAGGATGTTGGCGTTTATTAC |
| | | TGCATGCAAGCTCTACAAACACCCTACACTTTTGGCCAGGGGACCA |
| | | AGCTGGAGATCAAGCGAACTGTGGCTGCACCATCTGTCTTCATCTT |
| | | CCCGCCATCTGATGAGCAGTTGAAATCTGGAACTGCCTCTGTTGIG |
| | | TGCCTGCTGAATAACTTCTATCCCAGAGAGGCCAAAGTACAGTGGA |
| | | AGGTGGATAACGCCCTCCAATCGGGTAACTCCCAGGAGAGTGTCAC |
| | | AGAGCAGGACAGCAAGGACAGCACCTACAGCCTCAGCAGCACCCTG |
| | | ACGCTGAGCAAAGCAGACTACGAGAAACACAAAGTCTACGCCTGCG |
| | | AAGTCACCCATCAGGGCCTGAGCTCGCCCGTCACAAAGAGCTTCAA |
| | | CAGGGGAGAGTGTTAG |
| 37 | Clone1-L(m1) | DVVMTQSPLSLPVTPGEPASISCRSRQSLLHSNGHNYLGWYLQKPG |
| | protein | QSPQLLIYLASIRASGIPDRFSGSGSGTDFTLKISRVEAEDVGVYY |
| | | CMQALQTPYTFGQGTKLEIKRTVAAPSVFIFPPSDEQLKSGTASVV |
| | | CLLNNFYPREAKVQWKVDNALQSGNSQESVTEQDSKDSTYSLSSTL |
| | | TLSKADYEKHKVYACEVTHQGLSSPVTKSFNRGEC |
| 38 | Clone1-L(m1) - | ATGGAGACAGACACACTCCTGCTATGGGTACTGCTGCTCTGGGTTC |
| | Recombinant | CAGGTTCCACTggcgccggatca |
| | DNA Sequence | GATGTTGTGATGACTCAGTCTCCACTCTCCCTGCCCGTCACCCCTG |
| | (Vector: pYD5- | GAGAGCCGGCCTCCATCTCCTGCAGGTCTCGTCAGAGCCTCCTGCA |
| | hFC(Amp)) | TAGCAATGGACACAACTATTTGGGTTGGTACCTGCAGAAGCCAGGG |
| | | CAGTCTCCACAGCTCCTGATCTATCTGGCTTCTATTCGGGCCTCCG |
| | | GGATCCCTGACAGGTTCAGTGGCAGTGGATCAGGCACAGATTTTAC |
| | | ACTGAAAATCAGCAGAGTGGAGGCTGAGGATGTTGGCGTTTATTAC |
| | | TGCATGCAAGCTCTACAAACACCCTACACTTTTGGCCAGGGGACCA |
| | | AGCTGGAGATCAAGCGAACTGTGGCTGCACCATCTGTCTTCATCTT |
| | | CCCGCCATCTGATGAGCAGTTGAAATCTGGAACTGCCTCTGTTGTG |
| | | TGCCTGCTGAATAACTTCTATCCCAGAGAGGCCAAAGTACAGTGGA |
| | | AGGTGGATAACGCCCTCCAATCGGGTAACTCCCAGGAGAGTGTCAC |
| | | AGAGCAGGACAGCAAGGACAGCACCTACAGCCTCAGCAGCACCCTG |
| | | ACGCTGAGCAAAGCAGACTACGAGAAACACAAAGTCTACGCCTGCG |
| | | AAGTCACCCATCAGGGCCTGAGCTCGCCCGTCACAAAGAGCTTCAA |
| | | CAGGGGAGAGTGTTAG |
| 39 | Clone1-L(m1) - | METDTLLLWVLLLWVPGSTGAGS |
| | Recombinant | DVVMTQSPLSLPVTPGEPASISCRSRQSLLHSNGHNYLGWYLQKPG |
| | Protein Sequence | QSPQLLIYLASIRASGIPDRFSGSGSGTDFTLKISRVEAEDVGVYY |
| | (Vector: pYD5- | CMQALQTPYTFGQGTKLEIKRTVAAPSVFIFPPSDEQLKSGTASVV |
| | hFC(Amp)) | CLLNNFYPREAKVQWKVDNALQSGNSQESVTEQDSKDSTYSLSSTL |
| | | TLSKADYEKHKVYACEVTHQGLSSPVTKSFNRGEC |
| 40 | CLONE2-H(m1) | CAGATGCAGCTGGTGCAGTCGGGGGGAGGTGTGGTACGGCCTGGGG |
| | DNA Sequence | GGTCCCTGAGACTCTCCTGTGCAGCCTCTGGATTCACCTTTGATGA |
| | | TTATGGCATGAGCTGGGTCCGCCAAGCTCCAGGGAAGGGGCTGGAG |
| | | GGGTCTCTGGTATTAATTGGAATGGTGGTAGCACAGGTTATGCAG |
| | | ACTCTGTGAAGGGCCGATTCACCATCTCCAGAGACAACGCCAAGAA |
| | | CTCCCTGTATCTGCAAATGAACAGTCTGAGAGCCGAGGACACGGCC |
| | | TTGTATTACTGTGCGAGAGAAAGCTCGATGACTACAGTAACTACGT |
| | | ATCTCCTACGGGAAGTAGGGGTAGGGTTGGACTTTGACTACTGGGG |
| | | CCAGGGCACCCTGGTCACCGTCTCAAGCGCCTCCACCAAGGGCCCA |
| | | TCGGTCTTCCCCCTGGCACCCTCCTCCAAGAGCACCTCTGGGGGCA |
| | | CAGCGGCCCTGGGCTGCCTGGTCAAGGACTACTTCCCCGAACCGGT |
| | | GACGGTGTCGTGGAACTCAGGCGCCCTGACCAGCGGCGTCCACACC |
| | | TTCCCGGCTGTCCTACAGTCCTCAGGACTCTACTCCCTCAGCAGCG |
| | | TAGTGACCGTGCCCTCCAGCAGCTTGGGCACCCAGACCTACATCTG |
| | | CAACGTGAATCACAAGCCCAGCAACACCAAGGTGGACAAGAAAGTT |
| | | GAGCCCAAATCTTGTGACAAAACTCACACATGCCCACCGTGCCCAG |
| | | CACCTGAACTCCTGGGGGGACCGTCAGTCTTCCICTTCCCCCCAAA |
| | | ACCCAAGGACACCCTCATGATCTCCCGGACCCCTGAGGTCACATGC |
| | | GTGGTGGTGGACGTGAGCCACGAAGACCCTGAGGTCAAGTTCAACT |
| | | GGTACGTGGACGGCGTGGAGGTGCATAATGCCAAGACAAAGCCGCG |
| | | GGAGGAGCAGTACAACAGCACGTACCGTGTGGTCAGCGTCCTCACC |
| | | GTCCTGCACCAGGACTGGCTGAATGGCAAGGAGTACAAGTGCAAGG |
| | | TCTCCAACAAAGCCCTCCCAGCCCCCATCGAGAAAACCATCTCCAA |
| | | AGCCAAAGGGCAGCCCCGAGAACCACAGGTGTACACCCTGCCCCCA |
| | | TCCCGGGATGAGCTGACCAAGAACCAGGTCAGCCTGACCTGCCTGG |
| | | TCAAAGGCTTCTATCCCAGCGACATCGCCGTGGAGTGGGAGAGCAA |
| | | TGGGCAGCCGGAGAACAACTACAAGACCACGCCTCCCGTGCTGGAC |
| | | TCCGACGGCTCCTTCTTCCTCTACAGCAAGCTCACCGTGGACAAGA |
| | | GCAGGTGGCAGCAGGGGAACGTCTTCTCATGCTCCGTGATGCATGA |
| | | GGCTCTGCACAACCACTACACGCAGAAGAGCCTCTCCCTGTCTCCG |
| | | GGTAAATGA |
| 41 | CLONE2-H(m1) | QMQLVQSGGGVVRPGGSLRLSCAASGFTFDDYGMSWVRQAPGKGLE |
| | Protein Sequence | WVSGINWNGGSTGYADSVKGRFTISRDNAKNSLYLQMNSLRAEDTA |
| | | LYYCARESSMTTVTTYLLREVGVGLDFDYWGQGTLVTVSSASTKGP |
| | | SVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPP |
| | | SRDELTKNQVSLTCLVKGFYPSDIAVEWESNGQPENNYKTTPPVLD |
| | | SDGSFFLYSKLTVDKSRWQQGNVFSCSVMHEALHNHYTQKSLSLSP |
| | | GK |
| 42 | CLONE2-H(m1) | ATGGAGACAGACACACTCCTGCTATGGGTACTGCTGCTCTGGGTTC |
| | Recombinant | CAGGTTCCACTggcgccggatca |
| | DNA Sequence | CAGATGCAGCTGGTGCAGTCGGGGGGAGGTGTGGTACGGCCTGGGG |
| | (Vector: pYD5- | GGTCCCTGAGACTCTCCTGTGCAGCCTCTGGATTCACCTTTGATGA |
| | hFC(Amp)) | TTATGGCATGAGCTGGGTCCGCCAAGCTCCAGGGAAGGGGCTGGAG |
| | | TGGGTCTCTGGTATTAATTGGAATGGTGGTAGCACAGGTTATGCAG |
| | | ACTCTGTGAAGGGCCGATTCACCATCTCCAGAGACAACGCCAAGAA |
| | | CTCCCTGTATCTGCAAATGAACAGTCTGAGAGCCGAGGACACGGCC |
| | | TTGTATTACTGTGCGAGAGAAAGCTCGATGACTACAGTAACTACGT |
| | | ATCTCCTACGGGAAGTAGGGGTAGGGTTGGACTTTGACTACTGGGG |
| | | CCAGGGCACCCTGGTCACCGTCTCAAGCGCCTCCACCAAGGGCCCA |
| | | TCGGTCTTCCCCCTGGCACCCTCCTCCAAGAGCACCTCTGGGGGCA |
| | | CAGCGGCCCTGGGCTGCCTGGTCAAGGACTACTTCCCCGAACCGGT |
| | | GACGGTGTCGTGGAACTCAGGCGCCCTGACCAGCGGCGTCCACACC |
| | | TTCCCGGCTGTCCTACAGTCCTCAGGACTCTACTCCCTCAGCAGCG |
| | | TAGTGACCGTGCCCTCCAGCAGCTTGGGCACCCAGACCTACATCTG |
| | | CAACGTGAATCACAAGCCCAGCAACACCAAGGTGGACAAGAAAGTT |
| | | GAGCCCAAATCTTGTGACAAAACTCACACATGCCCACCGTGCCCAG |
| | | CACCTGAACTCCTGGGGGGACCGTCAGTCTTCCTCTTCCCCCCAAA |
| | | ACCCAAGGACACCCTCATGATCTCCCGGACCCCTGAGGTCACATGC |
| | | GTGGTGGTGGACGTGAGCCACGAAGACCCTGAGGTCAAGTTCAACT |
| | | GGTACGTGGACGGCGTGGAGGTGCATAATGCCAAGACAAAGCCGCG |
| | | GGAGGAGCAGTACAACAGCACGTACCGTGTGGTCAGCGTCCTCACC |
| | | GTCCTGCACCAGGACTGGCTGAATGGCAAGGAGTACAAGTGCAAGG |
| | | TCTCCAACAAAGCCCTCCCAGCCCCCATCGAGAAAACCATCTCCAA |
| | | AGCCAAAGGGCAGCCCCGAGAACCACAGGTGTACACCCTGCCCCCA |
| | | TCCCGGGATGAGCTGACCAAGAACCAGGTCAGCCTGACCTGCCTGG |
| | | TCAAAGGCTTCTATCCCAGCGACATCGCCGTGGAGTGGGAGAGCAA |
| | | TGGGCAGCCGGAGAACAACTACAAGACCACGCCTCCCGTGCTGGAC |
| | | TCCGACGGCTCCTTCTTCCTCTACAGCAAGCTCACCGTGGACAAGA |
| | | GCAGGTGGCAGCAGGGGAACGTCTTCTCATGCTCCGTGATGCATGA |
| | | GGCTCTGCACAACCACTACACGCAGAAGAGCCTCTCCCTGTCTCCG |
| | | GGTAAATGA |
| 43 | CLONE2-H(m1) | METDTLLLWVLLLWVPGSTGAGS |
| | Recombinant | QMQLVQSGGGVVRPGGSLRLSCAASGFTFDDYGMSWVRQAPGKGLE |
| | Protein Sequence | WVSGINWNGGSTGYADSVKGRFTISRDNAKNSLYLQMNSLRAEDTA |
| | (Vector: pYD5- | LYYCARESSMTTVTTYLLREVGVGLDFDYWGQGTLVTVSSASTKGP |
| | hFC(Amp)) | SVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPP |
| | | SRDELTKNQVSLTCLVKGFYPSDIAVEWESNGQPENNYKTTPPVLD |
| | | SDGSFFLYSKLTVDKSRWQQGNVFSCSVMHEALHNHYTQKSLSLSP |
| | | GK |
| 44 | CLONE2-L(m1) | GAAACGACACTCACGCAGTCTCCAGGCACCCTGTCCTTGTCTCCAG |
| | DNA Sequence | GGGAAAGAGCCACCCTCTCCTGCAGGGCCAGTCAGACTGTTAGCGG |
| | | CAGCTACTTAGCCTGGTACCAGCAGAAACCTGGCCAGCCTCCCAGG |
| | | CTCCTCATCTATGGTGCATCCAGCAGGGCCACTGGCATCCCAGACA |
| | | GGTTCAGTGGCAGTGGGTCCGGGACAGACTTCACTCTCACCATCAG |
| | | CAGACTGGAGCCTGAAGATTTTGCAGTGTATTACTGTCAGGACTAT |
| | | GGTAGCTCACGGACGTTCGGCCAAGGGACCAAGGTGGAACTCAAAC |
| | | GAACTGTGGCTGCACCATCTGTCTTCATCTTCCCGCCATCTGATGA |
| | | GCAGTTGAAATCTGGAACTGCCTCTGTTGTGTGCCTGCTGAATAAC |
| | | TTCTATCCCAGAGAGGCCAAAGTACAGTGGAAGGTGGATAACGCCC |
| | | TCCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAACTCTACGCCTGCGAAGICACCCATCAGG |
| | | GCCTGAGCTCGCCCGTCACAAAGAGCTTCAACAGGGGAGAGTGTTA |
| | | G |
| 45 | CLONE2-L(m1) | ETTLTQSPGTLSLSPGERATLSCRASQTVSGSYLAWYQQKPGQPPR |
| | Protein Sequence | LLIYGASSRATGIPDRFSGSGSGTDFTLTISRLEPEDFAVYYCQDY |
| | | GSSRTFGQGTKVELKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKLYACEVTHQGLSSPVTKSFNRGEC |
| 46 | CLONE2-L(m1) | ATGGAGACAGACACACTCCTGCTATGGGTACTGCTGCTCTGGGTTC |
| | Recombinant | CAGGTTCCACTggcgccggatca |
| | DNA Sequence | GAAACGACACTCACGCAGTCTCCAGGCACCCTGTCCTTGTCTCCAG |
| | (Vector: pYD5- | GGGAAAGAGCCACCCTCTCCTGCAGGGCCAGTCAGACTGTTAGCGG |
| | hFC(Amp)) | CAGCTACTTAGCCTGGTACCAGCAGAAACCTGGCCAGCCTCCCAGG |
| | | CTCCTCATCTATGGTGCATCCAGCAGGGCCACTGGCATCCCAGACA |
| | | GGTTCAGTGGCAGTGGGTCCGGGACAGACTTCACTCTCACCATCAG |
| | | CAGACTGGAGCCTGAAGATTTTGCAGTGTATTACTGTCAGGACTAT |
| | | GGTAGCTCACGGACGTTCGGCCAAGGGACCAAGGTGGAACTCAAAC |
| | | GAACTGTGGCTGCACCATCTGTCTTCATCTTCCCGCCATCTGATGA |
| | | GCAGTTGAAATCTGGAACTGCCTCTGTTGTGTGCCTGCTGAATAAC |
| | | TTCTATCCCAGAGAGGCCAAAGTACAGTGGAAGGTGGATAACGCCC |
| | | TCCAATCGGGTAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAACTCTACGCCTGCGAAGTCACCCATCAGG |
| | | GCCTGAGCTCGCCCGTCACAAAGAGCTTCAACAGGGGAGAGTGTTA |
| | | G |
| 47 | CLONE2-L(m1) | METDTLLLWVLLLWVPGSTGAGS |
| | Recombinant | ETTLTQSPGTLSLSPGERATLSCRASQTVSGSYLAWYQQKPGQPPR |
| | Protein Sequence | LLIYGASSRATGIPDRFSGSGSGTDFTLTISRLEPEDFAVYYCQDY |
| | (Vector: pYD5- | GSSRTFGQGTKVELKRTVAAPSVFIFPPSDEQLKSGTASVVCLLNN |
| | hFC(Amp)) | FYPREAKVQWKVDNALQSGNSQESVTEQDSKDSTYSLSSTLTLSKA |
| | | DYEKHKLYACEVTHQGLSSPVTKSFNRGEC |
| 48 | CLONE3-H(m1) | CAGGTGCAGCTACAGCAGTGGGGCGCAGGACTGTTGAAGCCTTCGG |
| | DNA Sequence | AGACCCTGTCCCTCACCTGCGCTGTCTATGGTGGGTCCTTCAGTGG |
| | | TTACTACTGGAGCTGGATCCGCCAGCCCCCAGGGAAGGGGCTGGAG |
| | | TGGATTGGGGAAATCAATCATAGTGGAAGCACCAACTACAACCCGT |
| | | CCCTCAAGAGTCGAGTCACCATATCAGTAGACACGTCCAAGAACCA |
| | | GTTCTCCCTGAAGCTGAGCTCTGTGACCGCCGCGGACACGGCTGTG |
| | | TATTACTGTGCGAGACGATATTGTAGTGGTGGTAGCTGCTACTTAG |
| | | TTGGAACGGGGTCTGAATTGGACTACTGGGGCCAGGGAACCCTGGT |
| | | CACCGTCTCAAGCGCCTCCACCAAGGGCCCATCGGTCTTCCCCCTG |
| | | GCACCCTCCTCCAAGAGCACCTCTGGGGGCACAGCGGCCCTGGGCT |
| | | GCCTGGTCAAGGACTACTTCCCCGAACCGGTGACGGTGTCGTGGAA |
| | | CTCAGGCGCCCTGACCAGCGGCGTCCACACCTTCCCGGCTGTCCTA |
| | | CAGTCCTCAGGACTCTACTCCCTCAGCAGCGTAGTGACCGTGCCCT |
| | | CCAGCAGCTTGGGCACCCAGACCTACATCTGCAACGTGAATCACAA |
| | | GCCCAGCAACACCAAGGTGGACAAGAAAGTTGAGCCCAAATCTTGT |
| | | GACAAAACTCACACATGCCCACCGTGCCCAGCACCTGAACTCCTGG |
| | | GGGGACCGTCAGTCTTCCTCTTCCCCCCAAAACCCAAGGACACCCT |
| | | CATGATCTCCCGGACCCCTGAGGTCACATGCGTGGTGGTGGACGTG |
| | | AGCCACGAAGACCCTGAGGTCAAGTTCAACTGGTACGTGGACGGCG |
| | | TGGAGGTGCATAATGCCAAGACAAAGCCGCGGGAGGAGCAGTACAA |
| | | CAGCACGTACCGTGTGGTCAGCGTCCTCACCGTCCTGCACCAGGAC |
| | | TGGCTGAATGGCAAGGAGTACAAGTGCAAGGTCTCCAACAAAGCCC |
| | | TCCCAGCCCCCATCGAGAAAACCATCTCCAAAGCCAAAGGGCAGCC |
| | | CCGAGAACCACAGGTGTACACCCTGCCCCCATCCCGGGATGAGCTG |
| | | ACCAAGAACCAGGTCAGCCTGACCTGCCTGGTCAAAGGCTTCTATC |
| | | CCAGCGACATCGCCGTGGAGTGGGAGAGCAATGGGCAGCCGGAGAA |
| | | CACTACAAGACCACGCCTCCCGTGCTGGACTCCGACGGCTCCTTC |
| | | TTCCTCTACAGCAAGCTCACCGTGGACAAGAGCAGGTGGCAGCAGG |
| | | GGAACGTCTTCTCATGCTCCGTGATGCATGAGGCTCTGCACAACCA |
| | | CTACACGCAGAAGAGCCTCTCCCTGTCTCCGGGTAAATGA |
| 49 | CLONE3-H(m1) | QVQLQQWGAGLLKPSETLSLTCAVYGGSFSGYYWSWIRQPPGKGLE |
| | Protein Sequence | WIGEINHSGSTNYNPSLKSRVTISVDTSKNQFSLKLSSVTAADTAV |
| | | YYCARRYCSGGSCYLVGTGSELDYWGQGTLVTVSSASTKGPSVFPL |
| | | APSSKSISGGTAALGCLVKDYFPEPVTVSWNSGALISGVHTFPAVL |
| | | QSSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 50 | CLONE3-H(m1) | ATGGAGACAGACACACTCCTGCTATGGGTACTGCTGCTCTGGGTTC |
| | Recombinant | CAGGTTCCACTggcgccggatca |
| | DNA Sequence | CAGGTGCAGCTACAGCAGTGGGGCGCAGGACTGTTGAAGCCTTCGG |
| | (Vector: pYD5- | AGACCCTGTCCCTCACCTGCGCTGTCTATGGTGGGTCCTTCAGTGG |
| | hFC(Amp)) | TTACTACTGGAGCTGGATCCGCCAGCCCCCAGGGAAGGGGCTGGAG |
| | | TGGATTGGGGAAATCAATCATAGTGGAAGCACCAACTACAACCCGT |
| | | CCCTCAAGAGTCGAGTCACCATATCAGTAGACACGTCCAAGAACCA |
| | | GTTCTCCCTGAAGCTGAGCTCTGTGACCGCCGCGGACACGGCTGTG |
| | | TATTACTGTGCGAGACGATATTGTAGTGGTGGTAGCTGCTACTTAG |
| | | TTGGAACGGGGTCTGAATTGGACTACTGGGGCCAGGGAACCCTGGT |
| | | CACCGTCTCAAGCGCCTCCACCAAGGGCCCATCGGTCTTCCCCCTG |
| | | GCACCCTCCTCCAAGAGCACCTCTGGGGGCACAGCGGCCCTGGGCT |
| | | GCCTGGTCAAGGACTACTTCCCCGAACCGGTGACGGTGTCGTGGAA |
| | | CTCAGGCGCCCTGACCAGCGGCGTCCACACCTTCCCGGCTGTCCTA |
| | | CAGTCCTCAGGACTCTACTCCCTCAGCAGCGTAGTGACCGTGCCCT |
| | | CCAGCAGCTTGGGCACCCAGACCTACATCTGCAACGTGAATCACAA |
| | | GCCCAGCAACACCAAGGTGGACAAGAAAGTTGAGCCCAAATCTTGT |
| | | GACAAAACTCACACATGCCCACCGTGCCCAGCACCTGAACTCCTGG |
| | | GGGGACCGTCAGTCTTCCTCTTCCCCCCAAAACCCAAGGACACCCT |
| | | CATGATCTCCCGGACCCCTGAGGTCACATGCGTGGTGGTGGACGTG |
| | | AGCCACGAAGACCCTGAGGTCAAGTTCAACTGGTACGTGGACGGCG |
| | | TGGAGGTGCATAATGCCAAGACAAAGCCGCGGGAGGAGCAGTACAA |
| | | CAGCACGTACCGTGTGGTCAGCGTCCTCACCGTCCTGCACCAGGAC |
| | | TGGCTGAATGGCAAGGAGTACAAGTGCAAGGTCTCCAACAAAGCCC |
| | | TCCCAGCCCCCATCGAGAAAACCATCTCCAAAGCCAAAGGGCAGCC |
| | | CCGAGAACCACAGGTGTACACCCTGCCCCCATCCCGGGATGAGCTG |
| | | ACCAAGAACCAGGTCAGCCTGACCTGCCTGGTCAAAGGCTTCTATC |
| | | CCAGCGACATCGCCGTGGAGTGGGAGAGCAATGGGCAGCCGGAGAA |
| | | CAACTACAAGACCACGCCTCCCGTGCTGGACTCCGACGGCTCCTTC |
| | | TTCCTCTACAGCAAGCTCACCGTGGACAAGAGCAGGTGGCAGCAGG |
| | | GGAACGTCTTCTCATGCTCCGTGATGCATGAGGCTCTGCACAACCA |
| | | CTACACGCAGAAGAGCCTCTCCCTGTCTCCGGGTAAATGA |
| 51 | CLONE3-H(m1) | METDTLLLWVLLLWVPGSTGAGS |
| | Recombinant | QVQLQQWGAGLLKPSETLSLTCAVYGGSFSGYYWSWIRQPPGKGLE |
| | Protein Sequence | WIGEINHSGSTNYNPSLKSRVTISVDTSKNQFSLKLSSVTAADTAV |
| | (Vector: pYD5- | YYCARRYCSGGSCYLVGTGSELDYWGQGTLVTVSSASTKGPSVFPL |
| | hFC(Amp)) | APSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 52 | CLONE3-L(m1) | GACATCCAGATGACCCAGTCTCCTTCCACCCTGTCTGCATCTGTAG |
| | DNA Sequence | GAGACAGAGTCACCATAACTTGCCGGGCCAGTCAGAGCATTAGTAG |
| | | CTGGTTGGCCTGGTATCAGCAGAAACCAGGGAAAGCCCCTAAACTC |
| | | CTAATCTATAAGGCGTCTAGTTTAGAAATTGGGGTCCCATCAAGGT |
| | | TCAGCGGCAGTGGATATGGGACAGAATTCACTCTCACCATCAGCAG |
| | | CCTGCAGCCTGATGATTTCGCAACTTATTATTGCCTACAGTATAGT |
| | | ACTCATTCGACGTTCGGCCAAGGGACCAGGGTGGAAATCAAACGAA |
| | | CTGTGGCTGCACCATCTGTCTTCATCTTCCCGCCATCTGATGAGCA |
| | | GTTGAAATCTGGAACTGCCTCTGTTGTGTGCCTGCTGAATAACTTC |
| | | TATCCCAGAGAGGCCAAAGTACAGTGGAAGGTGGATAACGCCCTCC |
| | | AATCGGGTAACTCCCAGGAGAGTGTCACAGAGCAGGACAGCAAGGA |
| | | CAGCACCTACAGCCTCAGCAGCACCCTGACGCTGAGCAAAGCAGAC |
| | | TACGAGAAACACAAACTCTACGCCTGCGAAGTCACCCATCAGGGCC |
| | | TGAGCTCGCCCGTCACAAAGAGCTTCAACAGGGGAGAGTGTTAG |
| 53 | CLONE3-L(m1) | DIQMTQSPSTLSASVGDRVTITCRASQSISSWLAWYQQKPGKAPKL |
| | Protein Sequence | LIYKASSLEIGVPSRFSGSGYGTEFTLTISSLQPDDFATYYCLQYS |
| | | THSTFGQGTRVEIKRTVAAPSVFIFPPSDEQLKSGTASVVCLLNNF |
| | | YPREAKVQWKVDNALQSGNSQESVTEQDSKDSTYSLSSTLTLSKAD |
| | | YEKHKLYACEVTHQGLSSPVTKSFNRGEC |
| 54 | CLONE3-L(m1) | ATGGAGACAGACACACTCCTGCTATGGGTACTGCTGCTCTGGGTTC |
| | Recombinant | CAGGTTCCACTggcgccggatca |
| | DNA Sequence | GACATCCAGATGACCCAGTCTCCTTCCACCCTGTCTGCATCTGTAG |
| | (Vector: pYD5- | GAGACAGAGTCACCATAACTTGCCGGGCCAGTCAGAGCATTAGTAG |
| | hFC(Amp)) | CTGGTTGGCCTGGTATCAGCAGAAACCAGGGAAAGCCCCTAAACTC |
| | | CTAATCTATAAGGCGTCTAGTTTAGAAATTGGGGTCCCATCAAGGT |
| | | TCAGCGGCAGTGGATATGGGACAGAATTCACTCTCACCATCAGCAG |
| | | CCTGCAGCCTGATGATTTCGCAACTTATTATTGCCTACAGTATAGT |
| | | ACTCATTCGACGTTCGGCCAAGGGACCAGGGTGGAAATCAAACGAA |
| | | CTGTGGCTGCACCATCTGTCTTCATCTTCCCGCCATCTGATGAGCA |
| | | GTTGAAATCTGGAACTGCCTCTGTTGTGTGCCTGCTGAATAACTTC |
| | | TATCCCAGAGAGGCCAAAGTACAGTGGAAGGTGGATAACGCCCTCC |
| | | AATCGGGTAACTCCCAGGAGAGTGICACAGAGCAGGACAGCAAGGA |
| | | CAGCACCTACAGCCTCAGCAGCACCCTGACGCTGAGCAAAGCAGAC |
| | | TACGAGAAACACAAACTCTACGCCTGCGAAGTCACCCATCAGGGCC |
| | | TGAGCTCGCCCGTCACAAAGAGCTTCAACAGGGGAGAGTGTTAG |
| 55 | CLONE3-L(m1) | METDTLLLWVLLLWVPGSTGAGS |
| | Recombinant | DIQMTQSPSTLSASVGDRVTITCRASQSISSWLAWYQQKPGKAPKL |
| | Protein Sequence | LIYKASSLEIGVPSRFSGSGYGTEFTLTISSLQPDDFATYYCLQYS |
| | (Vector: pYD5- | THSTFGQGTRVEIKRTVAAPSVFIFPPSDEQLKSGTASVVCLLNNF |
| | hFC(Amp)) | YPREAKVQWKVDNALQSGNSQESVTEQDSKDSTYSLSSTLTLSKAD |
| | | YEKHKLYACEVTHQGLSSPVTKSFNRGEC |

The control mouse mAb (clone TIB92) was from ATCC (Manassas, VA, USA). The recombinant human control Ab (cat. No 403502) was from BioLegend (San Diego, CA). In most of the experiments, anti-serpinB13 mAb (clone B29) or control mouse mAb was injected i.p. into pregnant female mice at 50 μg for four consecutive days, starting at gestational day E10.5 (total dose of 200 μg). In the lineage tracing experiments in adult mice, B29 was injected i.p. for 7 days at 100 μg/injection during the first week after STZ treatment (total dose of 700 μg).

Recombinant proteins and their expression. The purified recombinant serpinB13 expressed in baculovirus was obtained from GenScript (Nanjing, China) and used as a competitive inhibitor in the Luminex assay, as well as directly in in vitro cultures of embryonic pancreas explants. To express individual molecules as antigens in the Luminex assay, cDNAs samples encoding human serpinB1 through serpinB13 (serpinB2 and serpinB4 failed to express and were no included in the analysis), green fluorescent protein (Gfp) and secretagogin (Scgn) were subcloned into a pcDNA3.1 Directional V5-His-TOPO vector (cat. no. K490001; Invitrogen, Carlsbad, CA, USA and expressed for 48 hours in 293 cells using lipofectamine 2000 transfection reagent (cat. no. 11668-019; Invitrogen).

Other reagents. Fibronectin (cat. no. F1141-2 mg; Sigma-Aldrich, St. Louis, MO, USA) was used at 50 μg/mL to precoat tissue culture plates to grow ex vivo embryonic pancreas explants. Chicken ovalbumin (cat. no. LS003056; Worthlington, Lakewood, NJ, USA) was used as a control in culture studies with recombinant serpinB13. Cathepsin L was used to stimulate the generation of Ngn3⁺ cells in vitro (cat. no. 1515-CY-010; Biotechne, Minneapolis, MN). The Quant-iT PicoGreen Assay Kit (cat. no. P11496, Thermo Fisher Scientific, Waltham, MA, USA) was used to adjust the amount of released serpinB13 measured by ELISA. DNase I (cat. no. 10104159001; Roche, Basel, Switzerland) and collagenase P (cat. no. 11249002001; Roche) were used to isolate pancreatic islets. The Foxp3/Transcription Factor Staining Buffer Set (cat. no. 00-5523-00; Thermo Fisher Scientific), eBioscience™ Flow Cytometry Staining Buffer (cat no. 00-4222-26; Invitrogen), and IC Fixation Buffer (00-8222-49; Invitrogen) were used to perform staining for FACS analysis. 7-Amino-Actinomycin D (7-AAD) was used to exclude nonviable cells in FACS analysis (cat. no. 51-68981E; BD Biosciences, San Jose, CA). DAPI (4′,6-Diamidino-2-Phenylindole, Dihydrochloride, cat. no. D1306, Thermofisher, 1 g/mL in mounting media) was used to stain nuclei for immunofluorescence microscopy. Tamoxifen (cat. no. T5648-5G; Sigma-Aldrich) was used to induce Cre recombinase expression in Ngn3Cre mice. Streptozotocin (cat. no. S0130-500 mg; Sigma-Aldrich) was used to induce diabetes in C57BL/6J male mice. RIPA buffer (cat. no. 89900; Thermo Fisher Scientific) was used to obtain cell lysates for Western blotting. The BCA protein assay kit (cat. no. 23225; Thermo Fisher Scientific) was used to measure protein concentration in cell lysates. The Mouse Creatinine Assay Kit (cat. no. 80350; CrystalChem, Elk Grove Village, IL, USA) was used to measure creatinine levels in the sera of diabetic mice. E64 protease inhibitor was from Millipore (cat. no. 324890; Billerica, MA, USA). Streptavidin R-PE (cat. no. SA10044, Invitrogen, 1:200) was used to develop the Luminex assay.

Isolation and culture of embryonic pancreas explants. The isolation and culture of embryonic pancreas explants was performed as described elsewhere, with minor modifications.

Preparation of cell suspensions from embryonic pancreas explants. The embryonic pancreases were dissected from the embryos and subjected to treatment with 0.25% Trypsin-EDTA (cat. no. 25200-056; Life Technologies, Carlsbad, CA, USA) followed by gentle pipetting to obtain a single cell suspension. The cells were then fixed and permeabilized, using the Foxp3/Transcription Factor Staining Buffer Set, to permit intracellular staining. Alternatively, to preserve extracellular cell-surface molecules, the embryonic pancreas explants were treated with TrypLE (cat. no. 12605-10; Life Technologies) followed by gentle pipetting. The embryonic pancreatic cells were then fixed for 10 minutes in a mix of equal proportions of 2×IC Fixation Buffer and eBioscience Flow Cytometry Staining Buffer, and finally stained with antibodies for extracellular markers.

Preparation of the islets and single islet-cell suspensions. The adult pancreases were subjected to digestion with collagenase P and passed through the 100-μm strainers to separate the islets from debris. The blindly digested pancreatic samples were manually counted for the number of pancreatic islets under a dissecting microscope with a warm halogen light from below. The islets were defined as any visible, distinct cluster of cells with smooth edges and light- to dark-brown glowing color and diameter greater than 100 μm. The hand-picked islets were then dispersed with CellStripper (cat. no. 25-056-CI; Corning, Corning, NY). The cells were fixed and permeabilized, using the Foxp3/Transcription Factor Staining Buffer Set to permit intracellular staining.

Staining for flow cytometry. BD LSRFortessa X-20, LSR-II and FACSCanto-II were used for FACS analysis. Positive populations of cells were gated and counted using FlowJo ver. 10 software. For the islet cells isolated from adult mice, intracellular staining to detect glucagon and insulin was used to count single-positive as well as double-negative cells. In the cells isolated from embryonic pancreas explants, intracellular staining was performed to detect Ngn3, CK19, and active Notch, and extracellular staining was used to detect the extracellular Notch domain, EpCAM, CD31 and CD45.

Processing of pancreatic tissue for immunofluorescence microscopy. The pancreatic tissues were isolated and fixed either overnight (adult pancreases), or for one hour (embryonic pancreas explants), in 2% PFA (pH 7.4), followed by a two-step saturation process: first in a 30% sucrose solution in PBS and then in optimal cutting temperature (OCT) compound. After complete saturation, the tissues were imbedded in OCT using Cryomold and then snap frozen in an ethanol/dry ice bath. The OCT blocks were serially cut through the entire organ to obtain representative sections every 600 μm (6 to 8 sections per adult pancreas), 50 μm (12 to 14 sections per E16.5 embryonic pancreas), 35 μm (10 to 12 sections per E14.5 embryonic pancreas), or 15 μm (10 to 12 sections per E12.5 embryonic pancreas cultured for 1 to 3 days). For the embryonic linage tracing experiments, three largest sections were taken for the analysis. For all other experiments involving immunofluorescence microscopy, all obtained sections were analyzed.

Staining for immunofluorescence microscopy. In adult mice, pancreatic sections were stained with anti-insulin antibody to measure islet mass. In addition, the skin and pancreas sections from adult mice were used to compare staining patterns with mAbs to serpinB13: clone B29 versus clone B34.

The sections from the pancreases of embryos at age E14.5 and E16.5, and newborn pups (OP), were stained with anti-Ngn3 antibody to determine the total number of endocrine progenitors cells for all sections per explant combined (FIG. 9G, FIG. 17A). Alternatively, the sections representing the pancreases of embryos at age E12.5 (which are considerably smaller) were stained with both anti-Ngn3 and anti-CK19 antibodies to determine the fraction of CK19-positive area that was occupied by Ngn3-positive staining (FIGS. 9D, 10A, 10C, 12D, 12G, 12J and FIGS. 15A, 20B). The following formula was used to adjust for any differences in the pancreas size: (Ngn3⁺ cell number/μm² of CK19⁺ cells)×10⁵.

The sections from the pancreases of embryos at age E11.5 and E16.5 were stained with anti-serpinB13 and anti-CK19 antibodies to determine the level of epithelial expression of serpinB13 during development. In lineage tracing studies determining the number of double positive cells, the pancreatic sections from adult mice and embryos were stained with anti-insulin antibody and anti-GFp antibody, which was used to enhance YFP signal. After staining, images were generated using the Olympus VS120-Fluorescence Virtual Slide Microscope Scanner (Olympus, Tokyo, Japan) and Leica DM5500 B fluorescent microscope.

Image analysis. Embryonic images were processed with a plugin-Trainable Weka Segmentation v3.2.28 (Hamilton, New Zealand) for ImageJ v.1.52jv software. To classify and quantify the images in unbiased fashion, we defined the following four classes for the analysis: Ngn3⁺ cells, the clusters of cytokeratin 19⁺ cells, the negatively stained areas, and background (e.g., the area outside of tissue sample). Alternatively, the Visiopharm version 6.0 software (Visiopharm, Hoerholm, Denmark) with Author module was used to create applications to outline Ngn3⁺ cells or double positive cells expressing YFP and insulin using preprocessing and postprocessing steps, when necessary. In addition, the Engine module of the Visiophram module was used to execute created applications and unbiasedly analyze the images. For the unbiased quantitative analysis of β-cells and islets in the whole pancreatic sections, Visiopharm Author module was trained to recognize insulin-positive β-cell clusters with diameter greater than 50 μm as well as negatively stained section area. In all studies involving microscopy the treatment assignments were blinded to investigators who performed data analysis.

Calculation of the islet mass. In embryos, the β-cell mass was expressed as the percentage of the area of all pancreatic sections combined, that positively stained with anti-insulin antibody. To calculate β-cell mass in adult mice, the percentage insulin-positive area was multiplied by the pancreas weight expressed in milligrams.

Estimating the number of islets and islet cells. The islets were manually counted under a dissecting microscope. To estimate the number of α and β cells, the islet cells were dispersed, intracellularly stained with antibodies to insulin and glucagon, respectively, and their counts measured by FACS.

Diabetes induction and monitoring. C57BL/6 male mice, which are susceptible to STZ-induced diabetes, were subjected to a 6-hour period of bedding removal and fasting with unlimited access to drinking water. At the end of fasting, STZ was dissolved in a freshly prepared buffer (50 mM Sodium Citrate, pH 4.5) and immediately injected i.p. at 150 mg/kg. The glucose levels in tail blood were measured at random every 7 days for 4 weeks. Alternatively, to perform the glucose tolerance test, STZ-treated C57BL/6J male mice were first fasted for 6 hours with unlimited access to drinking water, and then injected i.p. with a 10% D)-(·)-glucose solution (10 μL/g body weight). A glucometer (One-Touch Ultra) was used to monitor glucose levels using tail blood collected before glucose injection, and after injection at 30-min intervals for 2 hours. For glucometer glucose readings “above 600 mg/dL”, the data were extrapolated to the value of 700 mg/dL for algebraic statistical purposes only.

Linage tracing. In the developmental studies, the Rosa26^{EYFP} females were initially set up for overnight breeding with NgnCre^{ERT} males and the following morning examined for the presence of a vaginal plug to indicate embryonic day E0.5. On embryonic day E10.5, E11.5, E12.5 and 13.5, anti-serpinB13 mAb (or control Ab) was injected i.p. at a dose of 50 μg per animal per day (total dose of 200 μg). Two days after the last antibody treatment (E15.5), tamoxifen (20 mg/mL) was injected in a single dose of 3 mg per animal to label the cells. Finally, at 24 hours after tamoxifen injection, the animals were sacrificed, and embryonic pancreas explants were fixed, frozen in OCT blocks, and subjected to IF staining for examination of double-positive (YFP insulin) cells. In the diabetes studies, 8-week old Rosa26^{EYFP}NgnCre^{ERT} males were injected with STZ and treated as described in the legend to FIG. 18A.

Western blotting. Pancreatic tissues were processed from E16.5 embryos as single samples and directly used for lysis. Embryonic pancreas explants from E12.5 embryos were cultured in vitro with anti-serpinB13 mAb (or control Ab) for 24 or 48 hours, then, three explant cultures were combined and lysed. The samples were washed two times in ice-cold PBS and lysed with gentle pipetting for 10 minutes with additional tap-vortexing for 15 minutes in RIPA buffer containing Halt protease inhibitors (cat. no. 1862209, Thermo Fisher Scientific). Equal amounts of protein in each sample were run under reducing conditions on Bis-Tris BOLT gradient gel (4-12%) or NUPAGE Tris-acetate gradient gels (4-12%) (both from Thermo Fisher Scientific), and transferred onto 45 μm nitrocellulose or activated PVDF membranes. The membranes were blocked with 5% skim milk and stained with primary and secondary antibodies, as indicated. The Western blots were developed with SuperSignal West Pico Chemiluminescent Substrate or West Femto Maximum Sensitivity Substrate (cat. nos. 34096 and 34096, respectively; both from Thermo Scientific), and scanned using the ChemiDoc™ MP Imaging System (Bio-Rad, Hercules, VA, USA).

Examination of extracellular serpinB13 by ELISA. The pools of three embryonic pancreas explants or single embryonic heart explants isolated from the wild-type Balb/cJ embryos at E12.5, were cultured in a volume of 110 μL of BME media in a 96-well plate precoated with fibronectin, for 48 hours. After incubation, the culture media was collected and serpinB13 concentration measured using a mouse ELISA assay (cat no. MBS912659; MyBioSource, San Diego, CA, USA). The tissues were harvested to normalize the ELISA results to dsDNA content using the PicoGreen dsDNA Assay Kit (cat. no. P11496; Thermo Fisher Scientific).

Quantitative real-time PCR. Total RNA from embryonic pancreases was extracted using the RNeasy UCP Micro kit (cat. no. 73934; Qiagen, Hilden, Germany) to measure expression of the Notch1 gene. One hundred to 200 μg of the total RNA per group was reverse transcribed to cDNA using the iScript cDNA Synthesis Kit (cat. no. 1708891; Bio-Rad). Quantitative PCR assays were performed on an Applied Biosystems QuantStudio 3 real-time PCR system using cDNA and the Kapa Sybr Fast reagent (cat. no. 0795959100; Roche). Actin-β was used as a reference gene. The primer sequence for Notch1 was as follows: forward—5′CTACAGGGGACACCACCCAC3′ (SEQ ID NO: 87) and reverse—5′ TACAGTACTGACCCGTCCACTC3′ (SEQ ID NO: 88).

The primer sequence for Actin-β was as follows:

forward -
(SEQ ID NO: 89)
5′CTCTGGCTCCTAGCACCATGAAGA3′
and
reverse -
(SEQ ID NO: 90)
5′GTAAAACGCAGCTCAGTAACAGTCCG3′

Examination of cathepsin L protease activity. Cathepsin L Inhibitor Screening Kit from BioVision (cat. no. K161-100; Milpitas, CA, USA) was used according to the manufacturer's recommendations with modifications. To measure impact of binding of antibodies on the inhibitor activity of serpinB13, the two reagents were mixed (e.g., 1 μL of antibody at 1 mg/mL was added to 1 μL of serpinB13 at 100 μg/mL), incubated for one hour at room temperature, and then added to PBS containing BSA at 1 mg/mL and CatL for 15 minutes. The substrate was added as the final step to perform the assay, which was run for 30 min. at 37° C. in kinetic mode using SynergyMx fluorescence microplate reader (BioTek Instruments, Winooski, VT, USA).

Human subjects. SerpinB13 AA were measured in 278 first-degree relatives of TID probands, who were staged for risk for TID (high, intermediate, modest, and low risk) during enrollment in the Diabetes Prevention Trial for Type 1 Diabetes (DPT-1). The criteria defining these risk categories have been described in the DPT-1 protocol. Briefly, the high-risk subjects (n=70, male to female ratio 1.08, mean age 8.6±3.4 years) were defined as having islet cell cytoplasm autoantibodies (ICA), and abnormal first phase insulin response and/or impaired glucose tolerance. The intermediate-risk subjects (n=70, male to female ratio 1.08, mean age 8.4±3.6 years) were defined as having more than one islet autoantibody but no metabolic abnormalities. The modest-risk subjects (n=69, male to female ratio 1.22, mean age 8.7±3.4 years) were defined as being positive for ICA but negative for autoantibodies to native insulin. The low-risk individuals (n=69, male to female ratio 1.09, mean age 8.698±3.4 years) were defined as ICA-negative. Treated subjects from intermediate- and high-risk groups that were enrolled in the DPT-1, were not included in our study. There is no association between serpinB13 AA and protective HLA II haplotype, e.g., HLA-DQB1*0602 or secretion of islet AAs. The Institutional Review Board at the University of Rochester and the University of Minnesota approved all studies with human samples.

Luminex assay. Luminex-based technology was used to measure serpinB13 AA in human samples. Initially, the Luminex beads were precoated with serpinB13, Gfp and Scgn, using precleared lysates of 293 cells that had been transfected with individual cDNAs. Biotinylated mouse anti-human K and 2 chain mAbs (BD Biosciences) (dilution 1:300) and streptavidin (Invitrogen) (dilution 1:200) were used as secondary reagents to measure human serum binding activity to individual antigens. The data were expressed as fluorescence intensity (F.I.) due to serum binding activity in the presence of beads precoated with serpinB13 and after subtracting the average F.I. due to serum binding activity in the presence of beads precoated with control proteins, Gfp and Scgn (e.g. F.I.^{B13}-[F.I.^{GFP}+F.I.^{Segn}]/2). The samples were evaluated based on the level of F.I. and the degree of inhibition of binding to serpin-B13 coated beads with soluble serpinB13 (2.5 μg/mL), compared with the bovine serum albumin (BSA). Specifically, a result was considered positive if binding activity to Luminex bead-bound serpinB13 was 500 to 900 FI units, and the degree of inhibition of this binding with soluble serpinB13 was >25%, or in which binding activity to Luminex bead-bound serpinB13 was ≥900 FI units, regardless of the degree of inhibition of this binding with soluble serpinB13. All samples were run blind on three independent occasions. Subjects with serum samples that produced a positive results three times were considered positive. Subjects with serum samples that produced a negative result on at least one occasion were considered negative.

To determine whether anti-serpin activity is specific, binding of three human recombinant antibodies to serpinB13 was examined for potential cross-reactivity with other clade B serpins. Binding to the beads conjugated with Gfp and Scgn was used to subtract the background and the assay was developed using the same reagents as those described above for measuring serpinB13 AA in serum samples.

Culturing Human Sera with Mouse Embryonic Pancreatic Explants and Immunodepletion Studies.

Serum samples were dialyzed with the Tube-O-DIALYZER Micro, 50 kDa MWCO. (cat. no. 786-614; G-Biosciences, St. Louis, MO, USA) overnight at 4° C., according to the manufacturer's recommendations. In experiments without immunodepletion, the aliquots of 40 μL of dialyzed sera, either positive or negative for serpinB13 AA, were mixed with 70 μL of BME culture media (cat. no. B1522, Sigma) containing 10% FBS, 1% Penicillin-Streptomycin-Glutamine (cat. no. 10378-016, Life Technologies), 50 μg/mL Gentamycin (cat. no. 15750-060, Gibco) and directly added for 48 hours to the in vitro cultured E12.5 pancreatic explants (FIGS. 12D and 12E). Alternatively, the aliquots of 80 μL of dialyzed sera negative for endogenous serpinB13 AA were divided in two parts, reconstituted with 10 g/mL of either recombinant human IgG1 isotype control antibody or recombinant human anti-serpinB13 antibody, and added to the in vitro cultured E12.5 pancreatic explants as described above (FIGS. 12G and 12H). In immunodepletion experiments (FIGS. 12I-12K), the samples were divided in two parts and incubated in 96 well EIA/RIA assay microplates plates (cat. no. CLS3369, Corning), precoated with anti-serpinB13 mAb (clone B29, 10 μg/mL) and either serpinB13 (5 μg/mL), or 2% BSA (sham depletion). After two hours of incubation on a shaker at 4° C., the samples were transferred to the new, same-way treated wells, and incubated for the additional two hours, following which they were premixed with 70 μL of BME culture media and added for 48 hours to the embryonic pancreas explants cultured in vitro, exactly as described above.

Statistics. The data were analyzed using the Prism 8.0 software. Statistical analyses were performed using unpaired two-sided Student's/test, one-way and two-way ANOVA, and the Mantel-Cox test. A P value <0.05 was used to indicate significance. The data are presented as the mean±SEM.

### Antibodies Used in Nonfunctional Studies

Western blotting: Anti-Notch1 (Ala19-Gln526, polyclonal sheep IgG, cat. no. AF5267, Bio-Techne, 1 μg/mL), anti-Notch1 (clone D1E11, rabbit mAb, cat. no. 3608S, Cell Signaling, 1:1000), anti-Ngn3 (clone C-7, mouse mAb, cat. no. sc-374442, Santa Cruz, 0.7 μg/mL), anti-β-tubulin (clone 9F3, rabbit mAb, cat. no. 2128L, Cell Signaling, 1:1000). Secondary antibodies: HRP-conjugated anti-sheep IgG (polyclonal donkey IgG, cat. no. HAF016, Bio-Techne, 1:1000), HRP-conjugated anti-rabbit IgG (polygoclonal goat IgG, cat. no. A27036, Invitrogen, 0.1 μg/mL) and HRP-conjugated anti-mouse IgG (rabbit polygoclonal, cat. no. A27025, Invitrogen, 0.1 μg/mL).

### Flow Cytometry:

Anti-serpinB13 (clone B29, mouse mAb, 2.3 μg/mL), anti-cytokeratin 19 (clone B-1, mouse mAb, cat. no. sc-374192, Santa Cruz, 1 μg/mL), anti-Ngn3 (M-80, rabbit polyclonal, cat. no. Sc25655, Santa Cruz, 1 μg/mL), anti-Notch1-PE (clone 22E5, rat mAb, cat. no. 12-5765-82, eBioscience, 1 μg/mL), anti-activated Notch1 (Val1744, rabbit polyclonal whole antiserum, cat. no. ab8925, Abcam, 1:800), anti-EpCAM-Alexa488 (clone G8.8, rat IgG, cat. no. 118210, BioLegend, 0.625 μg/mL), anti-insulin-Alexa647 (clone T56-706, mouse mAb, cat. no. 565689, BD Biosciences, 1 μg/mL), anti-glucagon-PE (clone U16-850, mouse mAb cat. no. 565860, BD Biosciences, 1:400).

Secondary antibodies: Alexa Fluor 488 goat anti-mouse IgG (H+L) (cat. no. A11001, Invitrogen, 2 μg/mL) and Alexa Fluor 568 goat anti-rabbit IgG (H+L) (cat. no. A11036, Invitrogen, 2 μg/mL). Isotype controls: TIB92 (10-3.6.2, mouse mAb, ATCC, 2.3 μg/mL), rabbit polyclonal IgG (cat. no. 02-6102, Invitrogen), PE rat IgG2a kappa (eBR2a) (cat. no. 12-4321-80, eBiosciense), Alexa Fluor 488 Rat IgG2a, kappa (cat. no. 400525, Biolegend) were diluted to the same concentrations.

IF Microscopy: Anti-serpinB13 (clone B29, mouse mAb, 10 μg/mL), anti-serpinB13 (clone B34, mouse mAb, 10 μg/mL), TIB92 (10-3.6.2, mouse Isotype control, ATCC, 10 μg/mL), anti-cytokeratin 19 (clone B-1, mouse mAb, cat. no. Sc-374192, Santa Cruz, 1 μg/mL), anti-cytokeratin 17/19 (clone D4G2, rabbit mAb, cat. no. 12434S, Cell Signaling, 1:50), anti-cytokeratin 19-Alexa488 (clone EP1580Y, rabbit mAb, cat. no. ab192643, Abcam, 1:100), anti-Ngn3 (M-80, rabbit polyclonal antibody, cat. no. sc-25655, Santa Cruz, 2 μg/mL), anti-insulin (polyclonal guinea pig antibody, cat. no. A0564, DAKO, 2 μg/mL), anti-GFP (rabbit polyclonal antibody, cat. no. A21311, Life Technologies, 2 μg/mL).

Secondary antibodies: Alexa Fluor 488 goat anti-mouse IgG (H+L) (cat. no. A11001, Invitrogen, 2 μg/mL), Alexa Fluor 488 goat anti-rabbit IgG (H+L), (cat no. A11034, Invitrogen, 2 μg/mL), Alexa Fluor 568 goat anti-mouse IgG (H+L) (cat. no. A11031, Invitrogen, 5 μg/mL), Alexa Fluor 594 goat anti-guinea pig IgG (H+L) (cat. no. A11076, Invitrogen, 1 μg/mL), Alexa Fluor 568 goat anti-rabbit IgG (H+L), (cat. no. A11036, Invitrogen, 5 μg/mL), Alexa Fluor 647 goat anti-mouse IgG (H+L) (cat. no. A21235, Invitrogen, 5 μg/mL), and Alexa Fluor 647 goat anti-guinea pig IgG (H+L), (cat. no. A21450, Invitrogen, 5 μg/mL).

Luminex assay: Anti-V5 epitope tag (rabbit polyclonal, cat. no. 903801, BioLegend), biotin anti-human kappa light immunoglobulin chain (clone JDC-12, mouse mAb, cat. no. 555794, BD Biosciences, 1:300), and biotin anti-human lambda light immunoglobulin chain (clone G20-193, mouse mAb, cat. no. 555790, BD Biosciences, 1:300).

### Example 7

Recombinant fully human antibody sequences were developed and are provided below. The CDRs are indicated in bold. The Sequence Identifiers for the CDRs are provided in Table 3 below.

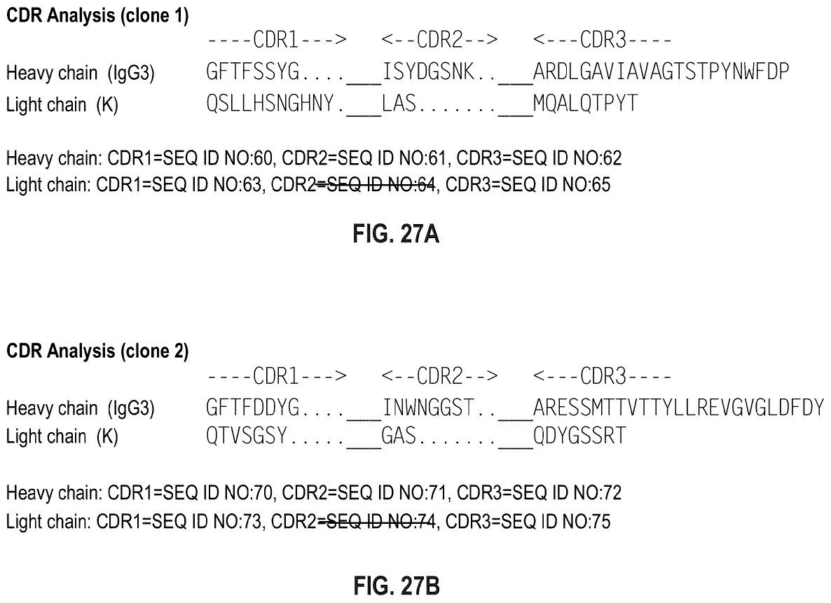

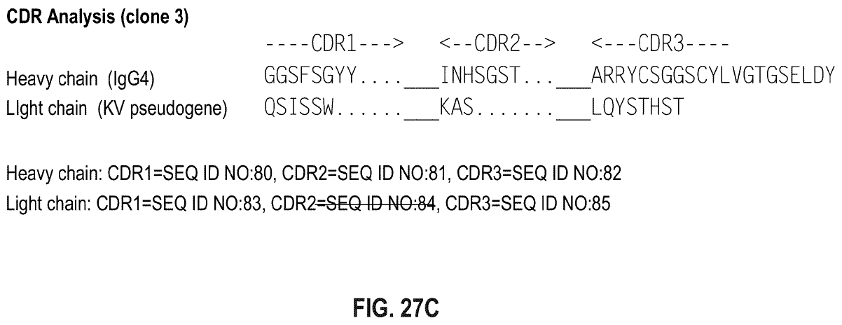

Clone1-H(m1)
DNA: 1380 nt
CAGGTGCAGCTGCAGGAGTCCGGGGGAGGCGTGGTCCAGCCTGGGAGGTCCCTGAGACTCTCCT
GTGCAGCCTCTGGATTCACCTTCAGTAGCTATGGCATGCACTGGGTCCGCCAGGCTCCAGGCAA
GGGGCTGGAGTGGGTGGCAGTTATATCATATGATGGAAGTAATAAATACTATGCAGACTCCGTG
AAGGGCCGATTCACCATCTCCAGAGACAATTCCAAGAACACGCTGTATCTGCAAATGAACAGCC
TGAGAGCCGAGGACACGGCTGTGTATTACTGTGCGAGAGATCTCGGCGCCGTTATAGCAGTGGC
TGGTACTTCCACCCCGTACAACTGGTTCGACCCCTGGGGCCAGGGAACCCTGGTCACCGTCTCA
AGCGCCTCCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGTGGAA
CTCAGGCGCCCTGACCAGCGGCGTCCACACCTTCCCGGCTGTCCTACAGTCCTCAGGACTCTAC
TCCCTCAGCAGCGTAGTGACCGTGCCCTCCAGCAGCTTGGGCACCCAGACCTACATCTGCAACG
TGAATCACAAGCCCAGCAACACCAAGGTGGACAAGAAAGTTGAGCCCAAATCTTGTGACAAAAC
TCACACATGCCCACCGTGCCCAGCACCTGAACTCCTGGGGGGACCGTCAGTCTTCCTCTTCCCC
CCAAAACCCAAGGACACCCTCATGATCTCCCGGACCCCTGAGGTCACATGCGTGGTGGTGGACG
TGAGCCACGAAGACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACAGGTGTACACCCT
GCCCCCATCCCGGGATGAGCTGACCAAGAACCAGGTCAGCCTGACCTGCCTGGTCAAAGGCTTC
TATCCCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTAC
ACGCAGAAGAGCCTCTCCCTGTCTCCGGGTAAATGA (SEQ ID NO: 30)
Protein: 460aa/50 kD
QVQLQESGGGVVQPGRSLRLSCAASGFTFSSYGMHWVRQAPGKGLEWVAVISYDGSNKYYADSV
KGRFTISRDNSKNTLYLQMNSLRAEDTAVYYCARDLGAVIAVAGTSTPYNWFDPWGQGTLVIVS
SASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEALHNHY
TQKSLSLSPGK (SEQ ID NO: 31)
Recombinant Sequence (vector: pYD5-hFC(Amp))
DNA: 1449 nt
ATGGAGACAGACACACTCCTGCTATGGGTACTGCTGCTCTGGGTTCCAGGTTCCACTggcgccg
gatca
CAGGTGCAGCTGCAGGAGTCCGGGGGAGGCGTGGTCCAGCCTGGGAGGTCCCTGAGACTCTCCT
GTGCAGCCTCTGGATTCACCTTCAGTAGCTATGGCATGCACTGGGTCCGCCAGGCTCCAGGCAA
GGGGCTGGAGTGGGTGGCAGTTATATCATATGATGGAAGTAATAAATACTATGCAGACTCCGTG
AAGGGCCGATTCACCATCTCCAGAGACAATTCCAAGAACACGCTGTATCTGCAAATGAACAGCC
TGAGAGCCGAGGACACGGCTGTGTATTACTGTGCGAGAGATCTCGGCGCCGTTATAGCAGTGGC
TGGTACTTCCACCCCGTAGAACTGGTTCGACCCCTGGGGCCAGGGAACCCTGGTCACCGTCTCA
AGCGCCTCCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGTGGAA
CTCAGGCGCCCTGACCAGCGGCGTCCACACCTTCCCGGCTGTCCTACAGTCCTCAGGACTCTAC
TCCCTCAGCAGCGTAGTGACCGTGCCCTCCAGCAGCTTGGGCACCCAGACCTACATCTGCAACG
TGAATCACAAGCCCAGCAACACCAAGGTGGACAAGAAAGTTGAGCCCAAATCTTGTGACAAAAC
TCACACATGCCCACCGTGCCCAGCACCTGAACTCCTGGGGGGACCGTCAGTCTTCCTCTTCCCC
CCAAAACCCAAGGACACCCTCATGATCTCCCGGACCCCTGAGGTCACATGCGTGGTGGTGGACG
TGAGCCACGAAGACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACAGGTGTACACCCT
GCCCCCATCCCGGGATGAGCTGACCAAGAACCAGGTCAGCCTGACCTGCCTGGTCAAAGGCTTC
TATCCCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTAC
ACGCAGAAGAGCCTCTCCCTGTCTCCGGGTAAATGA (vector (italicized) = SEQ
ID NO: 32) (full-length = SEQ ID NO: 33)
Protein: 483aa/52.6 kD
METDTLLLWVLLLWVPGSTGAGS
QVQLQESGGGVVQPGRSLRLSCAASGFTFSSYGMHWVRQAPGKGLEWVAVISYDGSNKYYADSV
KGRFTISRDNSKNTLYLQMNSLRAEDTAVYYCARDLGAVIAVAGTSTPYNWFDPWGQGTLVTVS
SASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEALHNHY
TQKSLSLSPGK (vector (italicized) = SEQ ID NO: 34),
(full-length = SEQ ID NO: 35)
Clone1-L(m1)
DNA: 660 nt
GATGTTGTGATGACTCAGTCTCCACTCTCCCTGCCCGTCACCCCTGGAGAGCCGGCCTCCATCT
CCTGCAGGTCTCGTCAGAGCCTCCTGCATAGCAATGGACACAACTATTTGGGTTGGTACCTGCA
GAAGCCAGGGCAGTCTCCACAGCTCCTGATCTATCTGGCTTCTATTCGGGCCTCCGGGATCCCT
GACAGGTTCAGTGGCAGTGGATCAGGCACAGATTTTACACTGAAAATCAGCAGAGTGGAGGCTG
AGGATGTTGGCGTTTATTACTGCATGCAAGCTCTACAAACACCCTACACTTTTGGCCAGGGGAC
CAAGCTGGAGATCAAGCGAACTGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCT
TCAACAGGGGAGAGTGTTAG (SEQ ID NO: 36)
Protein: 220aa/24 kD
DVVMTQSPLSLPVTPGEPASISCRSRQSLLHSNGHNYLGWYLQKPGQSPQLLIYLASIRASGIP
DRFSGSGSGTDFTLKISRVEAEDVGVYYCMQALQTPYTFGQGTKLEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSFNRGEC (SEQ ID NO: 37)
Recombinant Sequence (Vector: pYD5-hFC(Amp))
DNA: 729 nt
ATGGAGACAGACACACTCCTGCTATGGGTACTGCTGCTCTGGGTTCCAGGTTCCACTggcgccg
gatca
GATGTTGTGATGACTCAGTCTCCACTCTCCCTGCCCGTCACCCCTGGAGAGCCGGCCTCCATCT
CCTGCAGGTCTCGTCAGAGCCTCCTGCATAGCAATGGACACAACTATTTGGGTTGGTACCTGCA
GAAGCCAGGGCAGTCTCCACAGCTCCTGATCTATCTGGCTTCTATTCGGGCCTCCGGGATCCCT
GACAGGTTCAGTGGCAGTGGATCAGGCACAGATTTTACACTGAAAATCAGCAGAGTGGAGGCTG
AGGATGTTGGCGTTTATTACTGCATGCAAGCTCTACAAACACCCTACACTTTTGGCCAGGGGAC
CAAGCTGGAGATCAAGCGAACTGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCT
TCAACAGGGGAGAGTGTTAG (vector (italicized) = SEQ ID NO: 32),
(full-length = SEQ ID NO: 38)
Protein: 243aa/ 26.5 kD
METDTLLLWVLLLWVPGSTGAGS
DVVMTQSPLSLPVTPGEPASISCRSRQSLLHSNGHNYLGWYLQKPGQSPQLLIYLASIRASGIP
DRFSGSGSGTDFTLKISRVEAEDVGVYYCMQALQTPYTFGQGTKLEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSFNRGEC (vector (italicized) = SEQ ID
NO: 34), (full-length = SEQ ID NO: 39)
CLONE2-H(m1)
DNA: 1389 nt
CAGATGCAGCTGGTGCAGTCGGGGGGAGGTGTGGTACGGCCTGGGGGGTCCCTGAGACTCTCCT
GTGCAGCCTCTGGATTCACCTTTGATGATTATGGCATGAGCTGGGTCCGCCAAGCTCCAGGGAA
GGGGCTGGAGTGGGTCTCTGGTATTAATTGGAATGGTGGTAGCACAGGTTATGCAGACTCTGTG
AAGGGCCGATTCACCATCTCCAGAGACAACGCCAAGAACTCCCTGTATCTGCAAATGAACAGTC
TGAGAGCCGAGGACACGGCCTTGTATTACTGTGCGAGAGAAAGCTCGATGACTACAGTAACTAC
GTATCTCCTACGGGAAGTAGGGGTAGGGTTGGACTTTGACTACTGGGGCCAGGGCACCCTGGTC
ACCGTCTCAAGCGCCTCCACCAAGGGCCCATCGGTCTTCCCCCTGGCACCCTCCTCCAAGAGCA
CCTCTGGGGGCACAGCGGCCCTGGGCTGCCTGGTCAAGGACTACTTCCCCGAACCGGTGACGGT
GTCGTGGAACTCAGGCGCCCTGACCAGCGGCGTCCACACCTTCCCGGCTGTCCTACAGTCCTCA
GGACTCTACTCCCTCAGCAGCGTAGTGACCGTGCCCTCCAGCAGCTTGGGCACCCAGACCTACA
TCTGCAACGTGAATCACAAGCCCAGCAACACCAAGGTGGACAAGAAAGTTGAGCCCAAATCTTG
TGACAAAACTCACACATGCCCACCGTGCCCAGCACCTGACTCCTGGGGGGACCGTCAGTCTTC
CTCTTCCCCCCAAAACCCAAGGACACCCTCATGATCTCCCGGACCCCTGAGGTCACATGCGTGG
TGGTGGACGTGAGCCACGAAGACCCTGAGGTCAAGTTCAACTGGTACGTGGACGGCGTGGAGGT
GCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCGAGAACCACAGGT
GTACACCCTGCCCCCATCCCGGGATGAGCTGACCAAGAACCAGGTCAGCCTGACCTGCCTGGTC
AAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGT
GGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTAAATGA (SEQ ID NO: 40)
Protein: 463aa/50.4 kD
QMQLVQSGGGVVRPGGSLRLSCAASGFTFDDYGMSWVRQAPGKGLEWVSGINWNGGSTGYADSV
KGRFTISRDNAKNSLYLQMNSLRAEDTALYYCARESSMTTVTTYLLREVGVGLDFDYWGQGTLV
TVSSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALH
NHYTQKSLSLSPGK (SEQ ID NO: 41)
Recombinant Sequence (Vector: pYD5-hFC(Amp))
DNA: 1458 nt
ATGGAGACAGACACACTCCTGCTATGGGTACTGCTGCTCTGGGTTCCAGGTTCCACTggcgccg
gatca
CAGATGCAGCTGGTGCAGTCGGGGGGAGGTGTGGTACGGCCTGGGGGGTCCCTGAGACTCTCCT
GTGCAGCCTCTGGATTCACCTTTGATGATTATGGCATGAGCTGGGTCCGCCAAGCTCCAGGGAA
GGGGCTGGAGTGGGTCTCTGGTATTAATTGGAATGGTGGTAGCACAGGTTATGCAGACTCTGTG
AAGGGCCGATTCACCATCTCCAGAGACAACGCCAAGAACTCCCTGTATCTGCAAATGAACAGTC
TGAGAGCCGAGGACACGGCCTTGTATTACTGTGCGAGAGAAAGCTCGATGACTAGAGTAACTAC
GTATCTCCTACGGGAAGTAGGGGTAGGGTTGGACTTTGACTACTGGGGCCAGGGCACCCTGGTC
ACCGTCTCAAGCGCCTCCACCAAGGGCCCATCGGTCTTCCCCCTGGCACCCTCCTCCAAGAGCA
CCTCTGGGGGCACAGCGGCCCTGGGCTGCCTGGTCAAGGACTACTTCCCCGAACCGGTGACGGT
GTCGTGGAACTCAGGCGCCCTGACCAGCGGCGTCCACACCTTCCCGGCTGTCCTACAGTCCTCA
GGACTCTACTCCCTCAGCAGCGTAGTGACCGTGCCCTCCAGCAGCTTGGGCACCCAGACCTACA
TCTGCAACGTGAATCACAAGCCCAGCAACACCAAGGTGGACAAGAAAGTTGAGCCCAAATCTTG
TGACAAAACTCACACATGCCCACCGTGCCCAGCACCTGACTCCTGGGGGGACCGTCAGTCTTC
CTCTTCCCCCCAAAACCCAAGGACACCCTCATGATCTCCCGGACCCCTGAGGTCACATGCGTGG
TGGTGGACGTGAGCCACGAAGACCCTGAGGTCAAGTTCAACTGGTACGTGGACGGCGTGGAGGT
GCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCCCGAGAACCACAGGT
GTACACCCTGCCCCCATCCCGGGATGAGCTGACCAAGAACCAGGTCAGCCTGACCTGCCTGGTC
AAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGT
GGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTAAATGA (vector
(italicized) = SEQ ID NO: 32), (full-length = SEQ ID NO: 42)
Protein: 486aa/52.9 kD
METDTLLLWVLLLWVPGSTGAGS
QMQLVQSGGGVVRPGGSLRLSCAASGFTFDDYGMSWVRQAPGKGLEWVSGINWNGGSTGYADSV
KGRFTISRDNAKNSLYLQMNSLRAEDTALYYCARESSMTTVTTYLLREVGVGLDFDYWGQGTLV
TVSSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALH
NHYTQKSLSLSPGK (vector (italicized) = SEQ ID NO: 34), (full-
length = SEQ ID NO: 43)
CLONE2-L(m1)
DNA: 645 nt
GAAACGACACTCACGCAGTCTCCAGGCACCCTGTCCTTGTCTCCAGGGGAAAGAGCCACCCTCT
CCTGCAGGGCCAGTCAGACTGTTAGCGGCAGCTACTTAGCCTGGTACCAGCAGAAACCTGGCCA
GCCTCCCAGGCTCCTCATCTATGGTGCATCCAGCAGGGCCACTGGCATCCCAGACAGGTTCAGT
GGCAGTGGGTCCGGGACAGACTTCACTCTCACCATCAGCAGACTGGAGCCTGAAGATTTTGCAG
TGTATTACTGTCAGGACTATGGTAGCTCACGGACGTTCGGCCAAGGGACCAAGGTGGAACTCAA
ACGAACTGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAACTCTAC
GCCTGCGAAGTCACCCATCAGGGCCTGAGCTCGCCCGTCACAAAGAGCTTCAACAGGGGAGAGT
GTTAG (SEQ ID NO: 44)
Protein: 215aa/23.3 kD
ETTLTQSPGILSLSPGERATLSCRASQTVSGSYLAWYQQKPGQPPRLLIYGASSRATGIPDRFS
GSGSGTDFTLTISRLEPEDFAVYYCQDYGSSRTFGQGTKVELKRTVAAPSVFIFPPSDEQLKSG
TASVVCLLNNFYPREAKVQWKVDNALQSGNSQESVTEQDSKDSTYSLSSTLTLSKADYEKHKLY
ACEVTHQGLSSPVTKSFNRGEC (SEQ ID NO: 45)
Recombinant Sequence (Vector: pYD5-hFC(Amp)
DNA: 714 nt
ATGGAGACAGACACACTCCTGCTATGGGTACTGCTGCTCTGGGTTCCAGGTTCCACTggcgccg
gatca
GAAACGACACTCACGCAGTCTCCAGGCACCCTGTCCTTGTCTCCAGGGGAAAGAGCCACCCTCT
CCTGCAGGGCCAGTCAGACTGTTAGCGGCAGCTACTTAGCCTGGTACCAGCAGAAACCTGGCCA
GCCTCCCAGGCTCCTCATCTATGGTGCATCCAGCAGGGCCACTGGCATCCCAGACAGGTTCAGT
GGCAGTGGGTCCGGGACAGACTTCACTCTCACCATCAGCAGACTGGAGCCTGAAGATTTTGCAG
TGTATTACTGTCAGGACTATGGTAGCTCACGGACGTTCGGCCAAGGGACCAAGGTGGAACTCAA
ACGAACTGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAACTCTAC
GCCTGCGAAGTCACCCATCAGGGCCTGAGCTCGCCCGTCACAAAGAGCTTCAACAGGGGAGAGT
GTTAG (vector (italicized) = SEQ ID NO: 32), (full-length = SEQ
ID NO: 46)
Protein: 238aa/25.8 kD
METDTLLLWVLLLWVPGSTGAGS
ETTLTQSPGILSLSPGERATLSCRASQTVSGSYLAWYQQKPGQPPRLLIYGASSRATGIPDRFS
GSGSGTDFTLTISRLEPEDFAVYYCQDYGSSRTFGQGTKVELKRTVAAPSVFIFPPSDEQLKSG
TASVVCLLNNFYPREAKVQWKVDNALQSGNSQESVTEQDSKDSTYSLSSTLTLSKADYEKHKLY
ACEVTHQGLSSPVTKSFNRGEC (vector (italicized) = SEQ ID NO: 34),
(full-length = SEQ ID NO: 47)
CLONE3-H(m1)
DNA: 1374 nt
CAGGTGCAGCTACAGCAGTGGGGCGCAGGACTGTTGAAGCCTTCGGAGACCCTGTCCCTCACCT
GCGCTGTCTATGGTGGGTCCTTCAGTGGTTACTACTGGAGCTGGATCCGCCAGCCCCCAGGGAA
GGGGCTGGAGTGGATTGGGGAAATCAATCATAGTGGAAGCACCAACTACAACCCGTCCCTCAAG
AGTCGAGTCACCATATCAGTAGACACGTCCAAGAACCAGTTCTCCCTGAAGCTGAGCTCTGTGA
CCGCCGCGGACACGGCTGTGTATTACTGTGCGAGACGATATTGTAGTGGTGGTAGCTGCTACTT
AGTTGGAACGGGGTCTGAATTGGACTACTGGGGCCAGGGAACCCTGGTCACCGTCTCAAGCGCC
TCCACCAAGGGCCCATCGGTCTTCCCCCTGGCACCCTCCTCCAAGAGCACCTCTGGGGGCACAG
CGGCCCTGGGCTGCCTGGTCAAGGACTACTTCCCCGAACCGGTGACGGTGTCGTGGAACTCAGG
CGCCCTGACCAGCGGCGTCCACACCTTCCCGGCTGTCCTACAGTCCTCAGGACTCTACTCCCTC
AGCAGCGTAGTGACCGTGCCCTCCAGCAGCTTGGGCACCCAGACCTACATCTGCAACGTGAATC
ACAAGCCCAGCAACACCAAGGTGGACAAGAAAGTTGAGCCCAAATCTTGTGACAAAACTCACAC
ATGCCCACCGTGCCCAGCACCTGAACTCCTGGGGGGACCGTCAGTCTTCCTCTTCCCCCCAAAA
CCCAAGGACACCCTCATGATCTCCCGGACCCCTGAGGTCACATGCGTGGTGGTGGACGTGAGCC
ACGAAGACCCTGAGGTCAAGTTCAACTGGTACGTGGACGGCGTGGAGGTGCATAATGCCAAGAC
AAAGCCGCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTGTACACCCTGCCCCC
ATCCCGGGATGAGCTGACCAAGAACCAGGTCAGCCTGACCTGCCTGGTCAAAGGCTTCTATCCC
AGCGACATCGCCGTGGAGTGGGAGAGCAATGGGCAGCCGGAGAACAACTACAAGACCACGCCTC
CCGTGCTGGACTCCGACGGCTCCTTCTTCCTCTACAGCAAGCTCACCGTGGACAAGAGCAGGTG
GCAGCAGGGGAACGTCTTCTCATGCTCCGTGATGCATGAGGCTCTGCACAACCACTACACGCAG
AAGAGCCTCTCCCTGTCTCCGGGTAAATGA (SEQ ID NO: 48)
Protein: 458aa/50 kD
QVQLQQWGAGLLKPSETLSLTCAVYGGSFSGYYWSWIRQPPGKGLEWIGEINHSGSTNYNPSLK
SRVTISVDTSKNQFSLKLSSVTAADTAVYYCARRYCSGGSCYLVGTGSELDYWGQGTLVTVSSA
STKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNHYTQ
KSLSLSPGK (SEQ ID NO: 49)
Recombinant Sequence (Vector: pYD5-hFC(Amp))
DNA: 1443 nt
ATGGAGACAGACACACTCCTGCTATGGGTACTGCTGCTCTGGGTTCCAGGTTCCACTggcgccg
gatca
CAGGTGCAGCTACAGCAGTGGGGCGCAGGACTGTTGAAGCCTTCGGAGACCCTGTCCCTCACCT
GCGCTGTCTATGGTGGGTCCTTCAGTGGTTACTACTGGAGCTGGATCCGCCAGCCCCCAGGGAA
GGGGCTGGAGTGGATTGGGGAAATCAATCATAGTGGAAGCACCAACTACAACCCGTCCCTCAAG
AGTCGAGTCACCATATCAGTAGACACGTCCAAGAACCAGTTCTCCCTGAAGCTGAGCTCTGTGA
CCGCCGCGGACACGGCTGTGTATTACTGTGCGAGACGATATTGTAGTGGTGGTAGCTGCTACTT
AGTTGGAACGGGGTCTGAATTGGACTACTGGGGCCAGGGAACCCTGGTCACCGTCTCAAGCGCC
TCCACCAAGGGCCCATCGGTCTTCCCCCTGGCACCCTCCTCCAAGAGCACCTCTGGGGGCACAG
CGGCCCTGGGCTGCCTGGTCAAGGACTACTTCCCCGAACCGGTGACGGTGTCGTGGAACTCAGG
CGCCCTGACCAGCGGCGTCCACACCTTCCCGGCTGTCCTACAGTCCTCAGGACTCTACTCCCTC
AGCAGCGTAGTGACCGTGCCCTCCAGCAGCTTGGGCACCCAGACCTACATCTGCAACGTGAATC
ACAAGCCCAGCAACACCAAGGTGGACAAGAAAGTTGAGCCCAAATCTTGTGACAAAACTCACAC
ATGCCCACCGTGCCCAGCACCTGAACTCCTGGGGGGACCGTCAGTCTTCCTCTTCCCCCCAAAA
CCCAAGGACACCCTCATGATCTCCCGGACCCCTGAGGTCACATGCGTGGTGGTGGACGTGAGCC
ACGAAGACCCTGAGGTCAAGTTCAACTGGTACGTGGACGGCGTGGAGGTGCATAATGCCAAGAC
AAAGCCGCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTGTACACCCTGCCCCC
ATCCCGGGATGAGCTGACCAAGAACCAGGTCAGCCTGACCTGCCTGGTCAAAGGCTTCTATCCC
AGCGACATCGCCGTGGAGTGGGAGAGCAATGGGCAGCCGGAGAACAACTACAAGACCACGCCTC
CCGTGCTGGACTCCGACGGCTCCTTCTTCCTCTACAGCAAGCTCACCGTGGACAAGAGCAGGTG
GCAGCAGGGGAACGTCTTCTCATGCTCCGTGATGCATGAGGCTCTGCACAACCACTACACGCAG
AAGAGCCTCTCCCTGTCTCCGGGTAAATGA (vector (italicized) = SEQ ID
NO: 32), (full-length = SEQ ID NO: 50)
Protein: 481aa/ 52.4 kD
METDTLLLWVLLLWVPGSTGAGS
QVQLQQWGAGLLKPSETLSLTCAVYGGSFSGYYWSWIRQPPGKGLEWIGEINHSGSTNYNPSLK
SRVTISVDTSKNQFSLKLSSVTAADTAVYYCARRYCSGGSCYLVGTGSELDYWGQGTLVTVSSA
STKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNHYTQ
KSLSLSPGK (vector (italicized) = SEQ ID NO: 34), (full-length =
SEQ ID NO: 51)
CLONE3-L(m1)
DNA: 642 nt
GACATCCAGATGACCCAGTCTCCTTCCACCCTGTCTGCATCTGTAGGAGACAGAGTCACCATAA
CTTGCCGGGCCAGTCAGAGCATTAGTAGCTGGTTGGCCTGGTATCAGCAGAAACCAGGGAAAGC
CCCTAAACTCCTAATCTATAAGGCGTCTAGTTTAGAAATTGGGGTCCCATCAAGGTTCAGCGGC
AGTGGATATGGGACAGAATTCACTCTCACCATCAGCAGCCTGCAGCCTGATGATTTCGCAACTT
ATTATTGCCTACAGTATAGTACTCATTCGACGTTCGGCCAAGGGACCAGGGTGGAAATCAAACG
AACTGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAACTCTACGCC
TGCGAAGTCACCCATCAGGGCCTGAGCTCGCCCGTCACAAAGAGCTTCAACAGGGGAGAGTGTT
AG (SEQ ID NO: 52)
Protein: 214aa/23.4 kD
DIQMTQSPSTLSASVGDRVTITCRASQSISSWLAWYQQKPGKAPKLLIYKASSLEIGVPSRFSG
SGYGTEFTLTISSLQPDDFATYYCLQYSTHSTFGQGTRVEIKRTVAAPSVFIFPPSDEQLKSGT
ASVVCLLNNFYPREAKVQWKVDNALQSGNSQESVTEQDSKDSTYSLSSTLTLSKADYEKHKLYA
CEVTHQGLSSPVTKSFNRGEC (SEQ ID NO: 53)
Recombinant Sequence (Vector: pYD5-hFC(Amp))
DNA: 711 nt
ATGGAGACAGACACACTCCTGCTATGGGTACTGCTGCTCTGGGTTCCAGGTTCCACTggcgccg
gatca
GACATCCAGATGACCCAGTCTCCTTCCACCCTGTCTGCATCTGTAGGAGACAGAGTCACCATAA
CTTGCCGGGCCAGTCAGAGCATTAGTAGCTGGTTGGCCTGGTATCAGCAGAAACCAGGGAAAGC
CCCTAAACTCCTAATCTATAAGGCGTCTAGTTTAGAAATTGGGGTCCCATCAAGGTTCAGCGGC
AGTGGATATGGGACAGAATTCACTCTCACCATCAGCAGCCTGCAGCCTGATGATTTCGCAACTT
ATTATTGCCTACAGTATAGTACTCATTCGACGTTCGGCCAAGGGACCAGGGTGGAAATCAAACG
AACTGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAACTCTACGCC
TGCGAAGTCACCCATCAGGGCCTGAGCTCGCCCGTCACAAAGAGCTTCAACAGGGGAGAGTGTT
AG (vector (italicized) = SEQ ID NO: 32), (full-length = SEQ ID
NO: 54)
Protein: 237aa/26 kD
METDTLLLWVLLLWVPGSTGAGS
DIQMTQSPSTLSASVGDRVTITCRASQSISSWLAWYQQKPGKAPKLLIYKASSLEIGVPSRFSG
SGYGTEFTLTISSLQPDDFATYYCLQYSTHSTFGQGTRVEIKRTVAAPSVFIFPPSDEQLKSGT
ASVVCLLNNFYPREAKVQWKVDNALQSGNSQESVTEQDSKDSTYSLSSTLTLSKADYEKHKLYA
CEVTHQGLSSPVTKSFNRGEC (vector (italicized) = SEQ ID NO: 32),
(full-length = SEQ ID NO: 55)
Clone 1
Heavy chain variable region sequence
CAGGTGCAGCTGCAGGAGTCCGGGGGAGGCGTGGTCCAGCCTGGGAGGTCCCTGAGACTCTCCT
GTGCAGCCTCTGGATTCACCTTCAGTAGCTATGGCATGCACTGGGTCCGCCAGGCTCCAGGCAA
GGGGCTGGAGTGGGTGGCAGTTATATCATATGATGGAAGTAATAAATACTATGCAGACTCCGTG
AAGGGCCGATTCACCATCTCCAGAGACAATTCCAAGAACACGCTGTATCTGCAAATGAACAGCC
TGAGAGCCGAGGACACGGCTGTGTATTACTGTGCGAGAGATCTCGGCGCCGTTATAGCAGTGGC
TGGTACTTCCACCCCGTACAACTGGTTCGACCCCTGGGGCCAGGGAACCCTGGTCACCGTCTC
(SEQ ID NO: 56)
Translated protein:
QVQLQESGGGVVQPGRSLRLSCAASGFTFSSYGMHWVRQAPGKGLEWVAVISYDGSNKYYADSV
KGRFTISRDNSKNTLYLQMNSLRAEDTAVYYCARDLGAVIAVAGTSTPYNWFDPWGQGTLVTVS
(SEQ ID NO: 57)
Light chain variable region sequence
GATGTTGTGATGACTCAGTCTCCACTCTCCCTGCCCGTCACCCCTGGAGAGCCGGCCTCCATCT
CCTGCAGGTCTCGTCAGAGCCTCCTGCATAGCAATGGACACAACTATTTGGGTTGGTACCTGCA
GAAGCCAGGGCAGTCTCCACAGCTCCTGATCTATCTGGCTTCTATTCGGGCCTCCGGGATCCCT
GACAGGTTCAGTGGCAGTGGATCAGGCACAGATTTTACACTGAAAATCAGCAGAGTGGAGGCTG
AGGATGTTGGCGTTTATTACTGCATGCAAGCTCTACAAACACCCTACACTTTTGGCCAGGGGAC
CAAGCTGGAGATCAA (SEQ ID NO: 58)
Translated protein:
DVVMTQSPLSLPVTPGEPASISCRSRQSLLHSNGHNYLGWYLQKPGQSPQLLIYLASIRASGIP
DRFSGSGSGTDFTLKISRVEAEDVGVYYCMQALQTPYTFGQGTKLEIK (SEQ ID NO: 59)
The CDR Analysis for clone 1 is provided in FIG. 27A.

Clone 2
Heavy chain variable region sequence
(SEQ ID NO: 66)
CAGATGCAGCTGGTGCAGTCGGGGGGAGGTGTGGTACGGCCTGGGGGGTC
CCTGAGACTCTCCTGTGCAGCCTCTGGATTCACCTTTGATGATTATGGCA
TGAGCTGGGTCCGCCAAGCTCCAGGGAAGGGGCTGGAGTGGGTCTCTGGT
ATTAATTGGAATGGTGGTAGCACAGGTTATGCAGACTCTGTGAAGGGCCG
ATTCACCATCTCCAGAGACAACGCCAAGAACTCCCTGTATCTGCAAATGA
ACAGTCTGAGAGCCGAGGACACGGCCTTGTATTACTGTGCGAGAGAAAGC
TCGATGACTACAGTAACTACGTATCTCCTACGGGAAGTAGGGGTAGGGTT
GGACTTTGACTACTGGGGCCAGGGCACCCTGGTCACCGTCTC
Translated protein:
(SEQ ID NO: 67)
QMQLVQSGGGVVRPGGSLRLSCAASGFTFDDYGMSWVRQAPGKGLEWVSG
INWNGGSTGYADSVKGRFTISRDNAKNSLYLQMNSLRAEDTALYYCARES
SMTTVTTYLLREVGVGLDFDYWGQGTLVTVS
Light chain variable region sequence
(SEQ ID NO: 68)
GAAACGACACTCACGCAGTCTCCAGGCACCCTGTCCTTGTCTCCAGGGGA
AAGAGCCACCCTCTCCTGCAGGGCCAGTCAGACTGTTAGCGGCAGCTACT
TAGCCTGGTACCAGCAGAAACCTGGCCAGCCTCCCAGGCTCCTCATCTAT
GGTGCATCCAGCAGGGCCACTGGCATCCCAGACAGGTTCAGTGGCAGTGG
GTCCGGGACAGACTTCACTCTCACCATCAGCAGACTGGAGCCTGAAGATT
TTGCAGTGTATTACTGTCAGGACTATGGTAGCTCACGGACGTTCGGCCAA
GGGACCAAGGTGGAACTCAAAC
Translated protein:
(SEQ ID NO: 69)
ETTLTQSPGILSLSPGERATLSCRASQTVSGSYLAWYQQKPGQPPRLLIY
GASSRATGIPDRFSGSGSGTDFTLTISRLEPEDFAVYYCQDYGSSRTFGQ
GTKVELK

Clone 3
Heavy chain variable region sequence
(SEQ ID NO: 76)
CAGGTGCAGCTACAGCAGTGGGGCGCAGGACTGTTGAAGCCTTCGGAGAC
CCTGTCCCTCACCTGCGCTGTCTATGGTGGGTCCTTCAGTGGTTACTACT
GGAGCTGGATCCGCCAGCCCCCAGGGAAGGGGCTGGAGTGGATTGGGGAA
ATCAATGATAGTGGAAGCACCAACTACAACCCGTCCCTCAAGAGTCGAGT
CACCATATCAGTAGACACGTCCAAGAACCAGTTCTCCCTGAAGCTGAGCT
CTGTGACCGCCGCGGACACGGCTGTGTATTACTGTGCGAGACGATATTGT
AGTGGTGGTAGCTGCTACTTAGTTGGAACGGGGTCTGAATTGGACTACTG
GGGCCAGGGAACCCTGGTCACCGTCTC
Translated protein:
(SEQ ID NO: 77)
QVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCARRYC
SGGSCYLVGTGSELDYWGQGTLVTVS
Light chain variable region sequence
(SEQ ID NO: 78)
GACATCCAGATGACCCAGTCTCCTTCCACCCTGTCTGCATCTGTAGGAGA
CAGAGTCACCATAACTTGCCGGGCCAGTCAGAGCATTAGTAGCTGGTTGG
CCTGGTATCAGCAGAAACCAGGGAAAGCCCCTAAACTCCTAATCTATAAG
GCGTCTAGTTTAGAAATTGGGGTCCCATCAAGGTTCAGCGGCAGTGGATA
TGGGACAGAATTCACTCTCACCATCAGCAGCCTGCAGCCTGATGATTTCG
CAACTTATTATTGCCTACAGTATAGTACTCATTCGACGTTCGGCCAAGGG
ACCAGGGTGGAAATCAAAC
Translated protein:
(SEQ ID NO: 79)
DIQMIQSPSTLSASVGDRVTITCRASQSISSWLAWYQQKPGKAPKLLIY
KASSLEIGVPSRFSGSGYGTEFTLTISSLQPDDFATYYCLQYSTHSTFG
QGTRVEIK

The CDR Analysis for clone 3 is provided in FIG. 27C.

TABLE 3
Table of Sequences

| SEQ | | |
|---|---|---|
| ID | | |
| NO | Description | Sequence |
| 56 | Clone 1 heavy | CAGGTGCAGCTGCAGGAGTCCGGGGGAGGCGTGGTCCAGCCTGGGA |
| | chain variable | GGTCCCTGAGACTCTCCTGTGCAGCCTCTGGATTCACCTTCAGTAG |
| | sequence (DNA) | CTATGGCATGCACTGGGTCCGCCAGGCTCCAGGCAAGGGGCTGGAG |
| | | TGGGTGGCAGTTATATCATATGATGGAAGTAATAAATACTATGCAG |
| | | ACTCCGTGAAGGGCCGATTCACCATCTCCAGAGACAATTCCAAGAA |
| | | CACGCTGTATCTGCAAATGAACAGCCTGAGAGCCGAGGACACGGCT |
| | | GTGTATTACTGTGCGAGAGATCTCGGCGCCGTTATAGCAGTGGCTG |
| | | GTACTTCCACCCCGTACAACTGGTTCGACCCCTGGGGCCAGGGAAC |
| | | CCTGGTCACCGTCTC |
| 57 | Clone 1 heavy | QVQLQESGGGVVQPGRSLRLSCAASGFTFSSYGMHWVRQAPGKGLE |
| | chain variable | WVAVISYDGSNKYYADSVKGRFTISRDNSKNTLYLQMNSLRAEDTA |
| | sequence | VYYCARDLGAVIAVAGTSTPYNWFDPWGQGTLVTVS |
| | Translated | |
| | protein: | |
| 58 | Clone 1 Light | GATGTTGTGATGACTCAGTCTCCACTCTCCCTGCCCGTCACCCCTG |
| | chain | GAGAGCCGGCCTCCATCTCCTGCAGGTCTCGTCAGAGCCTCCTGCA |
| | variable | TAGCAATGGACACAACTATTTGGGTTGGTACCTGCAGAAGCCAGGG |
| | region | CAGTCTCCACAGCTCCTGATCTATCTGGCTTCTATTCGGGCCTCCG |
| | sequence | GGATCCCTGACAGGTTCAGTGGCAGTGGATCAGGCACAGATTTTAC |
| | | ACTGAAAATCAGCAGAGTGGAGGCTGAGGATGTTGGCGTTTATTAC |
| | | TGCATGCAAGCTCTACAAACACCCTACACTTTTGGCCAGGGGACCA |
| | | AGCTGGAGATCAA |
| 59 | Translated | DVVMTQSPLSLPVTPGEPASISCRSRQSLLHSNGHNYLGWYLQKPG |
| | protein: | QSPQLLIYLASIRASGIPDRFSGSGSGTDFTLKISRVEAEDVGVYY |
| | | CMQALQTPYTFGQGTKLEIK |
| 60 | Clone 1 | GFTFSSYG |
| | Heavy chain | |
| | (IgG3) CDR1 | |
| 61 | Clone 1 | ISYDGSNK |
| | Heavy chain | |
| | (IgG3) CDR2 | |
| 62 | Clone 1 | ARDLGAVIAVAGTSTPYNWFDP |
| | Heavy chain | |
| | (IgG3) CDR3 | |
| 63 | Clone 1 | QSLLHSNGHNY |
| | Light chain | |
| | (K) CDR1 | |
| 64 | Clone 1 | LAS |
| | Light chain | |
| | (K) CDR2 | |
| 65 | Clone 1 | MQALQTPYT |
| | Light chain | |
| | (K) CDR3 | |
| 66 | Clone 2 | CAGATGCAGCTGGTGCAGTCGGGGGGAGGTGTGGTACGGCCTGGGG |
| | Heavy chain | GGTCCCTGAGACTCTCCTGTGCAGCCTCTGGATTCACCTTTGATGA |
| | variable | TTATGGCATGAGCTGGGTCCGCCAAGCTCCAGGGAAGGGGCTGGAG |
| | region | TGGGTCTCTGGTATTAATTGGAATGGTGGTAGCACAGGTTATGCAG |
| | sequence | ACTCTGTGAAGGGCCGATTCACCATCTCCAGAGACAACGCCAAGAA |
| | | CTCCCTGTATCTGCAAATGAACAGTCTGAGAGCCGAGGACACGGCC |
| | | TTGTATTACTGTGCGAGAGAAAGCTCGATGACTACAGTAACTACGT |
| | | ATCTCCTACGGGAAGTAGGGGTAGGGTTGGACTTTGACTACTGGGG |
| | | CCAGGGCACCCTGGTCACCGTCTC |
| 67 | Translated | QMQLVQSGGGVVRPGGSLRLSCAASGFTFDDYGMSWVRQAPGKGLE |
| | protein: | WVSGINWNGGSTGYADSVKGRFTISRDNAKNSLYLQMNSLRAEDTA |
| | | LYYCARESSMTTVTTYLLREVGVGLDFDYWGQGTLVTVS |
| 68 | Light chain | GAAACGACACTCACGCAGTCTCCAGGCACCCTGTCCTTGTCTCCAG |
| | variable | GGGAAAGAGCCACCCTCTCCTGCAGGGCCAGTCAGACTGTTAGCGG |
| | region | CAGCTACTTAGCCTGGTACCAGCAGAAACCTGGCCAGCCTCCCAGG |
| | sequence | CTCCTCATCTATGGTGCATCCAGCAGGGCCACTGGCATCCCAGACA |
| | | GGTTCAGTGGCAGTGGGTCCGGGACAGACTTCACTCTCACCATCAG |
| | | CAGACTGGAGCCTGAAGATTTTGCAGTGTATTACTGTCAGGACTAT |
| | | GGTAGCTCACGGACGTTCGGCCAAGGGACCAAGGTGGAACTCAAAC |
| 69 | Translated | ETTLTQSPGTLSLSPGERATLSCRASQTVSGSYLAWYQQKPGQPPR |
| | protein: | LLIYGASSRATGIPDRFSGSGSGTDFTLTISRLEPEDFAVYYCQDY |
| | | GSSRTFGQGTKVELK |
| 70 | Clone 2 | GFTFDDYG |
| | Heavy chain | |
| | (IgG3) CDR1 | |
| 71 | Clone 2 | INWNGGST |
| | Heavy chain | |
| | (IgG3) CDR2 | |
| 72 | Clone 2 | ARESSMTTVTTYLLREVGVGLDFDY |
| | Heavy chain | |
| | (IgG3) CDR3 | |
| 73 | Clone 2 | QTVSGSY |
| | Light chain | |
| | (K) CDR1 | |
| 74 | Clone 2 | GAS |
| | Light chain | |
| | (K) CDR2 | |
| 75 | Clone 2 | QDYGSSRT |
| | Light chain | |
| | (K) CDR3 | |
| 76 | Clone 3 | CAGGTGCAGCTACAGCAGTGGGGCGCAGGACTGTTGAAGCCTTCGG |
| | Heavy chain | AGACCCTGTCCCTCACCTGCGCTGTCTATGGTGGGTCCTTCAGTGG |
| | variable | TTACTACTGGAGCTGGATCCGCCAGCCCCCAGGGAAGGGGCTGGAG |
| | region | TGGATTGGGGAAATCAATCATAGTGGAAGCACCAACTACAACCCGT |
| | sequence | CCCTCAAGAGTCGAGTCACCATATCAGTAGACACGTCCAAGAACCA |
| | | GTTCTCCCTGAAGCTGAGCTCTGTGACCGCCGCGGACACGGCTGTG |
| | | TATTACTGTGCGAGACGATATTGTAGTGGTGGTAGCTGCTACTTAG |
| | | TTGGAACGGGGTCTGAATTGGACTACTGGGGCCAGGGAACCCTGGT |
| | | CACCGTCTC |
| 77 | Translated | QVQLQQWGAGLLKPSETLSLTCAVYGGSFSGYYWSWIRQPPGKGLE |
| | protein: | WIGEINHSGSTNYNPSLKSRVTISVDTSKNQFSLKLSSVTAADTAV |
| | | YYCARRYCSGGSCYLVGTGSELDYWGQGTLVTVS |
| 78 | Light chain | GACATCCAGATGACCCAGTCTCCTTCCACCCTGTCTGCATCTGTAG |
| | variable | GAGACAGAGTCACCATAACTTGCCGGGCCAGTCAGAGCATTAGTAG |
| | region | CTGGTTGGCCTGGTATCAGCAGAAACCAGGGAAAGCCCCTAAACTC |
| | sequence | CTAATCTATAAGGCGTCTAGTTTAGAAATTGGGGTCCCATCAAGGT |
| | | TCAGCGGCAGTGGATATGGGACAGAATTCACTCTCACCATCAGCAG |
| | | CCTGCAGCCTGATGATTTCGCAACTTATTATTGCCTACAGTATAGT |
| | | ACTCATTCGACGTTCGGCCAAGGGACCAGGGTGGAAATCAAAC |
| 79 | Translated | DIQMTQSPSTLSASVGDRVTITCRASQSISSWLAWYQQKPGKAPKL |
| | protein: | LIYKASSLEIGVPSRFSGSGYGTEFTLTISSLQPDDFATYYCLQYS |
| | | THSTFGQGTRVEIK |
| 80 | Clone 3 | GGSFSGYY |
| | Heavy chain | |
| | (IgG4) CDR1 | |
| 81 | Clone 3 | INHSGST |
| | Heavy chain | |
| | (IgG4) CDR2 | |
| 82 | Clone 3 | ARRYCSGGSCYLVGTGSELDY |
| | Heavy chain | |
| | (IgG4) CDR3 | |
| 83 | Clone 3 | QSISSW |
| | Light chain | |
| | (KV | |
| | pseudogene) | |
| | CDR1 | |
| 84 | Clone 3 | KAS |
| | Light chain | |
| | (KV | |
| | pseudogene) | |
| | CDR2 | |
| 85 | Clone 3 | LQYSTHST |
| | Light chain | |
| | (KV | |
| | pseudogene) | |
| | CDR3 | |

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the term “about” means approximately ±10%.

Embodiments of this invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An isolated antibody or antigen-binding fragment thereof that binds to OVA-serine proteinase inhibitor (serpin) B13 and comprises a heavy chain, wherein the heavy chain comprises a heavy chain CDR1, a heavy chain CDR2, a heavy chain CDR3, and a light chain, wherein the light chain comprises a light chain CDR1, a light chain CDR2, and a light chain CDR3, wherein:
(i) the heavy chain CDR1 comprises the amino acid sequence of SEQ ID NO:1 with a substitution at one, two or three amino acid positions, SEQ ID NO:2 with a substitution at one, two or three amino acid positions, SEQ ID NO:26 with a substitution at one, two or three amino acid positions, SEQ ID NO:60 with a substitution at one, two or three amino acid positions, SEQ ID NO:70 with a substitution at one, two or three amino acid positions or SEQ ID NO:80 with a substitution at one, two or three amino acid positions,
(ii) the heavy chain CDR2 comprises the amino acid sequence of SEQ ID NO:4 with a substitution at one, two or three amino acid positions, SEQ ID NO:27 with a substitution at one, two or three amino acid positions, SEQ ID NO:61 with a substitution at one, two or three amino acid positions, SEQ ID NO:71 with a substitution at one, two or three amino acid positions or SEQ ID NO:81 with a substitution at one, two or three amino acid positions,
(iii) the heavy chain CDR3 comprises the amino acid sequence of SEQ ID NO:6 with a substitution at one, two or three amino acid positions, SEQ ID NO:28 with a substitution at one, two or three amino acid positions, SEQ ID NO:62 with a substitution at one, two or three amino acid positions, SEQ ID NO:72 with a substitution at one, two or three amino acid positions or SEQ ID NO:82 with a substitution at one, two or three amino acid positions,
(iv) the light chain CDR1 comprises the amino acid sequence of SEQ ID NO:8 with a substitution at one, two or three amino acid positions, SEQ ID NO:29 with a substitution at one, two or three amino acid positions, SEQ ID NO:63 with a substitution at one, two or three amino acid positions, SEQ ID NO:73 with a substitution at one, two or three amino acid positions or SEQ ID NO:83 with a substitution at one, two or three amino acid positions,
(v) the light chain CDR2 comprises the amino acid sequence of SEQ ID NO:10 with a substitution at one, two or three amino acid positions, SEQ ID NO:64 with a substitution at one, two or three amino acid positions, SEQ ID NO:74 with a substitution at one, two or three amino acid positions or SEQ ID NO:84 with a substitution at one, two or three amino acid positions, and
(vi) the light chain CDR3 comprises the amino acid sequence of SEQ ID NO:12 with a substitution at one, two or three amino acid positions, SEQ ID NO:65 with a substitution at one, two or three amino acid positions, SEQ ID NO:75 with a substitution at one, two or three amino acid positions or SEQ ID NO:85 with a substitution at one, two or three amino acid positions.

2. The isolated antibody or antigen-binding fragment thereof of claim 1, wherein the isolated antibody or antigen-binding fragment thereof is a humanized antibody.

3. The isolated antibody or antigen-binding fragment of claim 2, wherein the heavy chain comprises the amino acid sequence of SEQ ID NO: 18 with a substitution at one, two or three amino acid positions.

4. The isolated antibody or antigen-binding fragment of claim 2, wherein the light chain comprises the amino acid sequence of SEQ ID NO:20 with a substitution at one, two or three amino acid positions.

5. The isolated antibody or antigen-binding fragment thereof of claim 1, wherein the isolated antibody or antigen-binding fragment thereof is a chimeric antibody.

6. The isolated antibody or antigen-binding fragment of claim 5, wherein the heavy chain comprises the amino acid sequence of SEQ ID NO:22 with a substitution at one, two or three amino acid positions.

7. The isolated antibody or antigen-binding fragment of claim 5, wherein the light chain comprises the amino acid sequence of SEQ ID NO:24 with a substitution at one, two or three amino acid positions.

8. The antigen-binding fragment of claim 1, wherein the antigen-binding fragment is selected from the group consisting of a Fab fragment, a F(ab′)2 fragment, a scFv fragment, and a sc(Fv)2 diabody.

9. A composition comprising at least one isolated antibody or antigen-binding fragment of claim 1.

10. A method of inhibiting an OVA-serine proteinase inhibitor (serpin) B13-related disorder in a subject, the method comprising administering to the subject the isolated antibody or antigen-binding fragment thereof of claim 1.

11. The method of claim 10, wherein the serpin B13-related disorder is diabetes.

12. The method of claim 11, wherein the diabetes is type I diabetes, type 2 diabetes, or diabetes in patients with chronic pancreatitis who undergo total pancreatectomy with autologous islet transplantation and still remain insulin dependent.

13. The method of claim 11, wherein the diabetes is type I diabetes.

14. The method of claim 10, wherein the serpin B13-related disorder is inflammatory or central nervous system disease.

15. The method of claim 10, wherein the serpin B13-related disorder is a bone fracture, wound healing, hair loss, multiple sclerosis, or lupus.
